SEQUENCE LISTING (101 sequences)

SEQ ID NO: 1 - DNA, 20 bp, Artificial Sequence
  Example 1 desired product oligonucleotide sequence (“target”)
ggccaaacctcggcttacct

SEQ ID NO: 2 - DNA, 23 bp, Artificial Sequence
  Example 1-4, 6, 10 and 11 template oligonucleotide sequence
tttaggtaagccgaggtttggcc

SEQ ID NO: 3 - protein, 487 aa, Enterobacteria phage T4
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 4 - protein, 488 aa, Artificial Sequence
  Wild type T4 DNA ligase protein sequence (when fused to CBD)
GSILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 5 - RNA, 20 nt, Artificial Sequence
  Example 2 target sequence
ggccaaaccucggcuuaccu

SEQ ID NO: 6 - protein, 482 aa, Unknown
  Description of Unknown Enterobacteria phase CC31
MILDIINEIASIGSTKEKEAIIRRHKDNELLKRVFRMTYDGKLQYYIKKWDTRPKGDIHLTLEDMLYLLEEKLAKRVVTGNAAKEKLEIALSQTSDADAEVVKKVLLRDLRCGASRSIANKVWKNLIPEQPQMLASSYDEKGIEKNIKFPAFAQLKADGARAFAEVRGDELDDVKILSRAGNEYLGLDLLKQQLIEMTKEARERHPGGVMIDGELVYHASTLPAGPLDDIFGDLPELSKAKEFKEESRTMSNGLANKSLKGTISAKEAAGMKFQVWDYVPLDVVYSEGKQSGFAYDVRFRALELMVQGYSQMILIENHIVHNLDEAKVIYRKYVDEGLEGIILKNIGAFWENTRSKNLYKFKEVITIDLRIVDIYEHSKQPGKAGGFYLESECGLIKVKAGSGLKDKPGKDAHELDRTRIWENKNDYIGGVLESECNGWLAAEGRTDYVKLFLPIAIKMRRDKDVANTFADIWGDFHEVTGL

SEQ ID NO: 7 - protein, 483 aa, Artificial Sequence
  Wild-type Enterobacteria phage CC31 DNA ligase protein sequence (when fused to CBD)
GSILDIINEIASIGSTKEKEAIIRRHKDNELLKRVFRMTYDGKLQYYIKKWDTRPKGDIHLTLEDMLYLLEEKLAKRVVTGNAAKEKLEIALSQTSDADAEVVKKVLLRDLRCGASRSIANKVWKNLIPEQPQMLASSYDEKGIEKNIKFPAFAQLKADGARAFAEVRGDELDDVKILSRAGNEYLGLDLLKQQLIEMTKEARERHPGGVMIDGELVYHASTLPAGPLDDIFGDLPELSKAKEFKEESRTMSNGLANKSLKGTISAKEAAGMKFQVWDYVPLDVVYSEGKQSGFAYDVRFRALELMVQGYSQMILIENHIVHNLDEAKVIYRKYVDEGLEGIILKNIGAFWENTRSKNLYKFKEVITIDLRIVDIYEHSKQPGKAGGFYLESECGLIKVKAGSGLKDKPGKDAHELDRTRIWENKNDYIGGVLESECNGWLAAEGRTDYVKLFLPIAIKMRRDKDVANTFADIWGDFHEVTGL

SEQ ID NO: 8 - protein, 497 aa, Unknown
  Description of Unknown Shigella phage Shf125875
MILDILNQIAAIGSTKTKQEILKKNKDNKLLERVYRLTYARGIQYYIKKWPGPGERSQAYGLLELDDMLDFIEFTLATRKLTGNAAIKELMGYIADGKPDDVEVLRRVMMRDLEVGASVSIANKVWPGLIQLQPQMLASAYDEKLITKNIKWPAFAQLKADGARCFAEVRDDGVQFFSRAGNEYHGLTLLADELMEMTKEARERHPNGVLIDGELVYHSFDIKKAVSSGNDLSFLFGDNEESEEVQVADRSTSNGLANKSLQGTISPKEAEGMVLQAWDYVPLDEVYSDGKIKGQKYDVRFAALENMAEGFKRIEPIENQLVHNLDEAKVVYKKYVDQGLEGIILKNRDSYWENKRSKNLIKFKEVIDIALEVVGYYEHSKDPNKLGGVELVSRCRRITTDCGSGFKDTTHKTVDGVKVLIPLDERHDLDRERLMAEAREGKLIGRIADCECNGWVHSKGREGTVGIFLPIIKGFRFDKTEADSFEDVFGPWSQTGL

SEQ ID NO: 9 - protein, 498 aa, Artificial Sequence
  Wild-type Shigella phage Shf125875 DNA ligase protein sequence (when fused to CBD)
GSILDILNQIAAIGSTKTKQEILKKNKDNKLLERVYRLTYARGIQYYIKKWPGPGERSQAYGLLELDDMLDFIEFTLATRKLTGNAAIKELMGYIADGKPDDVEVLRRVMMRDLEVGASVSIANKVWPGLIQLQPQMLASAYDEKLITKNIKWPAFAQLKADGARCFAEVRDDGVQFFSRAGNEYHGLTLLADELMEMTKEARERHPNGVLIDGELVYHSFDIKKAVSSGNDLSFLFGDNEESEEVQVADRSTSNGLANKSLQGTISPKEAEGMVLQAWDYVPLDEVYSDGKIKGQKYDVRFAALENMAEGFKRIEPIENQLVHNLDEAKVVYKKYVDQGLEGIILKNRDSYWENKRSKNLIKFKEVIDIALEVVGYYEHSKDPNKLGGVELVSRCRRITTDCGSGFKDTTHKTVDGVKVLIPLDERHDLDRERLMAEAREGKLIGRIADCECNGWVHSKGREGTVGIFLPIIKGFRFDKTEADSFEDVFGPWSQTGL

SEQ ID NO: 10 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKRVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 11 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKGVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 12 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKKVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 13 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVILVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 14 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIKVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 15 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIQVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 16 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIVVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 17 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIRVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 18 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEAIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 19 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEKIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 20 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKRVIVVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 21 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKKVIEVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 22 - protein, 487 aa, Artificial Sequence
  Mutant ligase (T4 backbone) protein sequence
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKKVIHVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 23 - protein, 482 aa, Artificial Sequence
  Mutant ligase (Enterobacteria phage CC31 backbone - clone A4) protein sequence
MILDIINEIASIGSTKEKEAIIRRHKDNELLKRVFRMTYDGKLQYYIKKWDTRPKGDIHLTLEDMLYLLEEKLAKRVVTGNAAKEKLEIALSQTSDADAEVVKKVLLRDLRCGASRSIANKVWKNLIPEQPQMLASSYDEKGIEKNIKFPAFAQLKADGARAFAEVRGDELDDVKILSRAGNEYLGLDLLKQQLIEMTKEARERHPGGVMIDGELVYHASTLPAGPLDDIFGDLPELSKAKEFKEESRTMSNGLANKSLKGTISAKEAAGMKFQVWDYVPLDVVYSEGKQSGFAYDVRFRALELMVQGYSQMILIENHIVHNLDEAKVIYRKYVDEGLEGIILKNIGAFWENTRSKNLYKFKRVIVIDLRIVDIYEHSKQPGKAGGFYLESECGLIKVKAGSGLKDKPGKDAHELDRTRIWENKNDYIGGVLESECNGWLAAEGRTDYVKLFLPIAIKMRRDKDVANTFADIWGDFHEVTGL

SEQ ID NO: 24 - protein, 482 aa, Artificial Sequence
  Mutant ligase (Enterobacteria phage CC31 backbone) protein sequence
MILDIINEIASIGSTKEKEAIIRRHKDNELLKRVFRMTYDGKLQYYIKKWDTRPKGDIHLTLEDMLYLLEEKLAKRVVTGNAAKEKLEIALSQTSDADAEVVKKVLLRDLRCGASRSIANKVWKNLIPEQPQMLASSYDEKGIEKNIKFPAFAQLKADGARAFAEVRGDELDDVKILSRAGNEYLGLDLLKQQLIEMTKEARERHPGGVMIDGELVYHASTLPAGPLDDIFGDLPELSKAKEFKEESRTMSNGLANKSLKGTISAKEAAGMKFQVWDYVPLDVVYSEGKQSGFAYDVRFRALELMVQGYSQMILIENHIVHNLDEAKVIYRKYVDEGLEGIILKNIGAFWENTRSKNLYKFKKVIKIDLRIVDIYEHSKQPGKAGGFYLESECGLIKVKAGSGLKDKPGKDAHELDRTRIWENKNDYIGGVLESECNGWLAAEGRTDYVKLFLPIAIKMRRDKDVANTFADIWGDFHEVTGL

SEQ ID NO: 25 - protein, 482 aa, Artificial Sequence
  Mutant ligase (Enterobacteria phage CC31 backbone) protein sequence
MILDIINEIASIGSTKEKEAIIRRHKDNELLKRVFRMTYDGKLQYYIKKWDTRPKGDIHLTLEDMLYLLEEKLAKRVVTGNAAKEKLEIALSQTSDADAEVVKKVLLRDLRCGASRSIANKVWKNLIPEQPQMLASSYDEKGIEKNIKFPAFAQLKADGARAFAEVRGDELDDVKILSRAGNEYLGLDLLKQQLIEMTKEARERHPGGVMIDGELVYHASTLPAGPLDDIFGDLPELSKAKEFKEESRTMSNGLANKSLKGTISAKEAAGMKFQVWDYVPLDVVYSEGKQSGFAYDVRFRALELMVQGYSQMILIENHIVHNLDEAKVIYRKYVDEGLEGIILKNIGAFWENTRSKNLYKFKGVIFIDLRIVDIYEHSKQPGKAGGFYLESECGLIKVKAGSGLKDKPGKDAHELDRTRIWENKNDYIGGVLESECNGWLAAEGRTDYVKLFLPIAIKMRRDKDVANTFADIWGDFHEVTGL

SEQ ID NO: 26 - protein, 482 aa, Artificial Sequence
  Mutant ligase (Enterobacteria phage CC31 backbone) protein sequence
MILDIINEIASIGSTKEKEAIIRRHKDNELLKRVFRMTYDGKLQYYIKKWDTRPKGDIHLTLEDMLYLLEEKLAKRVVTGNAAKEKLEIALSQTSDADAEVVKKVLLRDLRCGASRSIANKVWKNLIPEQPQMLASSYDEKGIEKNIKFPAFAQLKADGARAFAEVRGDELDDVKILSRAGNEYLGLDLLKQQLIEMTKEARERHPGGVMIDGELVYHASTLPAGPLDDIFGDLPELSKAKEFKEESRTMSNGLANKSLKGTISAKEAAGMKFQVWDYVPLDVVYSEGKQSGFAYDVRFRALELMVQGYSQMILIENHIVHNLDEAKVIYRKYVDEGLEGIILKNIGAFWENTRSKNLYKFKGVILIDLRIVDIYEHSKQPGKAGGFYLESECGLIKVKAGSGLKDKPGKDAHELDRTRIWENKNDYIGGVLESECNGWLAAEGRTDYVKLFLPIAIKMRRDKDVANTFADIWGDFHEVTGL

SEQ ID NO: 27 - protein, 482 aa, Artificial Sequence
  Mutant ligase (Enterobacteria phage CC31 backbone) protein sequence
MILDIINEIASIGSTKEKEAIIRRHKDNELLKRVFRMTYDGKLQYYIKKWDTRPKGDIHLTLEDMLYLLEEKLAKRVVTGNAAKEKLEIALSQTSDADAEVVKKVLLRDLRCGASRSIANKVWKNLIPEQPQMLASSYDEKGIEKNIKFPAFAQLKADGARAFAEVRGDELDDVKILSRAGNEYLGLDLLKQQLIEMTKEARERHPGGVMIDGELVYHASTLPAGPLDDIFGDLPELSKAKEFKEESRTMSNGLANKSLKGTISAKEAAGMKFQVWDYVPLDVVYSEGKQSGFAYDVRFRALELMVQGYSQMILIENHIVHNLDEAKVIYRKYVDEGLEGIILKNIGAFWENTRSKNLYKFKRVIFIDLRIVDIYEHSKQPGKAGGFYLESECGLIKVKAGSGLKDKPGKDAHELDRTRIWENKNDYIGGVLESECNGWLAAEGRTDYVKLFLPIAIKMRRDKDVANTFADIWGDFHEVTGL

SEQ ID NO: 28 - protein, 497 aa, Artificial Sequence
  Mutant ligase (Shigella phage Shf125875 backbone) protein sequence
MILDILNQIAAIGSTKTKQEILKKNKDNKLLERVYRLTYARGIQYYIKKWPGPGERSQAYGLLELDDMLDFIEFTLATRKLTGNAAIKELMGYIADGKPDDVEVLRRVMMRDLEVGASVSIANKVWPGLIQLQPQMLASAYDEKLITKNIKWPAFAQLKADGARCFAEVRDDGVQFFSRAGNEYHGLTLLADELMEMTKEARERHPNGVLIDGELVYHSFDIKKAVSSGNDLSFLFGDNEESEEVQVADRSTSNGLANKSLQGTISPKEAEGMVLQAWDYVPLDEVYSDGKIKGQKYDVRFAALENMAEGFKRIEPIENQLVHNLDEAKVVYKKYVDQGLEGIILKNRDSYWENKRSKNLIKFKRVIVIALEVVGYYEHSKDPNKLGGVELVSRCRRITTDCGSGFKDTTHKTVDGVKVLIPLDERHDLDRERLMAEAREGKLIGRIADCECNGWVHSKGREGTVGIFLPIIKGFRFDKTEADSFEDVFGPWSQTGL

SEQ ID NO: 29 - protein, 298 aa, Paramecium bursaria Chlorella virus 1
  Paramecium bursaria Chlorella virus PBCV-1
MAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDR

SEQ ID NO: 30 - DNA, 23 bp, Artificial Sequence
  Example 5, 8 and 9 template oligonucleotide sequence
tttggtgcgaagcagactgaggc

SEQ ID NO: 31 - DNA, 23 bp, Artificial Sequence
  Example 5 template oligonucleotide sequence
tttggtgcgaagcagagtgaggc

SEQ ID NO: 32 - DNA, 23 bp, Artificial Sequence
  Example 5 template oligonucleotide sequence
tttggtgcgaagcagattgaggc

SEQ ID NO: 33 - DNA, 23 bp, Artificial Sequence
  Example 5 template oligonucleotide sequence
tttggtgcgaagcagaatgaggc

SEQ ID NO: 34 - DNA, 23 bp, Artificial Sequence
  Example 5 template oligonucleotide sequence
tttggtgcgaagcagtctgaggc

SEQ ID NO: 35 - DNA, 23 bp, Artificial Sequence
  Example 5 template oligonucleotide sequence
tttggtgcgaagcagtgtgaggc

SEQ ID NO: 36 - DNA, 23 bp, Artificial Sequence
  Example 5 template oligonucleotide sequence
tttggtgcgaagcagtttgaggc

SEQ ID NO: 37 - DNA, 23 bp, Artificial Sequence
  Example 5 template oligonucleotide sequence
tttggtgcgaagcagtatgaggc

SEQ ID NO: 38 - DNA, 23 bp, Artificial Sequence
  Example 5 template oligonucleotide sequence
tttggtgcgaagcagcctgaggc

SEQ ID NO: 39 - DNA, 23 bp, Artificial Sequence
  Example 5 template oligonucleotide sequence
tttggtgcgaagcagcgtgaggc

SEQ ID NO: 40 - DNA, 23 bp, Artificial Sequence
  Example 5 template oligonucleotide sequence
tttggtgcgaagcagcttgaggc

SEQ ID NO: 41 - DNA, 23 bp, Artificial Sequence
  Example 5 template oligonucleotide sequence
tttggtgcgaagcagcatgaggc

SEQ ID NO: 42 - DNA, 23 bp, Artificial Sequence
  Example 5 template oligonucleotide sequence
tttggtgcgaagcaggctgaggc

SEQ ID NO: 43 - DNA, 23 bp, Artificial Sequence
  Example 5 template oligonucleotide sequence
tttggtgcgaagcagggtgaggc

SEQ ID NO: 44 - DNA, 23 bp, Artificial Sequence
  Example 5 template oligonucleotide sequence
tttggtgcgaagcaggttgaggc

SEQ ID NO: 45 - DNA, 23 bp, Artificial Sequence
  Example 5 template oligonucleotide sequence
tttggtgcgaagcaggatgaggc

SEQ ID NO: 46 - DNA, 20 bp, Artificial Sequence
  Example 14 “20 mer” oligonucleotide sequence
gccucagtctgcttcgcacc

SEQ ID NO: 47 - DNA, 36 bp, Artificial Sequence
  Example 7 template oligonucleotide sequence
tttggtgcgaagcagaaggtaagccgaggtttggcc

SEQ ID NO: 48 - protein, 298 aa, Paramecium bursaria Chlorella virus 1
  Paramecium bursaria Chlorella virus NE-JV-4
MAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYSDRVEDMKNYITAHPHILDHEQVKIIPLIPVEINNITELLQYERDVLSKGFEGVMIRKPDGKYKFGRSTLKEGILLKMKQFKDAEATIISMTALFKNTNTKTKDNFGYSKRSTHKNGKVEEDVMGSIEVDYDGVVFSIGTGFDADQRRDFWQNKESYIGKMVKFKYFEMGSKDCPRFPVFIGIRHEEDH

SEQ ID NO: 49 - protein, 298 aa, Paramecium bursaria Chlorella virus 1
  Paramecium bursaria Chlorella virus NYs1
MTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEEDC

SEQ ID NO: 50 - protein, 301 aa, Paramecium bursaria Chlorella virus 1
  Paramecium bursaria Chlorella virus NE-JV-1
MTAIQKPLLAASFKKLTVADVKYPVFATPKLDGIRALKIDGAFVSRTFKPIRNRAIADALQDLLPNGSDGEILSGSTFQDASSAVMTAKAGIGANTIFYWFDYVKDDPNKPYLDRMTDMENYLKERPEILNDDRIKIVPLIPKKIETKDELDTFEKICLDQGFEGVMIRSGAGKYKFGRSTEKEGILIKIKQFEDDEAVVIGFTPMQTNTNDKSMNELGDMKRSSHKDGKVNLDTLGALEVDWNGITFSIGTGFDHALRDKLWSERDKLIGKIVKFKYFAQGVKTAPRFPVFIGFRDPDDM

SEQ ID NO: 51 - protein, 300 aa, Acanthocystis turfacea Chlorella virus 1
  Acanthocystis turfacea Chlorella virus Canal-1
MAIQKPLLAASLKKMSVGDLTFPVFATPKLDGIRALKVGGTIVSRTFKPVRNSAISEVLASILPDGSDGEILSGKTFQESTSTVMTADAGLGSGTMFFWFDYVKDDPNKGYLDRIADMKSFTDRHPEILKDKRVTIVPLFPKKIDTTEELHEFEKWCLDQGFEGVMVRNAGGKYKFGRSTEKEQILVKIKQFEDDEAVVIGVSALQTNTNDKKLNQLGEMRRTSHQDGKVELEMLGALDVDWNGIRFSIGTGFDRDTRVDLWKRREGVIGKIVKFKYFSQGIKTAPRFPVFLGFRDKDDM

SEQ ID NO: 52 - protein, 300 aa, Acanthocystis turfacea Chlorella virus 1
  Acanthocystis turfacea Chlorella virus Br0604L
MAIQKPLLAASLKKLSVDDLTFPVYATPKLDGIRALKIDGTLVSRTFKPIRNTTISKVLTSLLPDGSDGEILSGKTFQDSTSTVMSADAGIGSGTTFFWFDYVKDDPNKGYLDRIADIKKFIDCRPEILKDSRVIIVPLFPKKIDTAEELNVFEKWCLDQGFEGVMVRNAGGKYKFGRSTEKEQILVKIKQFEDDEAVVIGVSALQTNTNDKKVNELGEMRRTSHQDGKVDLDMLGALDVDWNGIRFGIGTGFDKDTREDLWKRRDSIIGKIVKFKYFSQGVKTAPRFPVFLGFRDKNDM

SEQ ID NO: 53 - protein, 300 aa, Acanthocystis turfacea Chlorella virus 1
  Acanthocystis turfacea Chlorella virus NE-JV-2
MAIQKPLLAASLKKLSVDDLTFPVYATPKLDGIRALKIDGTIVSRTFKPIRNTTISNVLMSLLPDGSDGEILSGKTFQDSTSTVMSADAGIGSGTTFFWFDYVKDDPDKGYLDRIADMKKFVDSHPEILKDRRVTIVPLIPKKIDTVEELNVFEQWCLDQGFEGVMVRNAGGKYKFGRSTEKEQILVKIKQFEDDEAVVIGVSALQTNVNDKKMNELGDMRRTSHKDGKIDLEMLGALDVEWNGIRFGIGTGFDKDTREDLWKKRDSIIGKVVKFKYFSQGIKTAPRFPVFLGFRDENDM

SEQ ID NO: 54 - protein, 300 aa, Acanthocystis turfacea Chlorella virus 1
  Acanthocystis turfacea Chlorella virus TN603.4.2
MAIQKPLLAASLKKMSVDNLTFPVYATPKLDGIRALKIDGTLVSRTFKPIRNTTISKVLASLLPDGSDGEILSGKTFQDSTSTVMTTDAGIGSDTTFFWFDYVKDDPDKGYLDRIADMKTFVDQHPEILKDSCVTIVPLFPKKIDTPEELHVFEKWCLDQGFEGVMVRTAGGKYKFGRSTEKEQILVKIKQFEDDEAVVIGVSALQTNTNDKKLNQLGEMRRTSHQDGKVDLDMLGALDVDWNGIRFSIGTGFDKDTREDLWKQRDSIVGKVVKFKYFSQGIKTAPRFPVFLGFRDENDM

SEQ ID NO: 55 - protein, 300 aa, Acanthocystis turfacea Chlorella virus 1
  Acanthocystis turfacea Chlorella virus GM0701.1
MAIQKPLLAASLKKMSVDDLTFPVYTTPKLDGIRALKIDGTLVSRTFKPVRNSAISEVLASLLPDGSDGEILSGKTFQDSTSTVMTTDAGIGSDTTFFWFDYVKDDPNKGYLDRIADMKTFIDQHPEMLKDNHVTIVPLIPKKIDTVEELNIFEKWCLDQGFEGVMVRNAGGKYKFGRSTEKEQILVKIKQFEDDEAVVIGVSALQTNTNDKKLNQLGEMRRTSHQDGKIDLEMLGALDVDWNGIRFSIGTGFDRDTRVDLWKRRDGIVGRTIKFKYFGQGIKTAPRFPVFLGFRDKDDM

SEQ ID NO: 56 - protein, 287 aa, Unknown
  Description of Unknown Synechococcus phage S-CRM01
MLAGNFDPKKAKFPYCATPKIDGIRFLMVNGRALSRTFKPIRNEYIQKLLSKHLPDGIDGELTCGDTFQSSTSAIMRIAGEPDFKAWIFDYVDPDSTSILPFIERFDQISDIIYNGPIPFKHQVLGQSILYNIDDLNRYEEACLNEGYEGVMLRDPYGTYKFGRSSTNEGILLKVKRFEDAEATVIRIDEKMSNQNIAEKDNFGRTKRSSCLDGMVPMETTGALFVRNSDGLEFSIGSGLNDEMRDEIWKNKSSYIGKLVKYKYFPQGVKDLPRHPVFLGFRDPDDM

SEQ ID NO: 57 - protein, 322 aa, Unknown
  Description of Unknown marine sediment metagenome
MDAHELMKLNEYAERQNQKQKKQITKPMLAASLKDITQLDYSKGYLATQKLDGIRALMIDGKLVSRTFKPIRNNHIREMLEDVLPDGADGEIVCPGAFQATSSGVMSANGEPEFIYYMFDYVKDDITKEYWRRTQDMVQWLINQGPTRTPGLSKLKLLVPTLIKNYDHLKTYETECIDKGFEGVILRTPDSPYKCGRSTAKQEWLLKLKRFADDEAVVIGFTEKMHNDNEATKDKFGHTVRSSHKENKRPAGTLGSLIVRDIKTEIEFEIGTGFDDELRQKIWDARPEWDGLCVKYKHFAISGVKEKPRFPSFIGVRDVEDM

SEQ ID NO: 58 - protein, 691 aa, Mycobacterium tuberculosis
  Mycobacterium tuberculosis (strain ATCC 25618/H37Rv)
MSSPDADQTAPEVLRQWQALAEEVREHQFRYYVRDAPIISDAEFDELLRRLEALEEQHPELRTPDSPTQLVGGAGFATDFEPVDHLERMLSLDNAFTADELAAWAGRIHAEVGDAAHYLCELKIDGVALSLVYREGRLTRASTRGDGRTGEDVTLNARTIADVPERLTPGDDYPVPEVLEVRGEVFFRLDDFQALNASLVEEGKAPFANPRNSAAGSLRQKDPAVTARRRLRMICHGLGHVEGFRPATLHQAYLALRAWGLPVSEHTTLATDLAGVRERIDYWGEHRHEVDHEIDGVVVKVDEVALQRRLGSTSRAPRWAIAYKYPPEEAQTKLLDIRVNVGRTGRITPFAFMTPVKVAGSTVGQATLHNASEIKRKGVLIGDTVVIRKAGDVIPEVLGPVVELRDGSEREFIMPTTCPECGSPLAPEKEGDADIRCPNARGCPGQLRERVFHVASRNGLDIEVLGYEAGVALLQAKVIADEGELFALTERDLLRTDLFRTKAGELSANGKRLLVNLDKAKAAPLWRVLVALSIRHVGPTAARALATEFGSLDAIAAASTDQLAAVEGVGPTIAAAVTEWFAVDWHREIVDKWRAAGVRMVDERDESVPRTLAGLTIVVTGSLTGFSRDDAKEAIVARGGKAAGSVSKKTNYVVAGDSPGSKYDKAVELGVPILDEDGFRRLLADGPASRT

SEQ ID NO: 59 - protein, 676 aa, Enterococcus faecalis
  Enterococcus faecalis (strain ATCC 700802/V583)
MEQQPLTLTAATTRAQELRKQLNQYSHEYYVKDQPSVEDYVYDRLYKELVDIETEFPDLITPDSPTQRVGGKVLSGFEKAPHDIPMYSLNDGFSKEDIFAFDERVRKAIGKPVAYCCELKIDGLAISLRYENGVFVRGATRGDGTVGENITENLRTVRSVPMRLTEPISVEVRGECYMPKQSFVALNEEREENGQDIFANPRNAAAGSLRQLDTKIVAKRNLNTFLYTVADFGPMKAKTQFEALEELSAIGFRTNPERQLCQSIDEVWAYIEEYHEKRSTLPYEIDGIVIKVNEFALQDELGFTVKAPRWAIAYKFPPEEAETVVEDIEWTIGRTGVVTPTAVMAPVRVAGTTVSRASLHNADFIQMKDIRLNDHVIIYKAGDIIPEVAQVLVEKRAADSQPYEMPTHCPICHSELVHLDEEVALRCINPKCPAQIKEGLNHFVSRNAMNIDGLGPRVLAQMYDKGLVKDVADLYFLTEEQLMTLDKIKEKSANNIYTAIQGSKENSVERLIFGLGIRHVGAKAAKILAEHFGDLPTLSRATAEEIVALDSIGETIADSVVTYFENEEVHELMAELEKAQVNLTYKGLRTEQLAEVESPFKDKTVVLTGKLAQYTREEAKEKIENLGGKVTGSVSKKTDIVVAGEDAGSKLTKAESLGVTVWNEQEMVDALDASHF

SEQ ID NO: 60 - protein, 670 aa, Haemophilus influenzae
  Haemophilus influenzae (strain ATCC 51907/DSM 11121/ KW20/Rd)
MTNIQTQLDNLRKTLRQYEYEYHVLDNPSVPDSEYDRLFHQLKALELEHPEFLTSDSPTQRVGAKPLSGFSQIRHEIPMLSLDNAFSDAEFNAFVKRIEDRLILLPKPLTFCCEPKLDGLAVSILYVNGELTQAATRGDGTTGEDITANIRTIRNVPLQLLTDNPPARLEVRGEVFMPHAGFERLNKYALEHNEKTFANPRNAAAGSLRQLDPNITSKRPLVLNAYGIGIAEGVDLPTTHYARLQWLKSIGIPVNPEIRLCNGADEVLGFYRDIQNKRSSLGYDIDGTVLKINDIALQNELGFISKAPRWAIAYKFPAQEELTLLNDVEFQVGRTGAITPVAKLEPVFVAGVTVSNATLHNGDEIERLNIAIGDTVVIRRAGDVIPQIIGVLHERRPDNAKPIIFPTNCPVCDSQIIRIEGEAVARCTGGLFCAAQRKEALKHFVSRKAMDIDGVGGKLIEQLVDRELIHTPADLFKLDLTTLTRLERMGAKSAENALNSLENAKSTTLARFIFALGIREVGEATALNLANHFKTLDALKDANLEELQQVPDVGEVVANRIFIFWREAHNVAVVEDLIAQGVHWETVEVKEASENLFKDKTVVLTGTLTQMGRNEAKALLQQLGAKVSGSVSSKTDFVIAGDAAGSKLAKAQELNITVLTEEEFLAQITR

SEQ ID NO: 61 - protein, 667 aa, Staphylococcus aureus
MADLSSRVNELHDLLNQYSYEYYVEDNPSVPDSEYDKLLHELIKIEEEHPEYKTVDSPTVRVGGEAQASFNKVNHDTPMLSLGNAFNEDDLRKFDQRIREQIGNVEYMCELKIDGLAVSLKYVDGYFVQGLTRGDGTTGEDITENLKTIHAIPLKMKEPLNVEVRGEAYMPRRSFLRLNEEKEKNDEQLFANPRNAAAGSLRQLDSKLTAKRKLSVFIYSVNDFTDFNARSQSEALDELDKLGFTTNKNRARVNNIDGVLEYIEKWTSQRESLPYDIDGIVIKVNDLDQQDEMGFTQKSPRWAIAYKFPAEEVVTKLLDIELSIGRTGVVTPTAILEPVKVAGTTVSRASLHNEDLIHDRDIRIGDSVVVKKAGDIIPEVVRSIPERRPEDAVTYHMPTHCPSCGHELVRIEGEVALRCINPKCQAQLVEGLIHFVSRQAMNIDGLGTKIIQQLYQSELIKDVADIFYLTEEDLLPLDRMGQKKVDNLLAAIQQAKDNSLENLLFGLGIRHLGVKASQVLAEKYETIDRLLTVTEAELVEIHDIGDKVAQSVVTYLENEDIRALIQKLKDKHVNMIYKGIKTSDIEGHPEFSGKTIVLTGKLHQMTRNEASKWLASQGAKVTSSVTKNTDVVIAGEDAGSKLTKAQSLGIEIWTEQQFVDKQNELNS

SEQ ID NO: 62 - protein, 652 aa, Streptococcus pneumoniae
  Streptococcus pneumoniae (strain P1031)
MNKRMNELVALLNRYATEYYTSDNPSVSDSEYDRLYRELVELETAYPEQVLADSPTHRVGGKVLDGFEKYSHQYPLYSLQDAFSREELDAFDARVRKEVAHPTYICELKIDGLSISLTYEKGILVAGVTRGDGSIGENITENLKRVKDIPLTLPEELDITVRGECYMPRASFDQVNQARQENGEPEFANPRNAAAGTLRQLDTAVVAKRNLATFLYQEASPSTRDSQEKGLKYLEQLGFVVNPKRILAENIDEIWNFIQEVGQERENLPYDIDGVVIKVNDLASQEELGFTVKAPKWAVAYKFPAEEKEAQLLSVDWTVGRTGVVTPTANLTPVQLAGTTVSRATLHNVDYIAEKDIRKDDTVIVYKAGDIIPAVLRVVESKRVSEEKLDIPTNCPSCNSDLLHFEDEVALRCINPRCPAQIMEGLIHFASRDAMNITGLGPSIVEKLFAANLVKDVADIYRLQEEDFLLLEGVKEKSAAKLYQAIQASKENSAEKLLFGLGIRHVGSKASQLLLQYFHSIENLYQADSEEVASIESLGGVIAKSLQTYFATEGSEILLRELKETGVNLDYKGQTVVADAALSGLTVVLTGKLERLKRSEAKSKLESLGAKVTGSVSKKTDLVVVGADAGSKLQKAQELGIQVRDEAWLESL

SEQ ID NO: 63 - protein, 374 aa, Unknown
  Description of Unknown Wild-type Enterobacteria phage T4 RNA ligase 1 protein sequence - ′Rnl1′
MQELFNNLMELCKDSQRKFFYSDDVSASGRTYRIFSYNYASYSDWLLPDALECRGIMFEMDGEKPVRIASRPMEKFFNLNENPFTMNIDLNDVDYILTKEDGSLVSTYLDGDEILFKSKGSIKSEQALMANGILMNINHHRLRDRLKELAEDGFTANFEFVAPTNRIVLAYQEMKIILLNVRENETGEYISYDDIYKDATLRPYLVERYEIDSPKWIEEAKNAENIEGYVAVMKDGSHFKIKSDWYVSLHSTKSSLDNPEKLFKTIIDGASDDLKAMYADDEYSYRKIEAFETTYLKYLDRALFLVLDCHNKHCGKDRKTYAMEAQGVAKGAGMDHLFGIIMSLYQGYDSQEKVMCEIEQNFLKNYKKFIPEGY

SEQ ID NO: 64 - protein, 334 aa, Unknown
  Description of Unknown Wild-type Enterobacteria phage T4 RNA ligase 2 protein sequence - ′Rnl2′
MFKKYSSLENHYNSKFIEKLYSLGLTGGEWVAREKIHGTNFSLIIERDKVTCAKRTGPILPAEDFFGYEIILKNYADSIKAVQDIMETSAVVSYQVFGEFAGPGIQKNVDYCDKDFYVFDIIVTTESGDVTYVDDYMMESFCNTFKFKMAPLLGRGKFEELIKLPNDLDSVVQDYNFTVDHAGLVDANKCVWNAEAKGEVFTAEGYVLKPCYPSWLRNGNRVAIKCKNSKFSEKKKSDKPIKAKVELSEADNKLVGILACYVTLNRVNNVISKIGEIGPKDFGKVMGLTVQDILEETSREGITLTQADNPSLIKKELVKMVQDVLRPAWIELVS

SEQ ID NO: 65 - protein, 389 aa, Unknown
  Description of Unknown Wild-type Aeromonas virus Aeh1 ligase protein sequence - ′RNA3′
MQSIKELYQNLINLCVDDNTKFYFAETVTSLGTKVRIFDYHVAGYNDWIRPDAMASRGIMFEMNGEIPVRIMSRPMDKFFNYSEVIGWEKLETAGNMKMPDLNKIAYVIDKRDGSLISTYLDISGEIKNLLLKSKASIRSNQANDASVWLYQEDHKDLLEFCTAYAENGFTVNMEWTAPHNQIVLCYNEHQLRILNIRHNETGEYVDFGELQKDPTFVKYAADFFEVPGDGKAWIDEVYQMTGIEGFVVVMEDYQMFKLKTDWYVALHHTKDSINNSERLIYACAENCTDDLRQMFRDDENSLQKIEIFDNHFRDVVMDAMKKLTEAYEKYRGMERRDYAINMNNDFKNERHWFNIAMQMFAQRPDFSMTDEIVAVIKKYPKTFIPKGY

SEQ ID NO: 66 - protein, 383 aa, Unknown
  Description of Unknown Wild-type Klebsiella phage JD18 ligase protein sequence - ′RNA6′
MLELYKNLMNLCESSEVAKFFYKDFTGPMDGKFRVFSYHYASYSEWLKPDALECRGIMFEMDGDTPIRIASRPMEKFFNLNENPMTMGIDISDVEYIMDKADGSLVSSYVDDGYLYLKSKTSLYSDQARQASALLNSEEYSSLHQVILELALDGYTVNMEFVSPNNRVVLAYQEPQLFVLNVRNNTTGEYIKYDDLYANAKIRPYLINAYGISDPTTWVEGVRALEGVEGYIAVLNTGQRFKVKTEWYSALHHTKDSITSNERLFASVVSANSDDLRSLFAGDEYAIKKISAFEQAYLDYLGKSLELCQSFYDEYRGRARKDYAIAAQKATVNQRHLFGVIMNMYEGTVDVDKLLKDLERVFLKYWAGYVPKEYEKEIEISEE

SEQ ID NO: 67 - protein, 384 aa, Unknown
  Description of Unknown Wild-type Stenotrophomonas phage IME13 ligase protein sequence - ′RNA8′
MSRTIELFNNLMSVVEKSEKGNFYFKDVITSMGTKARIFSYFIASYTDWLQDDALECRGIMFELNDKNEPVRIMARPMQKFFNLKENPMTIGLDLTKMIGLMEKADGSLISSYHDQGYVYLKSKAAIFSDQANKAMALLNSPAYEKLRDAIVRAGSDFTFNMEYVGPSNRVVLPYEEEELIVLNVRHNETGQYVEFSTLLDDPLIRHRMIGVYPCPDWSKVTPEEWEAATRAETDIEGVIGIMPDGQLFKLKTDWYSSLHRTKDSINNNKALFQSIKERASDDLRGMFSDDNAALAKIEAFESAYIDTVAKYHKICAEVFLRFARFLIVEVFAIEAQARMKDCRYLFSIVMQQYGRDWDGELAVEKIEEHIIKEYATYVPMAYR

SEQ ID NO: 68 - protein, 394 aa, Unknown
  Description of Unknown Wild-type Escherichia phage JSE ligase protein sequence - ′RNA9′
MEHKLCQQKKTTKKVLALFKNLMALCDGSDTFYYKDEITAMQTRMRIFSYLYLSKPEMWNKPDALECRGIMFEIDDKDRPVRIASRPMEKFFNLGENDRARFKPEDVEMVMDKMDGSLVSTYIDNGYVQLKSKAALYSSQAERANGLLYSEKYADLRAKIADIGSDYTFNFEYTAPSNRIVVEYSEPSLTLLNVRHNVTGEYVPHQMLFADAILRPHLVNALQVDSARFSNILDEVRTAEGIEGIVARTKDGQMFKVKSEWYIGVHNIKNTSMFPNNLIYYVVESETDDLRAAYEGEPEALERIELFEEAFRSILRNAFTTATEFYNAHAGEDRKTYASNATIESRKHGDAQSYIFMCLMIAFDGLDYDRILASMKTYYLRNYKKLIPADKIDW

SEQ ID NO: 69 - protein, 385 aa, Unknown
  Description of Unknown Wild-type Acinetobacter phage Acj61 ligase protein sequence - ′RNA16′
MKELFDNLMALQDPNDISKFFYKDVVTQPGTKCRIFSYNYAAYSDWLLPGALESRGIMFELDADNQPVRVMARPMEKFFNLEENPFTMDLDLSQLEYAMTKADGSLISTYVDQGYLYTKSKGSISSSQAIESKQLLLDINYKPLAERALELAKDGFTCNFEYVAPNNRIVLNYAKKDLILLNVRHNETGEYVPMAELQKDPVLRNYLIDVYPPREDIDTNEMIKEIREMVDIEGFVFQHASGLKFKLKTEWYSNLHRVKDTLNNSEALFMVVVAGGSDDMKSLFTDDLSRTKIESFETAFLDYLKKTSNFVFDLQRQLIGSDRKTYAIECQTILRNTDQLELFGVMMELYKGADQEQTIKNINVVFMKNYKKYVPAGFETLKNEY

SEQ ID NO: 70 - protein, 381 aa, Unknown
  Description of Unknown Wild-type Vibrio phage VH7D ligase protein sequence - ′RNA20′
MNVQELYKNLMSLADDAEGKFFFADHLSPLGEKFRVFSYHIASYSDWLLPGALEARGIMFQLDDNDEMIRIVSRPMEKFFNLNENPFTMELDLTTTVQLMDKADGSLISTYLSGENFALKSKTSIFSEQAVAANRYIKKPENRDLWEFCDDCTQAGLTVNMEWCAPNNRIVLEYPEAKLVILNIRDNETGDYVSFDDIPQSALMRVKQWLVDEYDPATAHEPDFVEKLRDTKGIEGMILRLANGQSVKIKTQWYVDLHSQKDSVNVPKKLVTTILNGNHDDLYALFADDKPTIERIREFDSHVTKTLTNSFNAVRQFYARNRHLARKDYAIAGQKVLKPWEFGVAMIAYQKQTVEGVYESLVTAYLKRPELAIPEKYLNGV

SEQ ID NO: 71 - protein, 382 aa, Unknown
  Description of Unknown Wild-type Escherichia phage JS98 ligase protein sequence - ′RNA22′
MIELYDNLMTLVKNSTKSKFFFKDFQSALGVNYRIFSYNYASYSDWLEDGALECRGIMFEMDENGPVRIAARPMQKFFNLDENPLTIGLDLSQENIDLVMAKEDGSLISTFMDRQYLSVKSKGSIHSSMVHDSLRFLRLPENEAFAARLEEITKAGYTCNLEYVSPTNRIVLAYQETNLILLNVRNNETGEYIPYAELFKDGALRKHLVKSYELSEGDFVDNIRKQEGIEGFIFVLKDGTFFKLKTAWYSALHHTKDSINNNERLFEVVVAGGTDDLRGLFSTDSFAIEKINAFERIHLDYLEQSLALLEAAYSQLKGRDRKDYAVTGQLILKDFPGLFSILMQAYVDGINYDTVMDQINSVFLKNHKAQIPEKYLKEIVVE

SEQ ID NO: 72 - protein, 386 aa, Unknown
  Description of Unknown Wild-type Escherichia phage vB_EcoM_112 ligase protein sequence - ′RNA23′
MVLYSKHKRGYTMQELFNNLMELCKDSQRKFFYSDDVSASGRTYRIFSYNYASYSDWLLPDALECRGIMFEMDGEKPVRIASRPMEKFFNLNENPFTMNIDLNDVDYILTKEDGSLVSTYLDGDEILFKSKGSIKSEQALMANGILMNINHHQLRDRLKELAEDGFTANFEFVAPTNRIVLAYQEMKIILLNVRENETGEYISYDDIYKDAILRPYLVERYEIDSPKWVEEAKNAENIEGYVAVMKDGSHFKIKSDWYVSLHSTKSSLDNPEKLFKTIIDGASDDLKAMYADDEYSYRKIEAFETTYLKYLDRALFLVLDCHNKHCGKDRKTYAMEAQGVAKGAGMDHLFGIIMSLYQGYDSQEKVMCEIEQNFLKNYKKFIPEGY

SEQ ID NO: 73 - protein, 392 aa, Unknown
  Description of Unknown Wild-type Aeromonas phage CC2 ligase protein sequence - ′RNA28′
MKELYNNLLKLTEEHGDCFFFRDHWSSIGNHFRVFSYHIAGLTQWMLPDALECRGIMFELINGEPYRIASRPMEKFFNLAERQAWNLTNSGVVGLDKLELDYSNIDRYEDKADGSLMSSYHFIDPEDDNRINYMLKSKTSINSDQANDANRWLVNHTDLLDFIIDCEEAGYTVNLEWCSPKNQIVIMYPEESLKILNVRHRDTGEYYSNNSLIRSPVFRKYAVDQFMFEEGTDVNTAISNMYNETGIEGYILVMRDGSRVKIKTTSYVARHKLKDSITNNKDLVIAVAQGVSDDLRQLFLDDSLSLTKIQEFEDHVVSVAGSTYTKIREAHKACAGMERREYAISMQNTFKQDRMFFNIVMKMFQAPDLEVMPEIMSVIIKYPDEFVPTKWK

SEQ ID NO: 74 - protein, 374 aa, Unknown
  Description of Unknown Wild-type Enterobacteria phage RB69 ligase protein sequence - ′RNA29′
MEKLYYNLLSLCKSSSDRKFFYSDDVSPIGKKYRIFSYNFASYSDWLLPDALECRGIMFEMDGETPLRIASRPMEKFFNLNENPFTLSIDLNDVKYLMTKEDGSLVSTYLDGNMVRFKSKGSIKSDQAASATSILLDINHKDLADRLLELCNDGFTANFEYVAPSNKIVLTYPEKRLILLNIRDNNTGKYIEYDDIYLDPVFRKYLVDRFEAPEGDWVPGVKSSTNIEGYVAVMKDGSHFKLKTDWYVALHTTRDSISSPEKLFLAIMNGASDDLKAMYADDEFSFKKVELFEKAYLDFLDRSFYICLDAYDKHKGKDRKTYAIEAQAICKGAQSPWLFGIIMNLYQGGSKEQMMTALESVFIKNHKNFIPEGY

SEQ ID NO: 75 - protein, 388 aa, Unknown
  Description of Unknown Wild-type Acinetobacter phage Ac42 ligase protein sequence - ′RNA31
MNELYNNLMTLIEPGKMSRFFLRDAVTPFGTRVRMFGYNYASYTDWLLPDALEARGIMFEMDENDQPIRVMARPMEKFFNLGENPFTIDLDLSTIEYFMDKSDGSLISSYVDNDTLFMKSKMSIGSVQAVAARQVIQDYVHRDLHDRVLELAKDGFTCNFEYVAPDNRVVILYPERALVLLNVRNNETGEYVHIEELKRDPVLRRYLVNNYVIDPENFDQDTFVNDIYQMVDIEGYVFRLMTGQHVKIKTEWYKMLHYAKDTINNNEALFAITVAAQLDDVRSLYSDDYALGKINKFEEVFLGFLDTRLPILLNLHKELNGSSRKDYAIKSQTYFKQANELYLFGIFMQMFEGVPPREQLVEKLSEAFMKNYKLFVPPEYDKVVEYDN

SEQ ID NO: 76 - protein, 382 aa, Unknown
  Description of Unknown Wild-type Pectobacterium phage CBB ligase protein sequence - ′RNA33′
MRTKQIFDDLMNLTAKNDAFMWKDFVSPAGGLFRIFSYRLASYSDFLEPNALECRGSMFKVDDEGNFVGIASRTPMKFFNAYENPFTMYDKDTLSSEIAVVMDKLDGSIISTFMDVDFVVRTKSHASLHSDHAYNSTAMLIADKELYNEVHYAESMGYTVNMEYTSPEYRIVLPYQEDNLTVLNLRHRETGELLIGERLKEFSKILYERSVFAKHGEIDATFPMKETLKESIDAVRGMADIEGYVLILKDGRMCKIKTDWYCALHFTKDSINVDSRLYDAIITGASDDLKQMFSTDLYAMKKIEKMEQLIFSCYNKLVHDVESFYEENKHLERKEYALKVQSTLPNELGMPGLAFSLYASKPVDYKGQMLKYMKDVLVNFEV

SEQ ID NO: 77 - protein, 385 aa, Unknown
  Description of Unknown Wild-type Aeromonas phage phiAS5 ligase protein sequence - ′RNA35
MQSIKELYQNLVNLCVDDNTKFFFSETVTSLGTKVRIFDYHVAGYNDWIRPDAMACRGIMFEMNGEIPVRIMSRPMDKFFNYSEVIGWEKLETHGNMKMPDLSKIKFVIDKRDGSLISTFMDVDNLLLKSKGSIRSNQANDASVWLYQDDQADLLEFCRAYAKEGFTVNMEWTAPHNQIVLCYTDHQLRILNIRHNETGEYVDFAELQKDSVFRKYAADFYEVPQDGAEWIKNVYGMTGIEGYVVVMEDYQMFKLKTDWYVALHHTKDSINDSKRLISACAENATDDLRQMFRDDPNSLAKIEVFDAKFRDVVSSAMQALTNAYSKYKALERREYAISMTNEFKNERHWFNIAMQMFSTRPDFSLADEIVAVIKKYPEKFVPQGY

SEQ ID NO: 78 - protein, 376 aa, Unknown
  Description of Unknown Wild-type Salmonella phage vB_SenMS16 ligase protein sequence - ′RNA36
MKELFDNLMNLCNDTDESRFFYRDDISPSGLKYRIFSYNYASYSDWLLPDALECRGIMFEMIDGVPVRIASRPMEKFFNLNETPFTMNLDLSNAVHMMKKEDGSLVSSYLDGNILRFKSKSSLKSEQAYLSSAMLTSITHEALLWRLLELARDGFTANFEYVSPENRIVLAYQKKDLILLNIRENDTGAYVPYNEIAKDPVLRQYLVESYEIPEGDFVSDIKAMEGIEGYVFVMDNGLRFKLKTDWYTALHHTKDSITKNDRLFEVIVNNASDDLKGLFSNDAYSLKKINKFEEVYLDYLRRSLSFISTSYQKLRGLDRKTYAGEAKRLADAERLPFLFTILMLMFNDSMDYDTTIKKVNELFMKNYKTFIPKEYE

SEQ ID NO: 79 - protein, 381 aa, Unknown
  Description of Unknown Wild-type Vibrio phage KVP40 ligase protein sequence - ′RNA39′
MTTQELYNHLMTLTEDAEGKFFFADHISPLGEKLRVFSYHIASYSDWLLPGALEARGIMFQLDEQDKMVRIVSRPMEKFFNLNENPFTMDLDLTTTVQLMDKADGSLISTYLTGENFALKSKTSIFSEQAVAANRYIKLPENRDLWEFCDDLTQAGCTVNMEWCAPNNRIVLEYPEAKLVILNIRDNETGDYVSFDDIPLPALMRVKKWLVDEYDPETAHVDDFVEKLRATKGIEGMILRLANGQSVKIKTQWYVDLHSQKDSVNVPKKLVTTILNNNHDDLYALFADDKPTIDRIREFDSHVSKTVSASFHAVSQFYVKNRHMSRKDYAIAGQKALKPWEFGVAMIAYQKKTVEGVYEALVGAYLKRPELLIPEKYLNEA

SEQ ID NO: 80 - protein, 361 aa, Unknown
  Description of Unknown Wild-type Campylobacter virus CP220 ligase protein sequence - ′RNA49′
MNYLELKNMAENLKDSNYRSTKLNNFKFYTYIFSDYKNFKENNTFFIRGLMIDSKSILNKDTLAPGISIPMPKFFNINENEDWLLPDSTNLEDFTIVTKYDGSLMIPYEYDGIKFRTKMSIDNDQTKLANKYIKNNPDILDLIKNNPDTQYFFELISPLNRIVVDYNKTELKLIAELDLRTLEFKIHETNEFNFKDLNIKTLKDLKDYINTISNYEGVILQHKVTKKVYKLKTQEYLDLHNTMTNLDLKVIYKMILEETIDDVLPKLSPEAVAYIDSVSNSVKVKLNEILDSIDSNYIKTKDLETPALYIKDLNIDPIAKDCLFKLCKNKLNLDDVLNQVKKSMLKYNKLRDIKVFLKWIH

SEQ ID NO: 81 - protein, 380 aa, Unknown
  Description of Unknown Wild-type Pseudomonas phage phiPMW ligase protein sequence - ′RNA59′
MKVIEFLKNAPSITDGLASLHLELGIKAKIYEDEGLIVLNYSQIDSPKTHPIVQECRGLIIDNDLTVVARPFDRFFNYGEALNVMPEIDWENASIFEKVDGSLIKIYFHKGRWEVATRGTAFAESECMGHGITFKELVFNALKVHDDDGFQYLMNNAYLFRDTTYLFELTCVENRVVRHYHGYNLHFLAARDNVSGNYSEECRDWLRSPDCILYGIVKHPKRYALGSADEALQAAKELKNLDEGFIVYQNAVPIAKIKSPAYVAVHHIRGEGLNPKRIMELVLSGEHDEYLSYFPEDRPIIQPYVDSLLDMLNIIAVTYPRLNQATTPKAFAAAIKHAGIDKQKASVYFMARRDNKDPVQVFHGMKTTFKMDMLRKWMMV

SEQ ID NO: 82 - protein, 317 aa, Unknown
  Description of Unknown Wild-type Pseudomonas phage vB_PaeM_C2-10_Ab1 ligase protein sequence - ′RNA61′
MPNCRIEIRRSRMEGNLNIAMYKDLIANKLVTVKHFNGMSIIKYARKVFYENLWNEHPLLLEARGHVFDQHSGDCIVRPFEKVFNLGENGAGSFLHPKFRVRLIEKVNGFMFSVTKHNGSLIFSTTGSLTSDYVALGQKYVSNNPDDYIAGFTYNFEICSPDDPHIVEEEEGAYLIGIRDIFTGGQLSEYILDSHALGVTDHSSVKILRPEHIECSWEHAKGLLSSCEKEGYMVQTGLGTVKCKSTHYLGKKFIMRMGSKKVNSMYQDPAGFKQTLDEEFYPLVDFLVNEVEEVRFTEMTDAQRRLLIETYFDMARI

SEQ ID NO: 83 - protein, 368 aa, Unknown
  Description of Unknown Wild-type Butyrivibrio proteoclasticus ligase protein sequence - ′RNA82′
MKSRILEFIKNNPDTWEEKLNEKFIRTNHNGDLVCFKYATEADFSDPLVCEARGIIIDVAQLVVVCWPFDKFFNVQEKYAADIDWNSARVLEKIDGSMIKLFWYKGAWRFATSSTCDAKDAAIPGYNELTYADIIARAENVNEIPFEELNKDYTYIFELVSPLSQIVVRYEMTELFFLTARNNLTGEELDTELLQFRRPRSFALKSMNECLDAALALNKGDEIEDEGFVVVDEKHNRVKIKSPAYVAMHRLSTNKVFTVKRMAEFFCNGEDLSKLAKDFPANAHIIKYYDWQFAEMKHKAEDMMLYSRRLYEEYDHDRKAVAMTIKDSPYAWAGFRAIGNDKDITDIMAVLAPANVEKLIVEYPEISN

SEQ ID NO: 84 - RNA, 10 nt, Artificial Sequence
  Example 15 and 16 primer oligonucleotide sequence
acuccucggu

SEQ ID NO: 85 - RNA, 11 nt, Artificial Sequence
  Example 15 and 16 product oligonucleotide sequence
acuccucggua

SEQ ID NO: 86 - DNA, 10 bp, Artificial Sequence
  Example 17 primer oligonucleotide sequence
actcatcgat

SEQ ID NO: 87 - DNA, 11 bp, Artificial Sequence
  Example 17 product oligonucleotide sequence
actcatcgata

SEQ ID NO: 88 - protein, 384 aa, Artificial Sequence
  Mutant ligase (Stenotrophomonas phage IME13 backbone) protein sequence
MSRTIELFNNLMSVVEKSEKGNFYFKDVITSMGTKARIFSHKIASYTDWLQDDALECRGIMFELNDKNEPVRIMARPMQKFFNLKENPMTIGLDLTKMIGLMEKADGSLISSYHDQGYVYLKSKTSIFSDQANKAMALLNSPAYEKLRDAIVRAGSDFTFNMEYVGPSNRVVLPYEEEELIVLNVRHNETGQYVEFSTLLDDPLIRHRMIGVYPCPDWSKVTPEEWEAATRAETDIEGVIGIMPDGQLFKLKTDWYSSLGRTKFSINNNKALHQSIKERASVALRGMFSDDNAALAKIEAFESAYIDTVAKYHKICAEVFLRFARFLIVEVFAIEAQARMKDCRYLFSIVMQQYGRDWDGELAVEKIEEHIIKEYATYVPMAYR

SEQ ID NO: 89 - protein, 384 aa, Artificial Sequence
  Mutant ligase (Stenotrophomonas phage IME13 backbone) protein sequence
MSRTIELFNNLMSVVEKSEKGNFYFKDVITSMGTKARIFSHQIASYTDWLQDDALECRGIMFELNDKNEPVRIMARPMQKFFNLKENPMTIGLDLTKMIGLMEKADGSLISSYHDQGYVYLKSKTSIFSDQANKAMALLNSPAYEKLRDAIVRAGSDFTFNMEYVGPSNRVVLPYEEEELIVLNVRHNETGQYVEFSTLLDDPLIRHRMIGVYPCPDWSKVTPEEWEAATRAETDIEGVIGIMPDGQLFKLKTDWYSSLGRTKFSINNNKALHQSIKERASGALRGMFSDDNAALAKIEAFESAYIDTVAKYHKICAEVFLRFARFLIVEVFAIEAQARMKDCRYLFSIVMQQYGRDWDGELAVEKIEEHIIKEYATYVPMAYR

SEQ ID NO: 90 - protein, 384 aa, Artificial Sequence
  Mutant ligase (Stenotrophomonas phage IME13 backbone) protein sequence
MSRTIELFNNLMSVVEKSEKGNFYFKDVITSMGTKARIFSYFIASYTDWLQDDALECRGIMFELNDKNEPVRIMARPMQKFFNLKENPMTIGLDLTKMIGLMEKADGSLISSYHDQGYVYLKSKTSIFSDQANKAMALLNSPAYEKLRDAIVRAGSDFTFNMEYVGPSNRVVLPYEEEELIVLNVRHNETGQYVEFSTLLDDPLIRHRMIGVYPCPDWSKVTPEEWEAATRAETDIEGVIGIMPDGQLFKLKTDWYSSLGRTKYSINNNKALNQSIKERASVALRGMFSDDNAALAKIEAFESAYIDTVAKYHKICAEVFLRFARFLIVEVFAIEAQARMKDCRYLFSIVMQQYGRDWDGELAVEKIEEHIIKEYATYVPMAYR

SEQ ID NO: 91 - protein, 384 aa, Artificial Sequence
  Mutant ligase (Stenotrophomonas phage IME13 backbone) protein sequence
MSRTIELFNNLMSVVEKSEKGNFYFKDVITSMGTKARIFSHNIASYTDWLQDDALECRGIMFELNDKNEPVRIMARPMQKFFNLKENPMTIGLDLTKMIGLMEKADGSLISSYHDQGYVYLKSKTTIFSDQANKAMALLNSPAYEKLRDAIVRAGSDFTFNMEYVGPSNRVVLPYEEEELIVLNVRHNETGQYVEFSTLLDDPLIRHRMIGVYPCPDWSKVTPEEWEAATRAETDIEGVIGIMPDGQLFKLKTDWYSSLGRTKYSINNNKALHQSIKERASVDLRGMFSDDNAALAKIEAFESAYIDTVAKYHKICAEVFLRFARFLIVEVFAIEAQARMKDCRYLFSIVMQQYGRDWDGELAVEKIEEHIIKEYATYVPMAYR

SEQ ID NO: 92 - protein, 384 aa, Artificial Sequence
  Mutant ligase (Stenotrophomonas phage IME13 backbone) protein sequence
MSRTIELFNNLMSVVEKSEKGNFYFKDVITSMGTKARIFSHKIASYTDWLQDDALECRGIMFELNDKNEPVRIMARPMQKFFNLKENPMTIGLDLTKMIGLMEKADGSLISSYHDQGYVYLKSKAAIFSDQANKAMALLNSPAYEKLRDAIVRAGSDFTFNMEYVGPSNRVVLPYEEEELIVLNVRHNETGQYVEFSTLLDDPLIRHRMIGVYPCPDWSKVTPEEWEAATRAETDIEGVIGIMPDGQLFKLKTDWYSSLHRTKDSINNNKALFQSIKERASDDLRGMFSDDNAALAKIEAFESAYIDTVAKYHKICAEVFLRFARFLIVEVFAIEAQARMKDCRYLFSIVMQQYGRDWDGELAVEKIEEHIIKEYATYVPMAYR

SEQ ID NO: 93 - DNA, 11 bp, Artificial Sequence
  Example 26 product oligonucleotide sequence
actcatcgatc

SEQ ID NO: 94 - DNA, 11 bp, Artificial Sequence
  Example 26 product oligonucleotide sequence
actcatcgatt

SEQ ID NO: 95 - protein, 477 aa, Unknown
  Description of Unknown Wild type phosphatase (Pandalus borealis) protein sequence - ′SAP′
MEEDKAYWNKDAQDALDKQLGIKLREKQAKNVIFFLGDGMSLSTVTAARIYKGGLTGKFEREKISWEEFDFAALSKTYNTDKQVTDSAASATAYLTGVKTNQGVIGLDANTVRTNCSYQLDESLFTYSIAHWFQEAGRSTGVVTSTRVTHATPAGTYAHVADRDWENDSDVVHDREDPEICDDIAEQLVFREPGKNFKVIMGGGRRGFFPEEALDIEDGIPGEREDGKHLITDWLDDKASQGATASYVWNRDDLLAVDIRNTDYLMGLFSYTHLDTVLTRDAEMDPTLPEMTKVAIEMLTKDENGFFLLVEGGRIDHMHHANQIRQSLAETLDMEEAVSMALSMTDPEETIILVTADHGHTLTITGYADRNTDILDFAGISDLDDRRYTILDYGSGPGYHITEDGKRYEPTEEDLKDINFRYASAAPKHSATHDGTDVGIWVNGPFAHLFTGVYEENYIPHALAYAACVGTGRTFCD

SEQ ID NO: 96 - DNA, 11 bp, Artificial Sequence
  Example 27 product oligonucleotide sequence
actcatcgata

SEQ ID NO: 97 - DNA, 12 bp, Artificial Sequence
  Example 27 product oligonucleotide sequence
actcatcgataa

SEQ ID NO: 98 - DNA, 12 bp, Artificial Sequence
  Example 27 product oligonucleotide sequence
actcatcgataa

SEQ ID NO: 99 - DNA, 12 bp, Artificial Sequence
  Example 28 product oligonucleotide sequence
  misc_feature (4)..(4): n = 2′ deoxyInosine
actnagaccacg

SEQ ID NO: 100 - protein, 221 aa, Unknown
  Description of Unknown Wild type Archaeoglobus fulgidus endonuclease V endonuclease
MLQMNLEELRRIQEEMSRSVVLEDLIPLEELEYVVGVDQAFISDEVVSCAVKLTFPELEVVDKAVRVEKVTFPYIPTFLMFREGEPAVNAVKGLVDDRAAIMVDGSGIAHPRRCGLATYIALKLRKPTVGITKKRLFGEMVEVEDGLWRLLDGSETIGYALKSCRRCKPIFISPGSYISPDSALELTRKCLKGYKLPEPIRIADKLTKEVKRELTPTSKLK

SEQ ID NO: 101 - DNA, 7 bp, Artificial Sequence
  Example 28 product oligonucleotide sequence
gaccacg

(19) United States
(12) Patent Application Publication
(10) Pub. No.: US 2024/0287566 A1
(43) Pub. Date: Aug. 29, 2024

(54) NOVEL PROCESSES FOR THE PRODUCTION OF OLIGONUCLEOTIDES

(71) Applicant: GLAXOSMITHKLINE INTELLECTUAL PROPERTY DEVELOPMENT LIMITED, Stevenage (GB)

(72) Inventors: Andreas CRAMERI, Collegeville (GB); David Graham TEW, Stevenage (GB)

(21) Appl. No.: 16/771,362

(22) PCT Filed: Dec. 17, 2018
(86) PCT No.: PCT/EP18/85179

§ 371 Date: Jun. 10, 2020

(30) Foreign Application Priority Data
Dec. 19, 2017 (GB) 1721307.5

(51) Int. Cl. C12P 19/30; C12N 9/00; C12N 9/12; C12N 9/22; C12N 15/10; C12N 15/113

(52) U.S. Cl. CPC C12P 19/30; C12N 9/1264; C12N 9/22; C12N 9/93; C12N 15/1031; C12N 15/113; C12N 2310/11; C12N 2310/314; C12N 2310/315; C12N 2310/321; C12N 2310/322; C12N 2310/3231; C12N 2310/332; C12N 2310/341; C12Y 605/01003

(57) ABSTRACT

The invention relates to novel processes using enzymes for the production of oligonucleotides, wherein said processes are suitable for use in the production of chemically modified oligonucleotides, such as those for use in therapy.

### SEQUENCE LISTING

The instant application contains a Sequence Listing which has been submitted electronically in ASCII format and is hereby incorporated by reference in its entirety. Said ASCII copy, created on Apr. 26, 2024, is named PB66490-US_SL.txt and is 267,723 bytes in size.

### FIELD OF THE INVENTION

The invention relates to novel processes using enzymes for the production of oligonucleotides, wherein said processes are suitable for use in the production of chemically modified oligonucleotides, such as those for use in therapy.

### BACKGROUND TO THE INVENTION

The chemical synthesis of oligonucleotides and modified oligonucleotides via phosphoramidite chemistry is well established and has been the method of choice for synthesizing these defined sequence biopolymers for several decades. The synthetic process is usually run as a solid phase synthesis, whereby single nucleotides are added sequentially with the addition of each nucleotide requiring a cycle of several chemical steps to add and deprotect the growing oligonucleotide (“oligo”) in preparation for the subsequent step. At the end of the sequential addition of nucleotides, the oligo is released from the solid phase support, further deprotection takes place, and then the crude oligonucleotide is further purified by column chromatography.

While this method may be considered routine and can be automated, there are several shortcomings to this methodology, especially if the goal is to prepare oligonucleotides at large scale as would be needed for oligonucleotide therapeutics. These shortcomings include, but are not limited to:
1) Practical limitations inherent in the use of chromatography making it unsuitable for purifying large quantities of oligonucleotide. The use of chromatography at large scale is expensive and is difficult to achieve due to the limitations on column size and performance.
2) The number of errors accumulates with the length of the oligonucleotide being synthesized. Accordingly, the linear sequential nature of the current process results in a geometric decrease in yield. For example, if the yield for each cycle of nucleotide addition is 99% then the yield of a 20 mer would be 83%.
3) Scale limitations with synthetic oligonucleotide synthesizers and downstream purification and isolation equipment: at present the maximum amount of product that can be produced in a single batch is in the order of 10 kg.

There is a need, therefore, to both reduce (or ideally eliminate) column chromatography and perform the synthesis in a way which is not purely sequential in order to increase yield.

DNA polymerase is often used to synthesize oligonucleotides for use in molecular biology and similar applications. However, DNA polymerase is unsuitable for synthesizing therapeutic oligonucleotides because of both the relatively short lengths of the oligonucleotides and the need to discriminate between nucleotides with different deoxyribose or ribose modifications. For example, therapeutic oligonucleotides are often in the range of 20 to 25 nucleotides. DNA polymerase needs at least 7 or 8 nucleotides, and optimally 18 to 22 nucleotides, as a primer in each direction so there is little to be gained in trying to synthesize a therapeutic oligo if the primers are similar in size to the desired product. Also, DNA polymerase requires all nucleotides to be present in the reaction and it relies on Watson-Crick base pairing to align incoming nucleotides. Thus, DNA polymerase is unable to discriminate between any ordering of deoxyribose or ribose modifications, such as those required by a gapmer, and the result would be a mix of deoxyribose or ribose modifications at a given position.

### SUMMARY OF THE INVENTION

The invention provides a process for producing a single-stranded oligonucleotide product having at least one modified nucleotide residue, comprising:
a) providing a pool of oligonucleotides (I) that comprises segments of the product sequence, wherein at least one segment of the product sequence contains at least one modified nucleotide residue, and wherein each segment has been produced by enzymatic synthesis or solid phase synthesis;
b) providing a template oligonucleotide (II) complimentary to the sequence of the single-stranded oligonucleotide product, said template having a property that allows it to be separated from the product and recycled for use in future reactions;
c) contacting (I) and (II) in conditions to allow annealing of the segment oligonucleotides to the template oligonucleotide;
d) joining the segment oligonucleotides by using a ligase to form the product;
e) changing the conditions to denature the annealed template and any impurity oligonucleotide strands, and separating the impurities;
f) changing the conditions to denature the annealed template and product oligonucleotide strands, and separating the single-stranded oligonucleotide product; and
g) recycling the template oligonucleotide for use in future reactions.

The invention also provides a process for producing a double-stranded oligonucleotide product, wherein two complimentary single-stranded oligonucleotides, produced by the aforementioned process for producing a single-stranded oligonucleotide product, are mixed under conditions to allow annealing.

### DESCRIPTION OF FIGURES

FIG. 1 Schematic of the enzymatic production of segment oligonucleotides: a) 3′-extension for segment synthesis comprising a two-step reaction: addition and deprotection. The exemplary addition step involves ATP dependent ligation of nucleotide-3′,5′-bis(thio)phosphate on to the 3′-OH of a single-stranded nucleic acid primer and then deprotection of the 3′-phosphate on the single-stranded oligonucleotide by a phosphatase; and b) the exemplary 3′-extension (addition and deprotection) to produce a segment sequence followed by chain cleavage using a site-specific nuclease (e.g. endonuclease V-cleaves one base after inosine, i.e. at second phosphodiester bond 3′ to inosine) to release the segment.

FIG. 2 Schematic of the process of the invention, including the steps of ligating the segment oligonucleotides to form the product and changing the conditions to remove impurities.

FIG. 3 Schematic of multiple template configurations.

FIG. 4 Basic schematic of the process of the invention being carried out in a flow system.

FIG. 5 Detailed schematic of the process of the invention being carried out in a flow system: a) ligation chemistry section, b) ligation purification section, and c) alternative ligation chemistry section, and d) alternative purification section. N.B. sections a) and b) (alternatively c) and d)) can be performed in a single step e.g. collection vessel in a)=output from ligation step in b).

FIG. 6 Examples of impurities which may be generated during the process of the invention.

FIG. 7 Chromatogram showing the results of a ligation reaction using commercial NEB T4 ligase (SEQ ID NO:3) and unmodified DNA (Example 1).

FIG. 8 Chromatogram showing the results of a ligation reaction using PERLOZA bound T4 ligase (SEQ ID NO:4) and unmodified DNA (Example 1).

FIG. 9 Chromatogram showing the results of a ligation reaction using PERLOZA bound T4 ligase expressed according to Example 2 and 2′-OMe modified oligonucleotide fragments.

FIG. 10 HPLC traces for Example 4: Upper trace (a)—No ligase control. Lower trace (b)—clone A4. Product and template co-elute in this HPLC method. Two intermediate ligation fragments (segments) can be seen in the Clone A4 trace at 10.3 and 11.2 minutes.

FIG. 11 Schematic of the “tri-template hub” used in Example 13, comprising a support material referred to as the “hub” and three template sequences.

FIG. 12 Schematic of the semi-continuous ligation rig used in Example 13.

FIG. 13 Schematic of the dead-end filtration rig used in Example 14.

FIG. 14 Schematic of the cross-flow filtration rig used in Example 14.

FIG. 15 Chromatograms showing the results from the dead-end filtration experiments using the 10 kDa MWCO NADIR membrane at 60° C. in Example 14. Figure a) is a chromatogram of the retentate solution, which remained in the filtration cell and contained mainly tri-template hub, after two diafiltration volumes; and chromatogram b) is of the permeate, solution enriched in the product, after two diafiltration volumes.

FIG. 16 Chromatograms showing the results from the cross-flow filtration experiments using the 5 kDa MWCO Snyder membrane at 50° C. and 3.0 bar pressure, in Example 14. Figure a) is a chromatogram of the retentate solution, which contained mainly tri-template hub and product, after 20 diafiltration volumes; and Figure b) is a chromatogram of the permeate, which contained mainly segment oligonucleotides, after 20 diafiltration volumes.

FIG. 17 Chromatograms showing the results from the cross-flow filtration experiments using the 5 kDa MWCO Snyder membrane at 80° C. and 3.1 bar pressure, in Example 14. FIG. 17 shows a) a chromatogram of the retentate solution, which contained tri-template hub only, after 20 diafiltration volumes; and Figure b) is a chromatogram of the permeate solution, which contained the product only, after 2 diafiltration volumes.

### DETAILED DESCRIPTION OF THE INVENTION

### Definitions

As used herein, the term “oligonucleotide”, or “oligo” for short, means a polymer of nucleotide residues. These may be deoxyribonucleotides (wherein the resulting oligonucleotide is DNA), ribonucleotides (wherein the resulting oligonucleotide is RNA), modified nucleotides, or a mixture thereof. An oligonucleotide may be entirely composed of nucleotide residues as found in nature or may contain at least one nucleotide, or at least one linkage between nucleotides, that has been modified. Oligonucleotides can be single-stranded or double-stranded. An oligonucleotide of the invention may be conjugated to another molecule, e.g. N-Acetylgalactosamine (GalNAc) or multiples thereof (GalNAc clusters).

As used herein, the term “therapeutic oligonucleotide” means an oligonucleotide that has a therapeutic application, e.g. in the prevention or treatment of a condition or disease in a human or animal. Such an oligonucleotide typically contains one or more modified nucleotide residues or linkages. Therapeutic oligonucleotides act via one of several different mechanisms, including, but not limited to, antisense, splice-switching or exon-skipping, immunostimulation and RNA interference (RNAi), e.g. via microRNA (miRNA) and small interfering RNA (siRNA). A therapeutic oligonucleotide may be an aptamer. Therapeutic oligonucleotides will usually, but not always, have a defined sequence.

As used herein, the term “template” means an oligonucleotide with a sequence that is 100% complementary to the sequence of the target (or product) oligonucleotide.

Unless otherwise specified, as used herein, the term “complementary” means 100% complementary.

As used herein, the term “product” means the desired oligonucleotide, having a specific sequence, also referred to herein as a “target oligonucleotide”.

As used herein, the term “pool” refers to a group of oligonucleotides that may vary in sequence, may be shorter or longer than the target sequence, and may not have the same sequence as the target sequence. The pool of oligonucleotides may be the product of oligonucleotide synthesis, e.g. solid phase chemical synthesis via phosphoramidite chemistry or enzymatic synthesis, used with or without purification. The pool of oligonucleotides may be composed of segments of the target sequence. Each segment itself may be present as a pool of that segment and may be the product of oligonucleotide synthesis, e.g. solid phase chemical synthesis via phosphoramidite chemistry or enzymatic synthesis.

As used herein, the term “annealing” means the hybridisation of complementary oligonucleotides in a sequence specific manner, e.g. the pairing of two single-stranded oligonucleotides, via the hydrogen bonds of Watson and Crick base-pairing, to form a double-stranded oligonucleotide. “Conditions to allow for annealing” will depend on the Tₘ of the hybridised complementary oligonucleotides and will be readily apparent to a person skilled in the art. For example, the temperature for annealing may be below the Tₘ of the hybridised oligonucleotides.

Alternatively, the temperature for annealing may be close to the Tₘ of the hybridised oligonucleotides, e.g. +/−1, 2 or 3° C. The temperature for annealing is, in general, not higher than 10° C. above the Tₘ of the hybridised oligonucleotides. Specific conditions to allow for annealing are as outlined in the examples section.

As used herein, the term “denaturing” in relation to a double-stranded oligonucleotide is used to mean that the complementary strands are no longer annealed, i.e. the Watson and Crick base-pairing has been disrupted and the strands have dissociated. Denaturing occurs as a result of changing the conditions, for example, by raising the temperature, changing the pH, or changing the salt concentration of the buffering solution. Conditions for denaturing are well known to those skilled in the art. Denaturing a double-stranded oligonucleotide (a “duplex”) as described herein results in a single-stranded product or impurity oligonucleotide and a single-stranded template oligonucleotide.

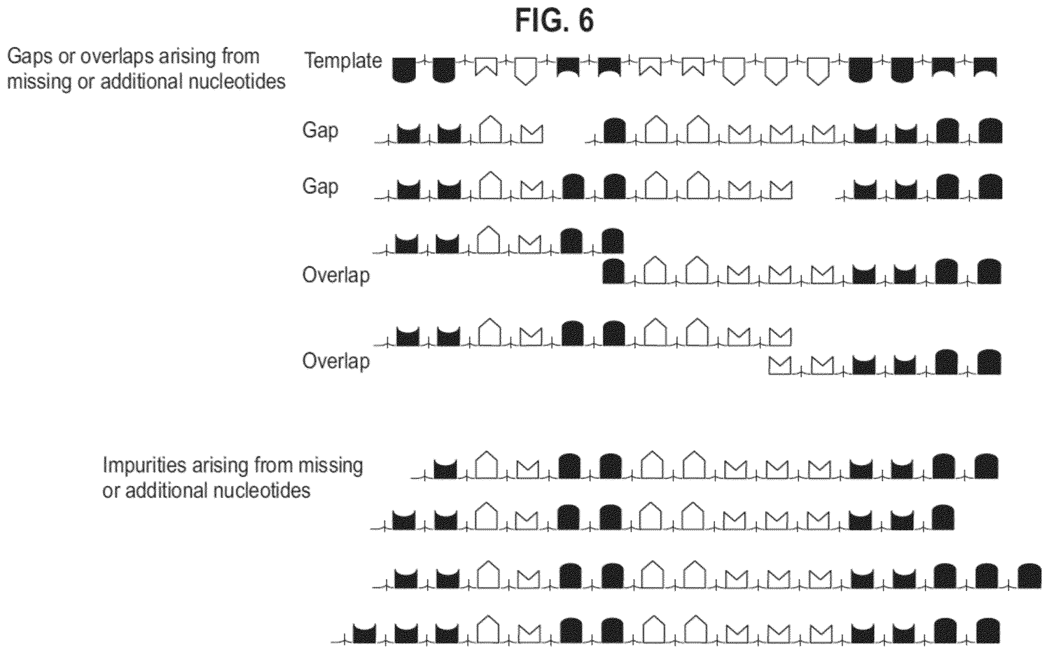

As used herein, the term “impurity” or “impurities” means oligonucleotides that do not have the desired product sequence. These oligonucleotides may include oligonucleotides that are shorter than the product (for example 1, 2, 3, 4, 5 or more nucleotide residues shorter), or that are longer than the product (for example 1, 2, 3, 4, 5 or more nucleotide residues longer). Where the production process includes a step whereby linkages are formed between segments, impurities include oligonucleotides that are remaining if one or more of the linkages fail to form. Impurities also include oligonucleotides where incorrect nucleotides have been incorporated, resulting in a mis-match when compared to the template. An impurity may have one or more of the characteristics described above. FIG. 6 shows some of the impurities that may occur.

As used herein, the term “segment” is a smaller portion of a longer oligonucleotide, in particular a smaller portion of a product or target oligonucleotide. For a given product, when all of its segments are annealed to its template and ligated together, the product is formed.

As used herein, the term “enzymatic ligation” means that the link between two adjacent nucleotides is formed enzymatically, i.e. by an enzyme. This linkage may be a naturally occurring phosphodiester bond (PO), or a modified linkage including, but not limited to, phosphorothioate (PS) or phosphoramidate (PA).

As used herein, the term “enzymatic synthesis” means the production of oligonucleotides, including segments and final product, using enzymes, e.g. ligases, transferases, phosphatases, and nucleases, in particular endonucleases. These enzymes may be wild-type enzymes or mutant enzymes. Within the scope of the present invention are mutant enzymes capable of acting on modified nucleotide or oligonucleotide substrates.

As used herein, the term “ligase” means an enzyme that catalyses the joining, i.e. covalent joining, of two oligonucleotide molecules, e.g. by formation of a phosphodiester bond between the 3′ end of one oligonucleotide (or segment) and the 5′ end of the same or another oligonucleotide (or segment). These enzymes are often referred to as DNA ligases or RNA ligases and utilise cofactors: ATP (eukaryotic, viral and archaeal DNA ligases) or NAD (prokaryotic DNA ligases). Despite their occurrence in all organisms, DNA ligases show a wide diversity of amino acid sequences, molecular sizes and properties (Nucleic Acids Research, 2000, Vol. 28, No. 21, 4051-4058). They are usually members of the Enzyme Class EC 6.5 as defined by the International Union of Biochemistry and Molecular Biology, i.e. ligases used to form phosphoric ester bonds. Within the scope of the invention is a ligase capable of joining an unmodified oligonucleotide to another unmodified oligonucleotide, a ligase capable of joining an unmodified oligonucleotide to a modified oligonucleotide (i.e. a modified 5′ oligonucleotide to an unmodified 3′ oligonucleotide, and/or an unmodified 5′ oligonucleotide to a modified 3′ oligonucleotide), as well as a ligase capable of joining a modified oligonucleotide to another modified oligonucleotide.

As used herein, the term “single-stranded ligase” or “ssLigase” means an enzyme, e.g. an RNA ligase, that is capable of catalysing the ATP-dependent ligation of (i) 5′-phosphorylated single-stranded RNA to the 3′-OH of a single-stranded acceptor RNA strand and (ii) the ligation of a single residue (including a modified residue), e.g. a nucleotide-3′,5′-bisphosphate, 3′,5′-thiobisphosphate or 3′-phosphate-5′ thiophosphate, to the 3′ end of RNA or a modified oligonucleotide (Modified Oligoribonucleotides: 17 (11), 2077-2081, 1978). An example of a ssLigase is T4 RNA ligase, which has also been shown to work on DNA substrates under certain conditions (Nucleic Acids research 7(2), 453-464, 1979). The natural function of T4 RNA ligase in Escherichia coli infected with T4 bacteriophage is to repair single-strand brakes to bacterial tRNA caused by bacterial defence mechanisms against viral attack. Within the scope of the invention is a ssLigase capable of joining an unmodified nucleotide to an unmodified oligonucleotide, a ssLigase capable of joining an unmodified nucleotide to a modified oligonucleotide, a ssLigase capable of joining a modified nucleotide to an unmodified oligonucleotide, as well as a ssLigase capable of joining a modified nucleotide to a modified oligonucleotide. A ssLigase according to the invention is a ligase that does not require a template oligonucleotide for ligation to occur, i.e. the ligation activity of the ligase is template-independent.

As used herein, a “thermostable ligase” is a ligase that is active at elevated temperatures, i.e. above human body temperature, i.e. above 37° C. A thermostable ligase may be active at, for example, 40° C.-65° C.; or 40° C.-90° C.; and so forth.

As used herein, a “transferase” means an enzyme that catalyses template independent joining of one nucleotide to another nucleotide or oligonucleotide. A transferase as described herein includes a terminal nucleotidyl transferase (TdT), also known as DNA nucleotidylexotransferase (DNTT) or terminal transferase. TdT is a specialised DNA polymerase that is expressed in immature, pre-B, pre-T-lymphoid cells where it enables V-D-J antibody gene junctional diversity. TdT catalyses the addition of nucleotides to the 3′ terminus of a DNA molecule. A transferase as described herein includes a non-naturally occurring or mutant TdT. Within the scope of the invention is a transferase capable of joining an unmodified nucleotide to an unmodified oligonucleotide, a transferase capable of joining an unmodified nucleotide to a modified oligonucleotide, a transferase capable of joining a modified nucleotide to an unmodified oligonucleotide, as well as a transferase capable of joining a modified nucleotide to a modified oligonucleotide.

As used herein, the term “phosphatase” means an enzyme that catalyses the hydrolysis of a phosphoester to produce an alcohol. Alkaline phosphatase non-specifically catalyses the dephosphorylation of 5′ and 3′ ends of DNA and RNA (and modified oligonucleotides) and also hydrolyses nucleotide triphosphates (NTPs and dNTPs) and is optimally active at alkaline pH environments.

As used herein, the term “sequence-specific endonuclease” or “site-specific endonuclease” are used interchangeably and mean a nuclease that specifically cleaves a single-stranded oligonucleotide at a particular position. For example, endonuclease V cleaves RNA one base after inosine, i.e. at the second phosphodiester bond 3′ to inosine. Other examples of site-specific endonucleases include the family of meganucleases, zinc finger nucleases, TALENs and Cas9.

As used herein, the term “primer” means an oligonucleotide sequence that is used as a starting point for synthesising a segment oligonucleotide of the invention. A primer comprises at least 3 nucleotides and may be attached to a support material.

As used herein, the term “modified nucleotide residue” or “modified oligonucleotide” means a nucleotide residue or oligonucleotide which contains at least one aspect of its chemistry that differs from a naturally occurring nucleotide residue or oligonucleotide. Such modifications can occur in any part of the nucleotide residue, e.g. sugar, base or phosphate. Examples of modifications of nucleotides are disclosed below.

As used herein, the term “modified ligase” means a ligase which differs from a naturally occurring, “wild-type”, ligase by one or more amino acid residues. Such ligases are not found in nature. Such ligases are useful in the novel processes of the invention. Examples of modified ligases are disclosed below. The terms “modified ligase” and “mutant ligase” are used interchangeably.

As used herein, the term “modified transferase” means a transferase which differs from a naturally occurring, “wild-type”, transferase by one or more amino acid residues. Such transferases are not found in nature. Such transferases are useful in the novel processes of the invention. The terms “modified transferase” and “mutant transferase” are used interchangeably.

As used herein, the term “gapmer” means an oligonucleotide having an internal “gap segment” flanked by two external “wing segments”, wherein the gap segment consists of a plurality of nucleotides that support RNase H cleavage and each wing segment consists of one or more nucleotides that are chemically distinct to the nucleotides within the gap segment.

As used herein, the term “support material” means a high molecular weight compound or material that increases the molecular weight of an oligonucleotide, e.g. the template or primer, thereby allowing it to be retained, e.g. when the impurities and products are separated from the reaction mixture.

As used herein “percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein. The query sequence may be 100% identical to the subject sequence, or it may include up to a certain integer number of nucleotide alterations as compared to the subject sequence such that the % identity is less than 100%. For example, the query sequence is at least 80, 85, 90, 95, 96, 97, 98, or 99% identical to the subject sequence.

### STATEMENT OF THE INVENTION

In an aspect of the invention a process for producing a single-stranded oligonucleotide product, having at least one modified nucleotide residue, is provided, said process comprising:
a) providing a pool of oligonucleotides (I) that comprises segments of the product sequence, wherein at least one segment of the product sequence contains at least one modified nucleotide residue, and wherein each segment has been produced by enzymatic synthesis or solid phase synthesis;
b) providing a template oligonucleotide (II) complimentary to the sequence of the single-stranded oligonucleotide product, said template having a property that allows it to be separated from the product and recycled for use in future reactions;
c) contacting (I) and (II) in conditions to allow annealing of the segment oligonucleotides to the template oligonucleotide;
d) joining the segment oligonucleotides by using a ligase to form the product;
e) changing the conditions to denature the annealed template and any impurity oligonucleotide strands, and separating the impurities;
f) changing the conditions to denature the annealed template and product oligonucleotide strands, and separating the single-stranded oligonucleotide product; and
g) recycling the template oligonucleotide for use in future reactions.

In an embodiment of the invention, one or more or all segment oligonucleotides is produced enzymatically.

In an embodiment of the invention, one or more or all segment oligonucleotides is produced using a single-stranded ligase (ssLigase). In an embodiment, the ssLigase is an RNA ligase. In an embodiment, the ssLigase is a T4 RNA ligase or modified T4 RNA ligase.

In another embodiment, the process for producing a segment oligonucleotide using a ssLigase comprises:
(i) adding a nucleotide, a dinucleotide, a trinucleotide or a tetranucleotide; which has either a phosphate, thiophosphate or dithiophosphate moiety at the 3′ end; and either a phosphate, thiophosphate or dithiophosphate moiety at the 5′ end; to the 3′-OH of a single-stranded oligonucleotide primer by using a ssLigase;
(ii) removing the 3′-phosphate moiety, 3′-thiophosphate moiety or 3′ dithiophosphate moiety by using a phosphatase;
(iii) repeating steps (i) and (ii) to extend the oligonucleotide to produce the segment sequence; and
(iv) releasing the segment oligonucleotide from the oligonucleotide primer using a sequence specific endonuclease.

In an embodiment of the invention, the process for producing a segment oligonucleotide, using a ssLigase, comprises adding a 3′,5′ nucleotide bisphosphate, having one or more of either of the phosphate oxygens substituted by sulphur, to the 3′-OH of a single-stranded oligonucleotide primer by using a ssLigase in step (i).

In an embodiment of the invention, the process for producing a segment oligonucleotide, using a ssLigase, comprises adding a 3′,5′ nucleotide bisphosphate, a 3′,5′ nucleotide thiophosphate (e.g. 3′,5′ bisthiophosphate or 3′-phosphate-5′-thiophosphate or 3′-thiophosphate-5′-phosphate) or a 3′,5′ nucleotide dithiophosphate (e.g. 3′,5′ bisdithiophosphate or 3′-phosphate-5′-dithiophosphate or 3′-dithiophosphate-5′-phosphate) to the 3′-OH of a single-stranded oligonucleotide primer by using a ssLigase in step (i).

In an embodiment of the invention, one or more segment oligonucleotides is produced using a transferase. In an embodiment of the invention, the process for producing a segment oligonucleotide using a transferase comprises:
(i) adding a nucleotide-5′-triphosphate, alpha-thiotriphosphate or alphadithiotriphosphate which has a protecting group on its 3′-OH to the 3′-OH of a single-stranded oligonucleotide primer by using a transferase;
(ii) deprotecting the 3′-position to regenerate the 3′-OH;
(iii) repeating steps (i) and (ii) to extend the oligonucleotide to produce the segment sequence;
(iv) releasing the segment oligonucleotide from the oligonucleotide primer using a sequence specific endonuclease.

In an embodiment of the invention, each segment is produced by enzymatic synthesis. In an embodiment of the invention, each segment is produced by using a ssLigase. In an embodiment of the invention, each segment is produced by using a transferase.

Using a ssLigase, e.g. an RNA ligase, a transferase or any other enzyme that is capable of adding a single nucleotide to a single-stranded oligonucleotide, in a template independent manner, allows synthesis of an oligonucleotide with a defined sequence. Such an approach could be used to produce the full oligonucleotide product by iteratively adding single bases. However, unless each synthetic cycle runs with 100% yield, sequence deletion errors will be incorporated into the final product. For example, if an oligonucleotide is extended by one nucleotide with 99% yield in a synthetic cycle, the remaining 1% will be available to react in subsequent synthetic cycles but the product formed will be one nucleotide shorter than the desired product. As the number of cycles increases then the error rate is compounded so, in this example, a 99% cycle yield would result in the formation of 20% of single base shortened sequences for the production of a 20mer.

According to the present invention, the sequential accumulation of errors is avoided by the following. Firstly, only short sequences, typically 5 to 8 nucleotides long, are synthesized by the addition of single nucleotides. This process results in short sequences that have a higher purity than long sequences as they are exposed to fewer cycles of error accumulation. Secondly, these short sequences are assembled on a complementary DNA template and then joined together. The use of a complementary template in conjunction with a ligase ensures that only short oligonucleotides that have both the correct length and the correct sequence are assembled. Accordingly, the processes of the invention results in both a higher overall yield and, importantly, higher overall sequence fidelity.

An embodiment of the invention provides a process as previously described herein, whereby denaturing the template and impurity duplex and/or denaturing the template and product duplex results from a temperature increase. In an embodiment, denaturation occurs as a result of changing the pH. In a further embodiment, denaturation occurs by changing the salt concentration in a buffering solution.

Yet another embodiment of the invention provides a process as previously disclosed herein, whereby the segment oligonucleotides are 3 to 15 nucleotides long. In a further embodiment of the invention the segments are 5 to 10 nucleotides long. In a further embodiment of the invention the segments are 5 to 8 nucleotides long. In a further embodiment of the invention the segments are 4, 5, 6, 7 or 8 nucleotides long. In a particular embodiment, there are three segment oligonucleotides: a 5′ segment that is 7 nucleotides long, a central segment that is 6 nucleotides long and a 3′ segment that is 7 nucleotides long, which when ligated together form an oligonucleotide that is 20 nucleotides long (a “20-mer”). In a particular embodiment, there are three segment oligonucleotides: a 5′ segment that is 6 nucleotides long, a central segment that is 8 nucleotides long and a 3′ segment that is 6 nucleotides long, which when ligated together form an oligonucleotide that is 20 nucleotides long (a “20-mer”). In a particular embodiment, there are three segment oligonucleotides: a 5′ segment that is 5 nucleotides long, a central segment that is 10 nucleotides long and a 3′ segment that is 5 nucleotides long, which when ligated together form an oligonucleotide that is 20 nucleotides long (a “20-mer”). In a particular embodiment, there are three segment oligonucleotides: a 5′ segment that is 4 nucleotides long, a central segment that is 12 nucleotides long and a 3′ segment that is 4 nucleotides long, which when ligated together form an oligonucleotide that is 20 nucleotides long (a “20-mer”). In a particular embodiment, there are four segment oligonucleotides: a 5′ segment that is 5 nucleotides long, a 5′-central segment that is 5 nucleotides long, a central-3′ segment that is 5 nucleotides long, and a 3′ segment that is 5 nucleotides long, which when ligated together form an oligonucleotide that is 20 nucleotides long (a “20-mer”).

One embodiment of the invention provides a process as previously described herein, whereby the product is 10 to 200 nucleotides long. In a further embodiment of the invention the product is 15 to 30 nucleotides long. In an embodiment of the invention, the product is 21, 22, 23, 24, 25, 26, 27, 28, 29 or 30 nucleotides long. In an embodiment of the invention, the product is 20 nucleotides long, a “20-mer”. In an embodiment of the invention, the product is 21 nucleotides long, a “21-mer”. In an embodiment of the invention, the product is 22 nucleotides long, a “22-mer”. In an embodiment of the invention, the product is 23 nucleotides long, a “23-mer”. In an embodiment of the invention, the product is 24 nucleotides long, a “24-mer”. In an embodiment of the invention, the product is 25 nucleotides long, a “25-mer”. In an embodiment of the invention, the product is 26 nucleotides long, a “26-mer”. In an embodiment of the invention, the product is 27 nucleotides long, a “27-mer”. In an embodiment of the invention, the product is 28 nucleotides long, a “28-mer”. In an embodiment of the invention, the product is 29 nucleotides long, a “29-mer”. In an embodiment of the invention, the product is 30 nucleotides long, a “30-mer”.

In an embodiment of the invention, the process is a process for producing a therapeutic oligonucleotide. In an embodiment of the invention, the process is a process for producing a single-stranded therapeutic oligonucleotide. In an embodiment of the invention, the process is a process for producing a double-stranded therapeutic oligonucleotide.

Another embodiment of the invention provides a process as previously disclosed herein, wherein the property that allows the template to be separated from the product is that the template is attached to a support material. In a further embodiment of the invention, the support material is a soluble support material. In a yet further embodiment of the invention the soluble support material is selected from the group consisting of: polyethylene glycol, a soluble organic polymer, DNA, a protein, a dendrimer, a polysaccharide, an oligosaccharide, and a carbohydrate. In an embodiment of the invention the support material is polyethylene glycol (PEG). In a further embodiment of the invention, the support material is an insoluble support material. In a further embodiment of the invention the support material is a solid support material. In a yet further embodiment, the solid support material is selected from the group consisting of: a glass bead, a polymeric bead, a fibrous support, a membrane, a streptavidin coated bead and cellulose. In an embodiment, the solid support material is a streptavidin coated bead. In a further embodiment, the solid support material is part of the reaction vessel itself, e.g. a reaction wall.

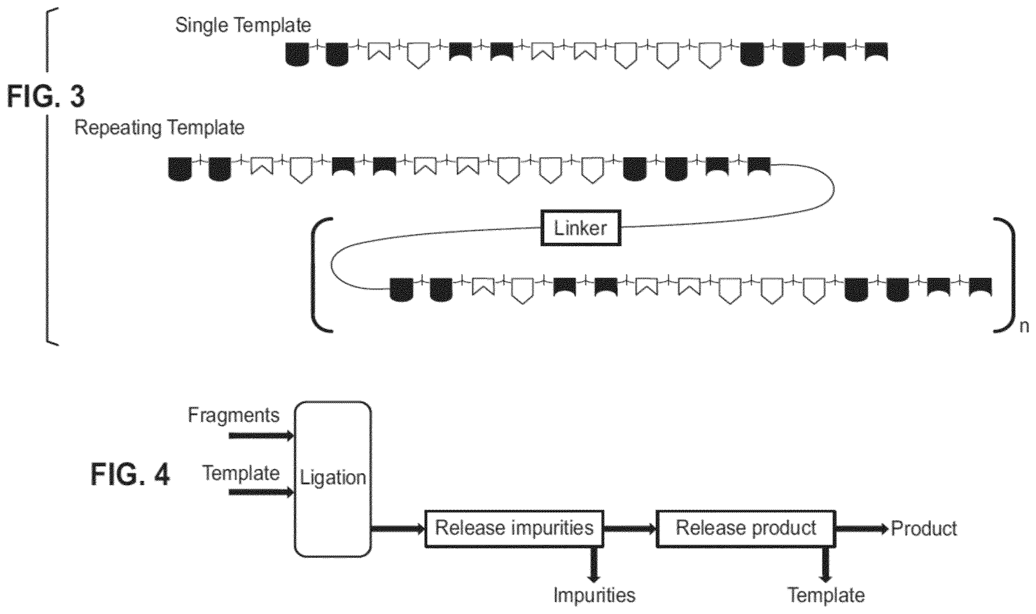

One embodiment of the invention provides a process as previously disclosed herein, wherein multiple, repeated copies of the template are attached in a continuous manner via a single attachment point to the support material. The multiple repeated copies of the template may be separated by a linker, e.g. as shown in FIG. 3. The multiple repeated copies of the template may be direct repeats, i.e. they are not separated by a linker.

In another embodiment of the invention, the template is attached to the support material at multiple attachment points.

Yet another embodiment of the invention provides a process as previously disclosed herein, wherein the property that allows the template to be separated from the product is the molecular weight of the template. For example, repeated copies of the template sequence may be present on a single oligonucleotide, with or without a linker sequence.

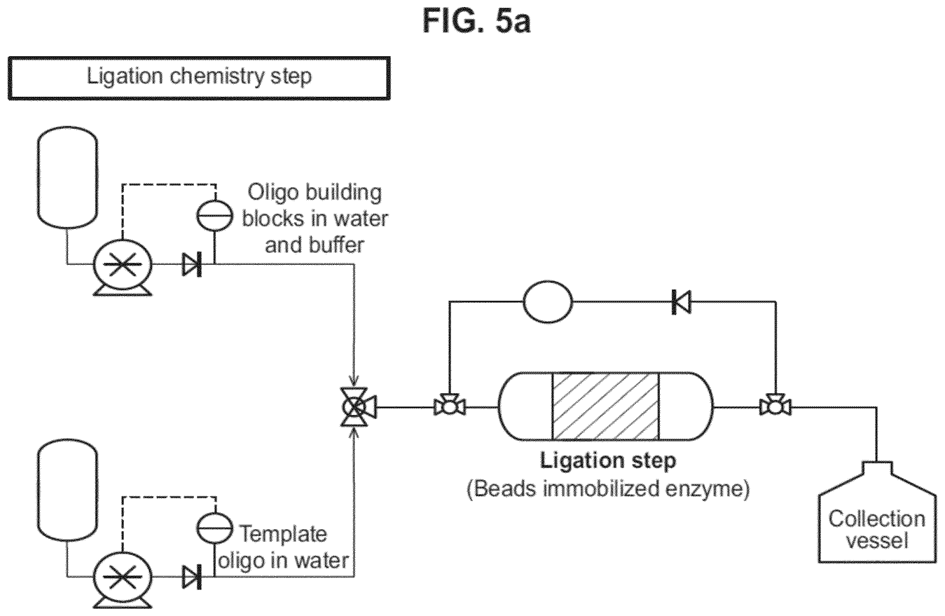

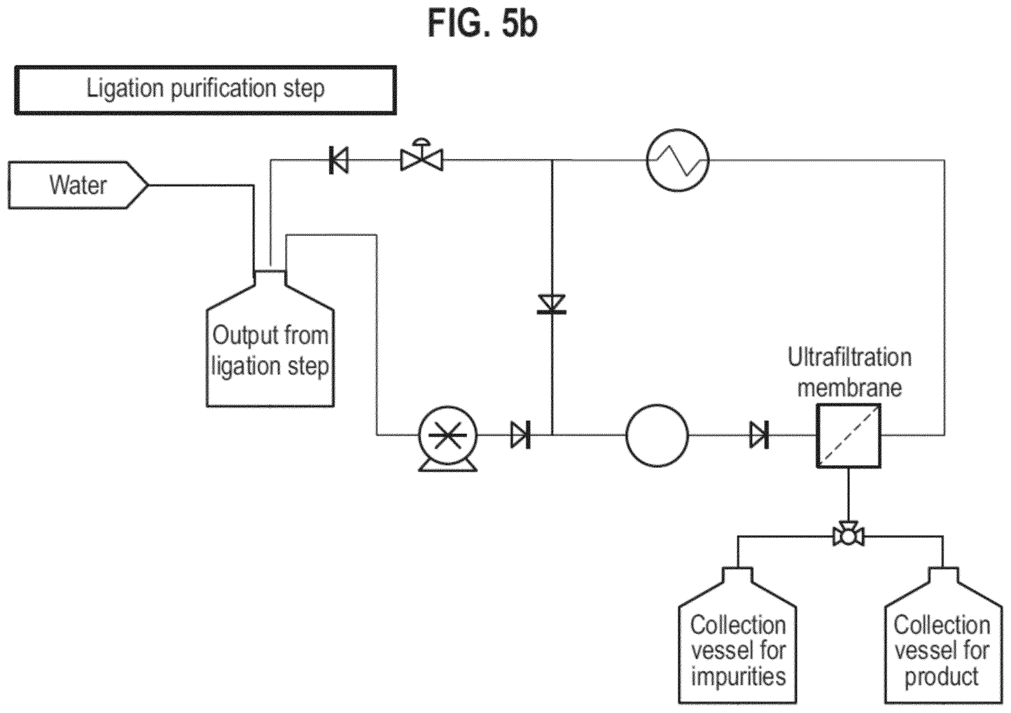

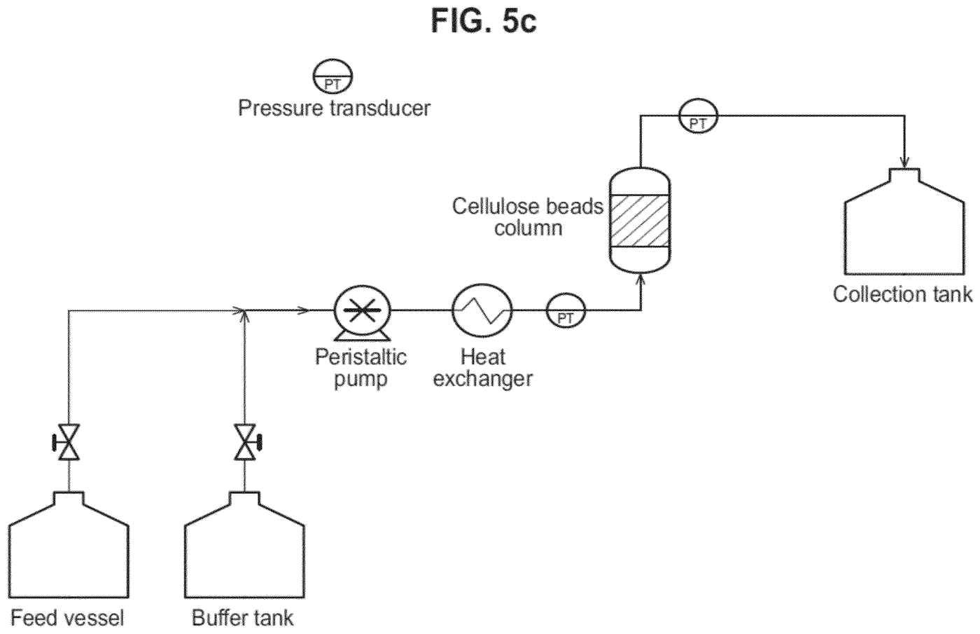

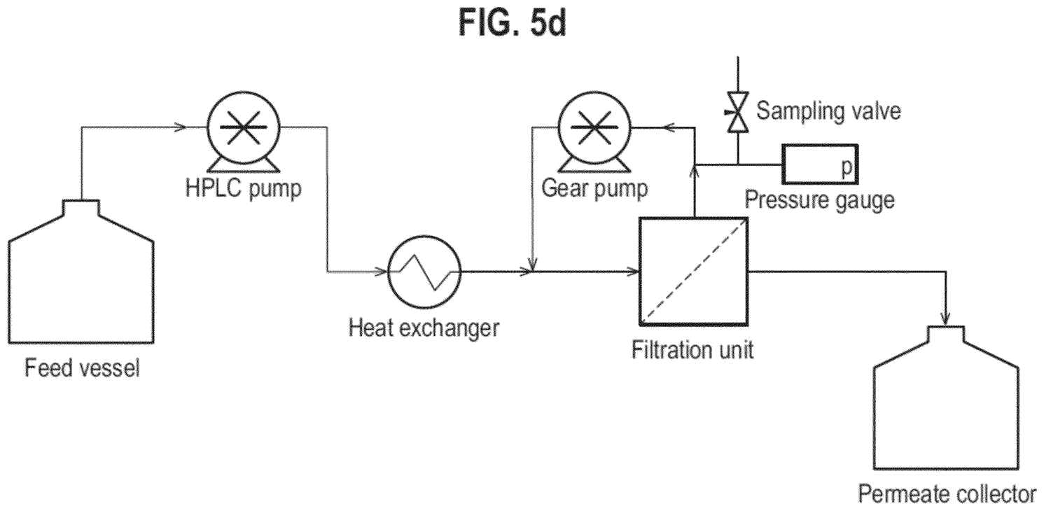

Another embodiment of the invention provides a process as previously disclosed herein, wherein the template, or the template and support material, are recycled for use in future reactions, for example as detailed below. Another embodiment of the invention provides a process as previously disclosed herein, wherein the reaction is carried out using a continuous or semi-continuous flow process, for example as shown in FIG. 4 or FIG. 5.

In an embodiment of the invention, the process is for large scale manufacture of oligonucleotides, in particular therapeutic oligonucleotides. In the context of the present invention, large scale manufacture of oligonucleotides means manufacture at a scale greater than or equal to 1 litre, e.g. the process is carried out in a 1 L or larger reactor. Alternatively, or in addition, in the context of the present invention large scale manufacture of oligonucleotides means manufacture at gram scale of product, in particular the production of greater than or equal to 10 grams of product. In an embodiment of the invention, the amount of oligonucleotide product produced is at gram scale. In an embodiment of the invention the amount of product produced is greater than or equal to: 1, 2, 3, 4, 5, 6, 7, 8, 9, 10, 20, 30, 40, 50, 60, 70, 80, 90 or 100 grams. In an embodiment of the invention, the amount of oligonucleotide product produced is greater than or equal to: 150, 200, 250, 300, 350, 400, 450, 500, 550, 600, 650, 700, 750, 800, 850, 900, 950 grams. In an embodiment of the invention, the amount of oligonucleotide product produced is 500 grams or greater. In an embodiment of the invention, the oligonucleotide product produced is at kilogram scale. In an embodiment of the invention, the amount of oligonucleotide product produced is 1 kg or more. In an embodiment of the invention, the amount of oligonucleotide product produced is greater than or equal to: 1, 2, 3, 4, 5, 6, 7, 8, 9, 10 kg. In an embodiment of the invention, the amount of oligonucleotide product produced is greater than or equal to: 15, 20, 25, 30, 35, 40, 45, 50, 55, 60, 65, 70, 75, 80, 85, 90, 95 or 100 kg.

In an embodiment of the invention, the amount of product produced is between 10 grams and 100 kg. In an embodiment of the invention, the amount of product produced is between 10 grams and 50 kg. In an embodiment of the invention, the amount of product produced is between 100 grams and 100 kg. In an embodiment of the invention, the amount of product produced is between 100 grams and 50 kg. In an embodiment of the invention, the amount of product produced is between 500 grams and 100 kg. In an embodiment of the invention, the amount of product produced is between 500 grams and 50 kg. In an embodiment of the invention, the amount of product produced is between 1 kg and 50 kg. In an embodiment of the invention, the amount of product produced is between 10 kg and 50 kg.

In an embodiment of the invention, oligonucleotide manufacture takes place at a scale greater than or equal to: 2, 3, 4, 5, 6, 7, 8, 9, 10 litres, e.g. in a 2, 3, 4, 5, 6, 7, 8, 9 or 10 L reactor. In an embodiment of the invention, oligonucleotide manufacture takes place at a scale greater than or equal to: 20, 25, 30, 35, 40, 45, 50, 55, 60, 65 70, 75, 80, 85, 90, 95, 100 litres, e.g. in a 20, 25, 30, 35, 40, 45, 50, 55, 60, 65 70, 75, 80, 85, 90, 95, 100 L reactor. In an embodiment of the invention, oligonucleotide manufacture takes place at a scale greater than or equal to: 150, 200, 250, 300, 350, 400, 450, 500, 550, 600, 650, 700, 750, 800, 850, 900, 950, 1000 litres, e.g. in 150, 200, 250, 300, 350, 400, 450, 500, 550, 600, 650, 700, 750, 800, 850, 900, 950, 1000 L reactor.

In an embodiment of the invention, the reactor volume is about 10,000 L, about 5000 L, about 2000 L, about 1000 L, about 500 L, about 125 L, about 50 L, about 20 L, about 10 L, or about 5 L.

In an embodiment of the invention, the reactor volume is between 5 and 10,000 L, between 10 and 5000 L, between 20 and 2000 L, or between 50 and 1000 L.

An oligonucleotide in accordance with the present invention may have at least one backbone modification, and/or at least one sugar modification and/or at least one base modification compared to an RNA or DNA-based oligonucleotide.

In an embodiment of the invention, one or more segment oligonucleotides have at least one backbone modification, and/or at least one sugar modification and/or at least one base modification compared to an RNA or DNA-based oligonucleotide. In an embodiment, all of the segment oligonucleotides have at least one backbone modification. In an embodiment, at least one of the segments has a completely modified backbone. In an embodiment of the invention, all of the segments have a completely modified backbone. In an embodiment, the backbone of all of the segment oligonucleotides consists of phosphorothioate linkages. In an embodiment, two or more of the segments have at least one sugar modification and/or at least one base modification compared to an RNA or DNA-based oligonucleotide. In an embodiment, the “wing” segments comprise at least one sugar modification. In an embodiment, the “wing” segments consist entirely of modified sugars. In an embodiment, the “wing” segments consist entirely of 2′-MOE-modified sugars.

One embodiment of the invention provides a process as previously disclosed herein, wherein the product contains at least 1 modified nucleotide residue. In a further embodiment, the modification is at the 2′ position of the sugar moiety.

Oligonucleotides used in the process of the invention may include sugar modifications, i.e. a modified version of the ribosyl moiety, such as 2′-O-modified RNA such as 2′-O-alkyl or 2′-O-(substituted)alkyl e.g. 2′-O-methyl, 2′-O-(2-cyanoethyl), 2′-O-(2-methoxy)ethyl (2′-MOE), 2′-O-(2-thiomethyl)ethyl, 2 2′-O-butyryl, 2′-O-propargyl, 2′-O-allyl, 2′-O-(3-amino)propyl, 2′-O-(3-(dimethylamino)propyl), 2′-O-(2-amino)ethyl, 2′-O-(2-(dimethylamino)ethyl); 2′-deoxy (DNA); 2′-O-(haloalkoxy)methyl (Arai K. et al. Bioorg. Med. Chem. 2011, 21, 6285) e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl](MOCE), 2′-O-[2-(N-methylcarbamoyl)ethyl](MCE), 2′-O-[2-(N, N-dimethylcarbamoyl)ethyl](DCME); 2′-halo e.g. 2′-F, FANA (2′-F arabinosyl nucleic acid); carbasugar and azasugar modifications; 3′-O-alkyl e.g. 3′-O-methyl, 3′-O-butyryl, 3′-O-propargyl; and their derivatives.

In an embodiment of the invention, the sugar modification is selected from the group consisting of 2′-Fluoro (2′-F), 2′-O-methyl (2′-OMe), 2′-O-methoxyethyl (2′-MOE), and 2′-amino. In a yet further embodiment, the modification is 2′-MOE.

Other sugar modifications include “bridged” or “bicyclic” nucleic acid (BNA), e.g. locked nucleic acid (LNA), xylo-LNA, α-L-LNA, β-D-LNA, cEt (2′-O,4′-C constrained ethyl) LNA, cMOEt (2′-O,4′-C constrained methoxyethyl) LNA, ethylene-bridged nucleic acid (ENA), tricyclo DNA; unlocked nucleic acid (UNA); cyclohexenyl nucleic acid (CeNA), altriol nucleic acid (ANA), hexitol nucleic acid (HNA), fluorinated HNA (F-HNA), pyranosyl-RNA (p-RNA), 3′-deoxypyranosyl-DNA (p-DNA); morpholino (as e.g. in PMO, PPMO, PMOPlus, PMO-X); and their derivatives.

Oligonucleotides used in the process of the invention may include other modifications, such as peptide-base nucleic acid (PNA), boron modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), oligonucleotides with integrated bases and backbones (ONIBs), pyrrolidine-amide oligonucleotides (POMs); and their derivatives.

In an embodiment of the invention, the modified oligonucleotide comprises a phosphorodiamidate morpholino oligomer (PMO), a locked nucleic acid (LNA), a peptide nucleic acid (PNA), a bridged nucleic acid (BNA) such as (S)-cEt-BNA, or a SPIEGELMER.

In a further embodiment, the modification is in the nucleobase. Base modifications include modified versions of the natural purine and pyrimidine bases (e.g. adenine, uracil, guanine, cytosine, and thymine), such as inosine, hypoxanthine, orotic acid, agmatidine, lysidine, 2-thiopyrimidine (e.g. 2-thiouracil, 2-thiothymine), G-clamp and its derivatives, 5-substituted pyrimidine (e.g. 5-methylcytosine, 5-methyluracil, 5-halouracil, 5-propynyluracil, 5-propynylcytosine, 5-aminomethyluracil, 5-hydroxymethyluracil, 5-aminomethylcytosine, 5-hydroxymethylcytosine, Super T), 2,6-diaminopurine, 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in US6683173. cPent-G, cPent-AP and Pr-AP were shown to reduce immunostimulatory effects when incorporated in siRNA (Peacock H. et al. J. Am. Chem. Soc. (2011), 133, 9200).

In an embodiment of the invention, the nucleobase modification is selected from the group consisting of 5-methyl pyrimidines, 7-deazaguanosines and abasic nucleotides. In an embodiment, the modification is a 5-methyl cytosine.

Oligonucleotides used in the process of this invention may include a backbone modification, e.g. a modified version of the phosphodiester present in RNA, such as phosphorothioate (PS), phosphorodithioate (PS2), phosphonoacetate (PACE), phosphonoacetamide (PACA), thiophosphonoacetate, thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methylboranophosphonothioate, and their derivatives. Another modification includes phosphoramidite, phosphoramidate, N3′→P5′ phosphoramidate, phosphordiamidate, phosphorothiodiamidate, sulfamate, dimethylenesulfoxide, sulfonate, triazole, oxalyl, carbamate, methyleneimino (MMI), and thioacetamido nucleic acid (TANA); and their derivatives.

In a further embodiment, the modification is in the backbone and is selected from the group consisting of: phosphorothioate (PS), phosphoramidate (PA) and phosphorodiamidate. In an embodiment of the invention, the modified oligonucleotide is a phosphorodiamidate morpholino oligomer (PMO). A PMO has a backbone of methylenemorpholine rings with phosphorodiamidate linkages. In an embodiment of the invention the product has a phosphorothioate (PS) backbone.

In an embodiment of the invention, the oligonucleotide comprises a combination of two or more modifications as disclosed above. A person skilled in the art will appreciate that there are many synthetic derivatives of oligonucleotides.

In an embodiment of the invention, the product is a gapmer. In an embodiment of the invention, the wing segments comprise backbone and sugar modifications and the central or ‘gap’ segment comprises backbone modifications, but no sugar modifications. In an embodiment of the invention, the 5′ and 3′ wings of the gapmer comprise or consist of 2′-MOE modified nucleotides. In an embodiment of the invention the gap segment of the gapmer comprises or consists of nucleotides containing hydrogen at the 2′ position of the sugar moiety, i.e. is DNA-like. In an embodiment of the invention the 5′ and 3′ wings of the gapmer consist of 2′MOE modified nucleotides and the gap segment of the gapmer consists of nucleotides containing hydrogen at the 2′ position of the sugar moiety (i.e. deoxynucleotides). In an embodiment of the invention the 5′ and 3′ wings of the gapmer consist of 2′MOE modified nucleotides and the gap segment of the gapmer consists of nucleotides containing hydrogen at the 2′ position of the sugar moiety (i.e. deoxynucleotides) and the linkages between all of the nucleotides are phosphorothioate linkages.

One embodiment of the invention provides a process as previously described herein, wherein the resulting product is greater than 90% pure. In a further embodiment, the product is greater than 95% pure. In a further embodiment, the product is greater than 96% pure. In a further embodiment, the product is greater than 97% pure. In a further embodiment, the product is greater than 98% pure. In a further embodiment, the product is greater than 99% pure. Purity of an oligonucleotide may be determined using any suitable method, e.g. high-performance liquid chromatography (HPLC) or mass spectrometry (MS), in particular, liquid chromatography-MS (LC-MS), HPLC-MS or capillary electrophoresis mass spectrometry (CEMS).

In an embodiment of the invention the oligonucleotide produced is an antisense oligonucleotide. In an embodiment of the invention the oligonucleotide produced is an aptamer. In an embodiment of the invention the oligonucleotide produced is a miRNA. In an embodiment of the invention, the product is a therapeutic oligonucleotide.

Yet another embodiment of the invention provides a process for producing double-stranded oligonucleotides, wherein 2 complimentary single-stranded oligonucleotides are produced by the method of any preceding embodiment and then mixed under conditions to allow annealing, such conditions being readily apparent to a skilled person. In an embodiment, the product is a siRNA.

In an embodiment of the invention, there are substantially no nucleotides in the reaction vessel for ligating the segments. In an embodiment of the invention, there are no nucleotides in the reaction vessel for ligating the segments. In another embodiment of the invention, the reaction vessel for ligating the segments does not comprise a pool of nucleotides, i.e. the reaction is substantially free to completely free of nucleotides.

The invention herein disclosed utilises the properties of oligonucleotide binding to provide an improved process for their production. By providing a template oligonucleotide with 100% complementarity to the target sequence, and controlling the reaction conditions so that the product can be released and separated under specific conditions, a product with a high degree of purity can be obtained.

Denaturing the Product (or Impurity):Template Duplex and Separating the Product (or Impurity) from the Template

Releasing the product (or any impurities) from the template requires the Watson-Crick base pairing between the template oligonucleotide strand and the product (or impurity) to be broken (i.e. denaturing the duplex). The product (or impurity) can then be separated from the template, which can occur as two separate steps, or as one combined step.

Releasing and separating the product (or impurity) can occur as one step, if the process is carried out in a column reactor. Running in a buffer that alters the pH or salt concentration, or contains a chemical agent that disrupts the base pairing (such as formamide or urea) will cause denaturation of the oligonucleotide strands, and the product (or impurity) will be eluted in the buffer.

When the process is carried out in other reaction vessels, the release and separation of the product (or impurity) can occur as a two-step process. First, the Watson-Crick base pairs are disrupted to denature the strands, and then the product (or impurity) is separated from the template, e.g. removed from the reaction vessel. When releasing and separating the product is carried out as a two-step process, the breaking of the Watson-Crick base pairs can be achieved by altering the buffer conditions (pH, salt) or by introducing a chemical disrupting agent (formamide, urea). Alternatively, raising the temperature will also cause the dissociation of the two strands, i.e. denaturation. The product (or impurities) can then be separated (and also removed from the reaction vessel, if desired) via methods including molecular weight-based separation, charge based separation, hydrophobicity-based separation, specific sequence-based separation or a combination of these methods.

When the process is carried out in a continuous or semi-continuous flow reactor, the release and separation of the product (or impurity) can be in either one step or two steps. For example, releasing and separating the product (or impurity) in one step could be affected by increasing the temperature to cause dissociation of the two strands, and separating the released strands on the basis of molecular weight in the same part of the reactor that is used to elevate the temperature. Releasing and separating the product (or impurity) in two steps could be affected by increasing the temperature to cause dissociation of the two strands in one part of the reactor and separating the released strands on the basis of molecular weight in a different part of the reactor.

Specifically Releasing and Separating Impurities from the Template, but Retaining the Product on the Template

Impurities arise when an incorrect nucleotide is incorporated into the oligonucleotide strand during chain extension, or when the chain extension reaction terminates early. Impurities also arise when the reaction includes the step of ligating segment oligonucleotides and one or more of the ligation steps fail to happen. The kinds of impurities which can arise are illustrated in FIG. 6.

The properties of Watson-Crick base pairing can be exploited to specifically release any impurities bound to the template prior to the release of the product. Each double-stranded oligonucleotide will dissociate under specific conditions, and those conditions are different for sequences which do not have 100% complementarity when compared to sequences with 100% complementarity. Determining such conditions is within the remit of a skilled person.

A common way of denaturing oligonucleotides is by raising the temperature. The temperature at which half of the base pairs are dissociated, i.e. when 50% of the duplex is in the single-stranded state, is called the melting temperature, Tₘ. The most reliable and accurate means of determining the melting temperature is empirically. However, this is cumbersome and not usually necessary. Several formulas can be used to calculate Tₘ values (Nucleic Acids Research 1987, 15 (13): 5069-5083; PNAS 1986, 83 (11): 3746-3750; Biopolymers 1997, 44 (3): 217-239) and numerous melting temperature calculators can be found on-line, hosted by reagent suppliers and universities. It is known that for a given oligonucleotide sequence, a variant with all phosphorothioate linkages will melt at a lower temperature than a variant with all phosphodiester linkages. Increasing the number of phosphorothioate linkages in an oligonucleotide tends to lower the Tₘ of the oligonucleotide for its intended target.

To specifically separate the impurities from a reaction mixture, first the melting temperature of the product: template duplex is calculated. Then the reaction vessel is heated to a first temperature, e.g. a temperature below the melting temperature of the product: template duplex, for example 1, 2, 3, 4, 5, 6, 7, 8, 9 or 10 degrees Centigrade below the melting temperature. This heating step causes the denaturing of oligonucleotides which are not the product, i.e. are not 100% complimentary to the template, from the template. These denatured oligonucleotides can then be removed from the reaction vessel using one of the methods disclosed above, e.g. molecular weight-based separation, charge based separation, hydrophobicity-based separation, specific sequence-based separation or a combination of these methods. Then, the reaction vessel will be raised to a second, higher, temperature, e.g. above the calculated melting temperature, for example 1, 2, 3, 4, 5, 6, 7, 8, 9 or 10 degrees Centigrade above the melting temperature, to cause the denaturing of the product from the template. The product can then be separated (and removed from the reaction vessel) using one of the methods disclosed above, e.g. molecular weight-based separation, charge-based separation, hydrophobicity-based separation, specific sequence-based separation or a combination of these methods.

A similar process can be used when the disrupting agent is an agent which causes a change in pH or salt concentration or is a chemical disrupting agent. The disrupting agent is increased in concentration until just below the concentration at which the product would dissociate, to cause the denaturing of oligonucleotides which are not the product from the template. These impurities can then be removed from the reaction vessel using one of the methods disclosed above. The disrupting agent is then increased in concentration to above the concentration at which the product dissociates from the template. The product can then be removed from the reaction vessel using one of the methods disclosed above.

The product obtained from a process such as disclosed above has a high degree of purity without the need for further purification steps. For example, the product obtained is greater than 95% pure.

### Properties of the Template

The template requires a property which allows it to be retained in the reaction vessel when the product is removed, to prevent it from becoming an impurity in the product. In other words, the template has properties that allow it to be separated from the product. In one embodiment of the invention, this retention is achieved by coupling the template to a supporting material. This coupling results in a template-support complex which has a high molecular weight, and can therefore be retained in the reaction vessel when impurities and product are removed, for example by filtration. The template can be coupled to a solid support material such as polymeric beads, fibrous supports, membranes, streptavidin coated beads and cellulose. The template can also be coupled to a soluble support material such as polyethylene glycol, a soluble organic polymer, DNA, a protein, a dendrimer, a polysaccharide, an oligosaccharide and a carbohydrate.

Each support material can have multiple points where a template can be attached, and each attachment point can have multiple templates attached, e.g. in the manner shown in FIG. 3.

The template may have a high molecular weight itself, without being attached to a support material, for example, it may be a molecule with multiple copies of the template, e.g. separated by a linker, in the manner shown in FIG. 3.

The ability to retain the template in the reaction vessel also allows the template to be recycled for future reactions, either by being recovered or by use in a continuous or semi-continuous flow process.

Methods of Separating the Template from the Product (or Impurities)

The properties of the template, as disclosed above, allow separation of the template and product, or separation of the template bound product and impurities. Molecular weight-based separation, charge-based separation, hydrophobicity-based separation, specific sequence-based separation or a combination of these methods can be used.

In the case where the template is attached to a solid support, separation of the template from the product, or separation of impurities from the product bound to the template, is achieved by washing the solid support under appropriate conditions as would be readily apparent to a person skilled in the art. In cases where the template is coupled to a soluble support or is itself composed of repeating template sequences, separation of template from product or separation of template bound product from impurities can be achieved by means of a molecular weight-based separation, for example by using techniques such as ultra-filtration or nano-filtration where the filter material is chosen so that the larger molecule is retained by the filter and the smaller molecule passes through. In cases where a single separation step of impurity from template product complex, or separation of product from template, is not efficient enough, multiple sequential filtration steps can be employed to increase separation efficiency and so generate a product that meets the desired purity.

It is desirable to provide a process for separation of such oligonucleotides which is efficient and applicable on an industrial production scale. “Therapeutic oligonucleotides: The state of the art in purification technologies” Sanghvi et. al. Current Opinion in Drug Discovery (2004) Vol. 7 No. 8 reviews processes used for oligonucleotide purification.

WO 01/55160 A1 discloses purification of oligonucleotides by forming imine linkages with contaminants and then removing the imine-linked impurities with chromatography or other techniques. “Size Fractionation of DNA Fragments Ranging from 20 to 30000 Base Pairs by Liquid/Liquid chromatography” Muller et al. Eur. J. Biochem (1982) 128-238 discloses use of a solid column of microcrystalline cellulose on which has been deposited a PEG/dextran phase for separation of nucleotide sequences. “Separation and identification of oligonucleotides by hydrophilic interaction chromatography.” Easter et. al. The Analyst (2010); 135(10) discloses separation of oligonucleotides using a variant of HPLC employing a solid silica support phase. “Fractionation of oligonucleotides of yeast soluble ribonucleic acids by countercurrent distribution” Doctor et al. Biochemistry (1965) 4(1) 49-54 discloses use of a dry solid column packed with dry DEAE-cellulose. “Oligonucleotide composition of a yeast lysine transfer ribonucleic acid” Madison et al; Biochemistry, 1974, 13(3) discloses use of solid phase chromatography for separation of nucleotide sequences.

Liquid-liquid chromatography is a known separation method. “Countercurrent Chromatography The Support-Free Liquid Stationary Phase” Billardello, B.; Berthod, A; Wilson & Wilson's Comprehensive Analytical Chemistry 38; Berthod, A., Ed.; Elsevier Science B.V.: Amsterdam (2002) pp 177-200 provides a useful general description of liquid-liquid chromatography. Various liquid-liquid chromatography techniques are known. One such technique is liquid-liquid counter current chromatography (termed herein “CCC”). Another known technique is centrifugal partition chromatography (termed herein “CPC”).

The above disclosed methods and those methods set out in WO 2013/030263 may be used to separate a product oligonucleotide, e.g. from the template and/or an impurity.

Ligases for Use in the Processes of the Invention, i.e. Ligation Step (d)

In an embodiment of the invention, the ligase is an ATP dependent ligase. ATP dependent ligases range in size from 30 to >100 kDa. In an embodiment of the invention, the ligase is an NAD dependent ligase. NAD dependent enzymes are highly homologous and are monomeric proteins of 70-80 kDa. In an embodiment of the invention, the ligase is a thermostable ligase. A thermostable ligase may be derived from a thermophilic bacterium.

In an embodiment of the invention, the ligase is a template-dependent ligase.

In an embodiment of the invention, the ligase is a wild-type Enterobacteria phage CC31 DNA ligase (SEQ ID NO:6) or a wild-type Shigella phage Shf125875 DNA ligase (SEQ ID NO:8).

In an embodiment of the invention, the ligase is a modified ligase. For example, a modified ligase includes a modified T4 DNA ligase, a modified Enterobacteria phage CC31 ligase, a modified Shigella phage Shf125875 ligase and a modified Chlorella ligase.

In an embodiment, wild-type T4 DNA ligase is modified at amino acid position 368 or amino acid position 371 of SEQ ID NO:3.

In an embodiment, the mutant ligase comprises or consists of SEQ ID NO:3 wherein the amino acid at position 368 is R or K.

In an embodiment, the mutant ligase comprises or consists of SEQ ID NO:3 wherein the amino acid at position 371 is any one of the following amino acids: L, K, Q, V, P, R.

In an embodiment, the corresponding residue(s) disclosed above in relation to T4 DNA ligase are mutated in any one of Enterobacteria phage CC31 ligase, Shigella phage Shf125875 ligase and Chlorella ligase. Conserved regions of DNA ligases are disclosed in Chem. Rev. 2006, 106, 687-699 and Nucleic Acids Research, 2000, Vol. 28, No. 21, 4051-4058. In an embodiment, the ligase is modified in a linker region.

In an embodiment of the invention, the ligase comprises or consists of SEQ ID NO:23 or a ligase with at least 90% sequence identity thereto, excluding a wild-type ligase e.g. Enterobacteria phage CC31 ligase.

In an embodiment of the invention, the ligase comprises or consists of any one of the following amino acid sequences: SEQ ID NOs: 10-28.

In an embodiment of the invention, the ligase is immobilised, e.g. on a bead.

### Oligonucleotides Used as Starting Materials

The oligonucleotides used as a starting material for the processes of the invention are herein described as being a “pool” and a definition thereof is provided above. The pool is a non-homogenous set of oligonucleotides. The oligonucleotides which form the pool will have been produced by other oligonucleotide production methods, e.g. solid phase or enzymatic synthesis, and will therefore likely contain impurities.

The pool is composed of segments of the product oligonucleotides, which are then joined together whilst assembled on the template. Each segment will be a non-homogeneous set with impurities of differing lengths and/or incorrectly incorporated residues.

### Producing the Segment Oligonucleotides Using Enzymes

1) ssLigase, e.g. RNA Ligase

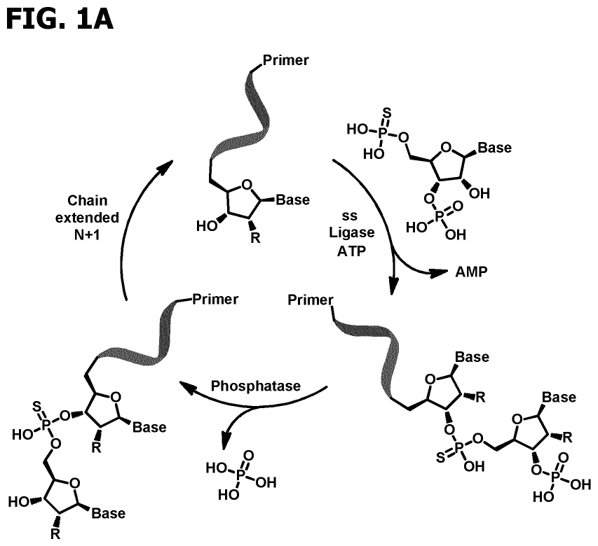

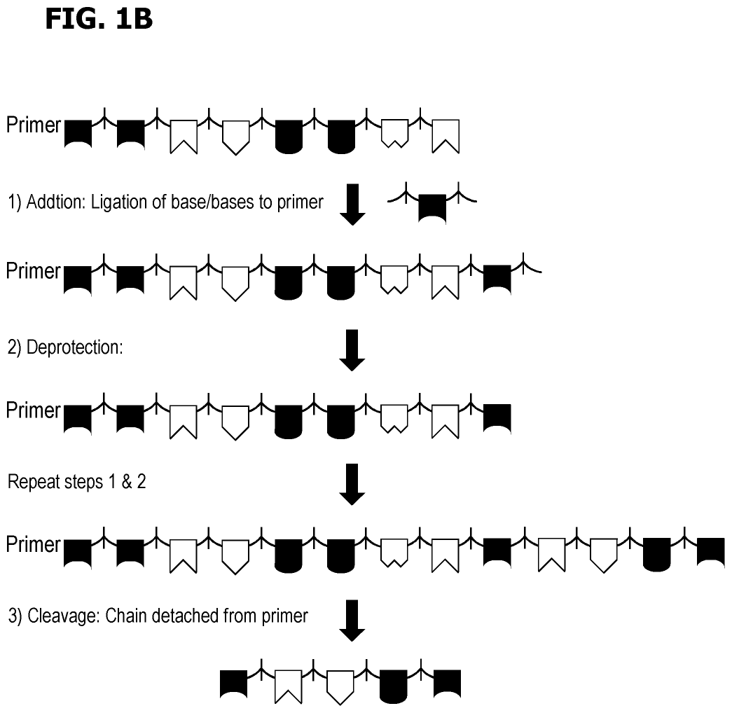

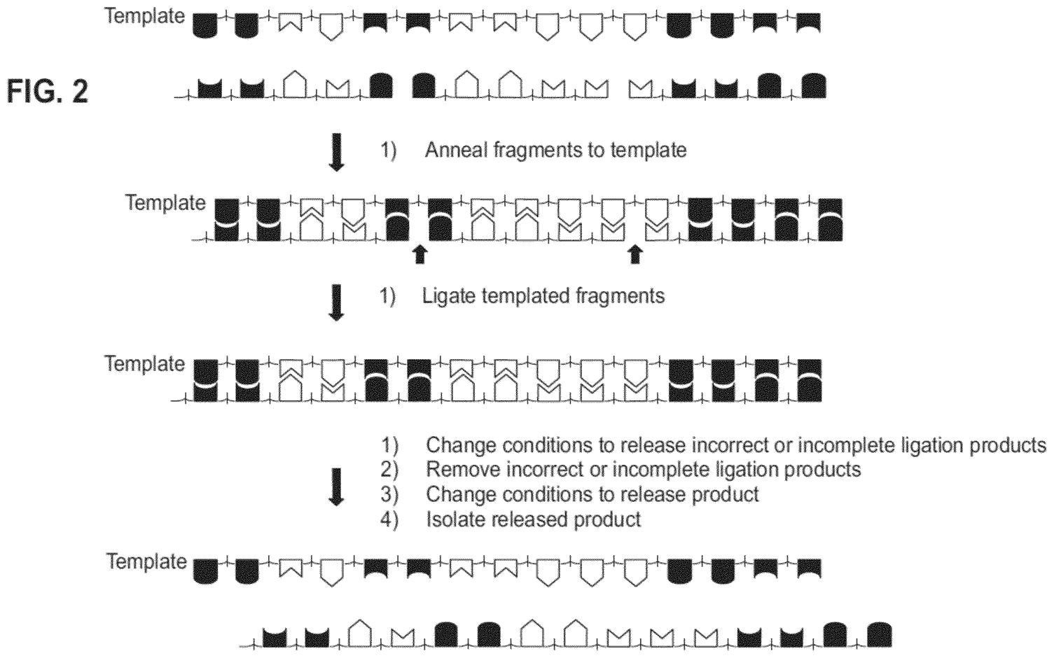

ssLigase catalyses the ATP driven addition of, for example, 3′,5′ nucleotide bisphosphates, 3′,5′ nucleotide thiophosphates (e.g. 3′,5′ bisthiophosphate or 3′-phosphate-5′-thiophosphate or 3′-thiophosphate-5′-phosphate) or 3′5′ nucleotide dithiophosphates (e.g. 3′,5′ bisdithiophosphate or 3′-phosphate-5′-dithiophosphate or 3′-dithiophosphate-5′-phosphate) to the 3′-OH of a short oligonucleotide (primer) in a template-independent manner. A skilled person would appreciate that diphosphates (or dithiophosphates) or triphosphates (or other oligophosphates where one or more oxygen atoms has been substituted by sulphur) at the 3′ position of the sugar moiety may also be used, although the additional phosphate (or thiophosphate) moieties are not required. An equivalently modified dinucleotide, trinucleotide or tetranucleotide may be used instead of the aforementioned individual nucleotides. The oligonucleotide primer is usually a minimum of three nucleotides long. The resulting oligonucleotide of this addition reaction is one nucleotide longer than the starting oligonucleotide (or two, three or four nucleotides longer than the starting oligonucleotide if a dinucleotide, trinucleotide or tetranucleotide is used, respectively). The new 3′ position is now phosphorylated. In order to add a subsequent nucleotide, the 3′ phosphate of the growing oligonucleotide is removed to generate a 3′-OH by hydrolysis. This hydrolysis is typically done using a phosphatase enzyme as shown in FIG. 1.

### 2) Transferase

Terminal deoxynucleotidyl transferase (TdT) enzymes catalyse the addition of 3′-protected nucleotide triphosphates, e.g. protected by a 3′-O-azidomethyl, 3′-aminoxy or 3′-O-allyl group, to the 3′-OH of a short oligonucleotide (primer) in a template-independent manner. This oligonucleotide primer is usually a minimum of three nucleotides long.

Suitable methods are set out, for example, in EP2796552, U.S. Pat. No. 8,808,989, WO16128731 A1 and WO16139477 A1.

The primer oligonucleotide used in the above described ssLigase and transferase methods for producing segment oligonucleotides can:
(1) be retained as part of the segment oligonucleotide if desired, or
(2) be cleaved from the product oligonucleotide to allow separation of the desired product and allow for the possibility of recycling to make further segment oligonucleotides. Cleavage of the primer from the segment oligonucleotide can be performed using a sequence specific nuclease and an appropriate design of primer and segment such that the cleavage is both effective and precise.

### Examples

### Abbreviations

OMe O-Methyl
MOE O-Methoxyethyl (DNA backbone) or Methoxyethyl (RNA backbone)
CBD Cellulose Binding Domain
HPLC high performance liquid chromatography
PBS phosphate buffered saline
HAA Hexylammonium acetate
SDS PAGE sodium dodecyl sulphate polyacrylamide gel electrophoresis
LCMS liquid chromatography mass spectrometry
PO phosphodiester
DTT dithiothreitol
Ni-NTA Nickel nitrilotriacetic acid
/3Phos/3′-Phosphate group
/5Phos/5′ Phosphate group
(p) phosphate group
PS or * phosphorothioate/ideoxyl
/ideoxyl/ 2′ deoxyInosine
5mC 5-Methylcytosine
BSA Bovine serum albumin
LNA locked nucleic acid
WT wild-type

### Example 1: Oligonucleotide (DNA) Segment Assembly and Ligation with Wild-Type T4 DNA Ligase

### 1.1 Chemical Synthesis of Starting and Control Sequences

In order to demonstrate that multiple short oligonucleotides (“segments”) could be assembled in the correct order on a complementary template strand and ligated to give the desired final product (“target”), the segments, target and template sequences, as detailed in Table 1, were chemically synthesised using standard methods.

### 1.2 HPLC Analysis

HPLC analysis was carried out using an Agilent ZORBAX Eclipse Plus XDB-C18 column (4.6×150 mm, 5 μm dp. Agilent P/N 993967-902) running at 0.2 ml/min while absorbance was monitored at 258 nm. The column was maintained at 60° C. 20 μl of sample was injected and a gradient from 20-31% buffer B was run over 20 minutes before being stepped up to 80% buffer B for 5 minutes.
Buffer A: 75 ml 1 M HAA, 300 ml isopropyl alcohol, 200 ml acetonitrile, 4425 ml water
Buffer B: 650 ml isopropyl alcohol, 350 ml acetonitrile

TABLE 1

| | | % HPLC | Amount |
|---|---|---|---|
| Name | Sequence | purity | (mg) |
| 5′-segment | 5′-GGC CAA-3′ | 100.0 | 21.6 |
| centre segment | 5′-(p)ACC TCG GC-3′ | 96.9 | 58.1 |
| 3′-segment | 5′-(p)T TAC CT-3′ | 98.8 | 39.8 |
| Target | 5′-GGC CAA ACC TCG GCT TAC CT-3′ | 98.4 | 101.7 |
| | (SEQ ID NO: 1) | | |
| Biotinylated template | 5′-biotin TT TAG GTA AGC CGA GGT | 96.9 | 130.7 |
| | TTG GCC-3′ (SEQ ID NO: 2) | | |

(p) phosphate

1.3 Oligonucleotide Assembly and Ligation Method with Commercial T4 DNA Ligase (SEQ ID NO:3)

The 5′ segment, centre segment and 3′ segment were assembled on the template: each segment and the template was dissolved in water at a concentration of 1 mg/ml and then mixed as follows.

| | | |
|---|---|---|
| 5′-segment | 2 | μl |
| centre segment | 2 | μl |
| 3-segment | 2 | μl |
| biotinylated template | 2 | μl |
| H₂O | 36 | μl |

The combined oligonucleotide solution was incubated at 94° C. for 5 minutes and cooled to 37° C. before incubating at 37° C. for a further 5 minutes to allow the segments to anneal to the template. 2 μl (equivalent to 2 μg) T4 DNA ligase (NEB) and 4 μl of 1×T4 DNA Ligation Buffer (NEB) were then added and the reaction (total reaction volume 50 μl) was incubated at room temperature for one hour. Following this, 40 μl of streptavidin coated magnetic beads were added and the suspension incubated at room temperature for 10 minutes to allow the biotinylated template to bind to the streptavidin beads. The streptavidin beads were washed with 2×100 μl PBS to remove unbound segments. The wash was analysed by HPLC. The reaction mixture was then incubated at 94° C. for 10 minutes to separate the bound ligation products (or any bound segments) from the template before being rapidly cooled on ice to ‘melt’ the DNA and stop reannealing of the oligonucleotides products (or segments) to the template. Analysis of the ligation reaction was then carried out by HPLC.

1.4 Oligonucleotide Assembly and Ligation Method with In-House T4 DNA Ligase Bead Slurry

### 1.4.1 Bead Slurry Generation

T4 ligase (SEQ ID NO:4) fused at the N-terminal to a cellulose binding domain (CBD) was produced using standard cloning, expression and extraction methods. This T4 ligase amino acid sequence differs from the commercial T4 ligase sequence (SEQ ID NO:3) in that the N-terminal methionine (M) has been replaced with glycine and serine (GS). These amino acid substitutions were done to aid the generation and expression of the CBD fusion protein. CBD-T4 ligase fusion protein was expressed in BL21 A1 cells (INVITROGEN). Supernatant was harvested and was added to 600 μl of PERLOZA 100 (PERLOZA) beads and shaken at 26° C. for 1 hour. The PERLOZA cellulose beads were then collected and washed with 2 ml buffer (50 mM Tris pH 8.0, 200 mM NaCl, 0.1% Tween 20, 10% Glycerol) followed by 5 ml PBS and were finally re-suspended in 200 μl PBS (10 mM PO₄³⁻, 137 mM NaCl, 2.7 mM KCl pH 7.4). In order to analyse protein expression, 15 μl of the PERLOZA bead slurry was mixed with 5 μl of SDS loading buffer and incubated at 80° C. for 10 minutes before being run on a SDS PAGE gradient gel (4-20%) according to a standard protocol.

### 1.4.2 Oligonucleotide Assembly and Ligation Using Bead Slurry

For T4 ligase bound to PERLOZA beads, the assembly and ligation method in 1.3 above was modified as follows. In the initial segment mixture, 36 μl of H₂O was reduced to 8 μl H₂O. After annealing, 2 μl of commercial T4 DNA ligase was replaced by 20 μl of PERLOZA bead slurry. Prior to adding the streptavidin magnetic beads, the PERLOZA beads were spun down and the supernatant removed. The streptavidin magnetic beads were added to the supernatant and incubated at room temperature for 10 minutes to allow the biotinylated template to bind to the streptavidin beads.

### 1.5 Results and Conclusions

The product, template and all three segment oligonucleotides were clearly resolved in the control chromatogram.

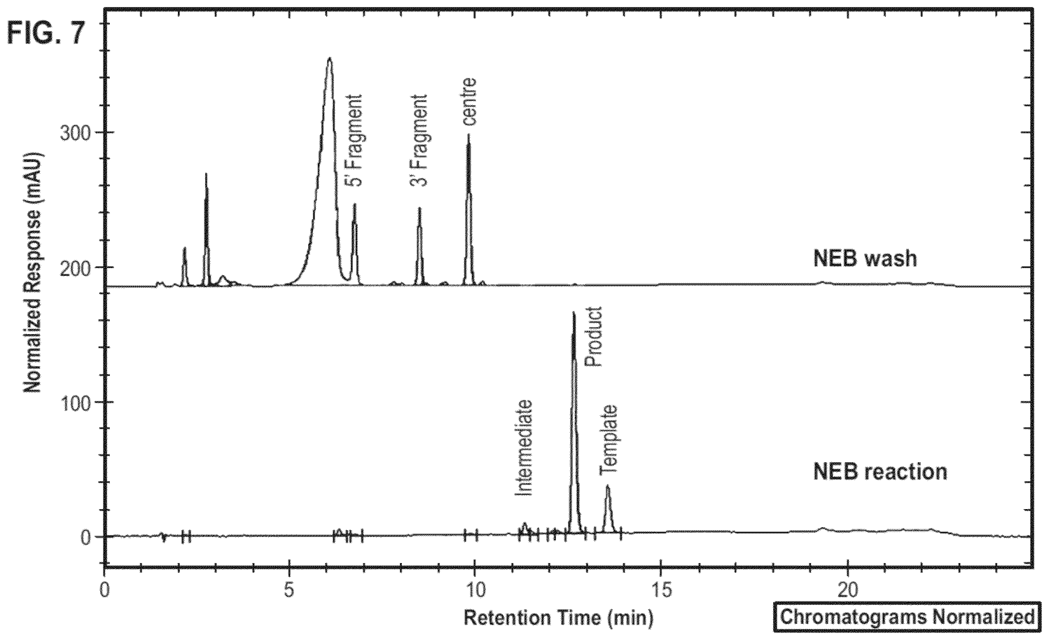

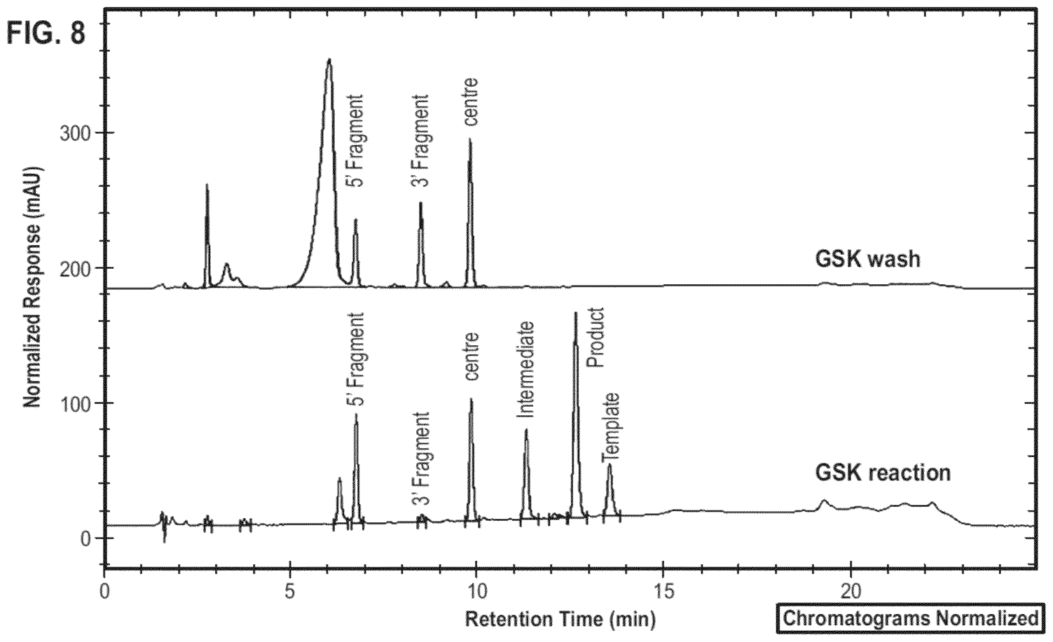

HPLC analysis of the ligase reactions showed that some unligated oligonucleotide segments remained, but commercial T4 DNA ligase (NEB) was able to catalyse ligation of the three segments to generate the desired product oligonucleotide (FIG. 7). The PERLOZA bead bound T4 DNA ligase appeared to be less efficient at ligation of the oligonucleotide segments, with oligonucleotide segments appearing in both the control wash sample and the reaction sample (FIG. 8). However, it is difficult to be sure whether the same amount of enzyme was added on the beads compared to the commercial enzyme, so a direct comparison of ligation efficiency was not possible.

### Example 2: 2′-OMe Ribose Modified Oligonucleotide Segment Assembly and Ligation with Wild-Type T4 DNA Ligase

### 2.1 2′ OMe at Each Nucleotide Position in Every Segment

In order to determine whether T4 DNA ligase was able to ligate oligonucleotide segments with modification at the 2′ position of the ribose ring, oligonucleotide segments were synthesized with the same sequence as for Example 1, but the 2′ position of the ribose ring was substituted with an OMe group and thymidine was replaced by uridine as shown below.

TABLE 2

| | | % HPLC | Amount |
|---|---|---|---|
| Name | Sequence | purity | (mg) |
| 5′-segment 2′-OMe | 5′-GGC CAA-3′ | 21 | 97.8 |
| centre segment 2′-OMe | 5′-(p)ACC UCG GC-3′ | 15.5 | 97.7 |
| 3′-segment 2′-OMe | 5′-(p)U UAC CU-3′ | 21.2 | 98.1 |
| Target 2′-OMe | 5′-GGC CAA ACC UCG GCU UAC CU-3′ | 88 | 96.9 |
| | (SEQ ID NO: 5) | | |

(p) = phosphate

Assembly, ligation and HPLC analysis were carried out using the methods of Example 1, with both commercial NEB ligase and T4 ligase CBD fusion bound to PERLOZA beads. The amount of water used in the reaction mix for the commercial T4 DNA ligase (NEB) experiment was 26 μl, rather than 36 μl, so that the final reaction volume was 40 μl. The amount of water used in the reaction mix for the in-house T4 DNA ligase bead slurry experiment was 23 μl, and the amount of beads used was 5 μl, so that the final reaction volume was also 40 μl. Control experiments using unmodified DNA as opposed to 2′-OMe DNA were run in parallel.

The results from the control experiments were in accordance with Example 1. No product was detected using HPLC for the 2′-OMe experiments indicating that T4 DNA ligase is unable to ligate fully 2′-OMe modified oligonucleotide segments regardless of whether a commercial T4 DNA ligase or in-house T4 DNA ligase CBD fusion bound to PERLOZA beads was used.

### 2.2 2′-OMe at Each Nucleotide Position in a Single Segment

Using a 1 mg/ml solution of each oligonucleotide the reactions as detailed in table 3 were set up.

TABLE 3

| | Experiment 2 | Experiment 3 | | | |
|---|---|---|---|---|---|
| Experiment 1 | (single 2′-OMe | (single 2′-OMe | Experiment 4 | Experiment 5 | Volume |
| (No ligase control) | segment - 3′) | segment - 5′) | (all 2′-OMe) | (all unmodified) | (μl) |
| template | template | template | template | template | 2 |
| 5′ segment | 5′ segment | 2′-OMe | 2′-OMe | 5′ segment | 2 |
| | | substituted | substituted | | |
| | | 5′ segment | 5′ segment | | |
| 3′ segment | 2′-OMe | 3′ segment | 2′-OMe | 3′ segment | 2 |
| | substituted | | substituted | | |
| | 3′ segment | | 3′ segment | | |
| Centre | Centre | Centre | 2′-OMe | Centre | 2 |
| segment | segment | segment | substituted | segment | |
| | | | Centre segment | | |
| H₂O | H₂O | H₂O | H₂O | H₂O | up to 40 total |

Assembly and ligation were carried out using the methods of Example 1 with commercial NEB ligase and in-house PERLOZA bound T4 DNA ligase.

Reactions were incubated at 94° C. for 5 minutes, followed by incubation for 5 minutes at 37° C. to allow for annealing. 4 μl of 1×NEB T4 DNA ligation buffer was added to each reaction along with 5 μl (approximately 2 μg) of in-house T4 DNA ligase or 2 μl (approximately 2 μg) commercial T4 DNA ligase (apart from Experiment 1 which was a no ligase control) and the ligation reaction was allowed to proceed for 2 hours at room temperature. Streptavidin magnetic beads were then added to each reaction and the reactions heated to 94° C. before rapid cooling on ice as described in Example 1 to separate the template from starting materials and products.

The processed reactions were split in two: half were analysed by HPLC as described for Example 1 (section 1.2). The other half of the sample was retained for mass spectrometry to confirm the HPLC results.

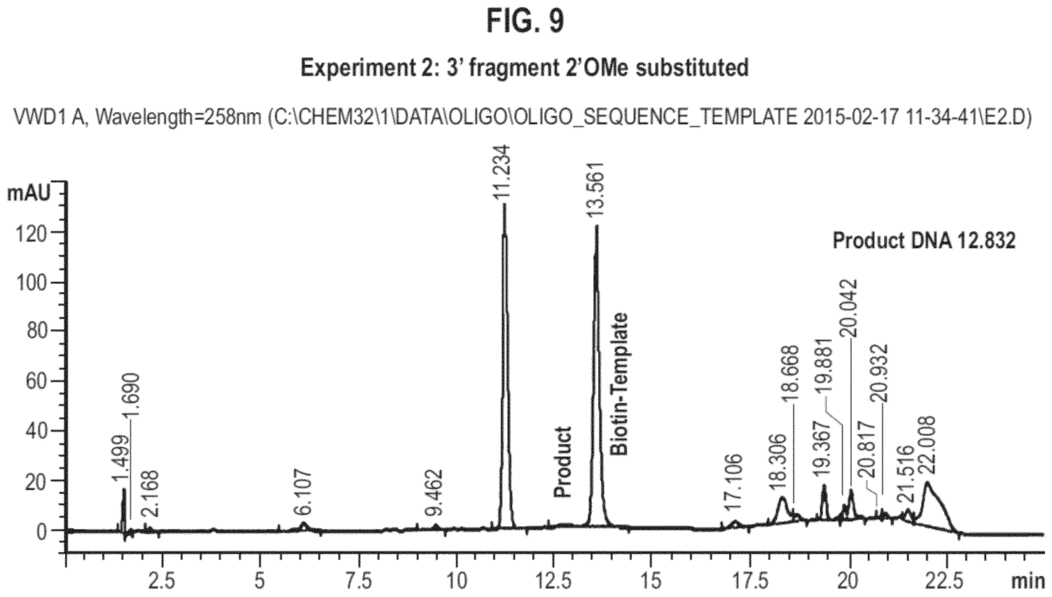

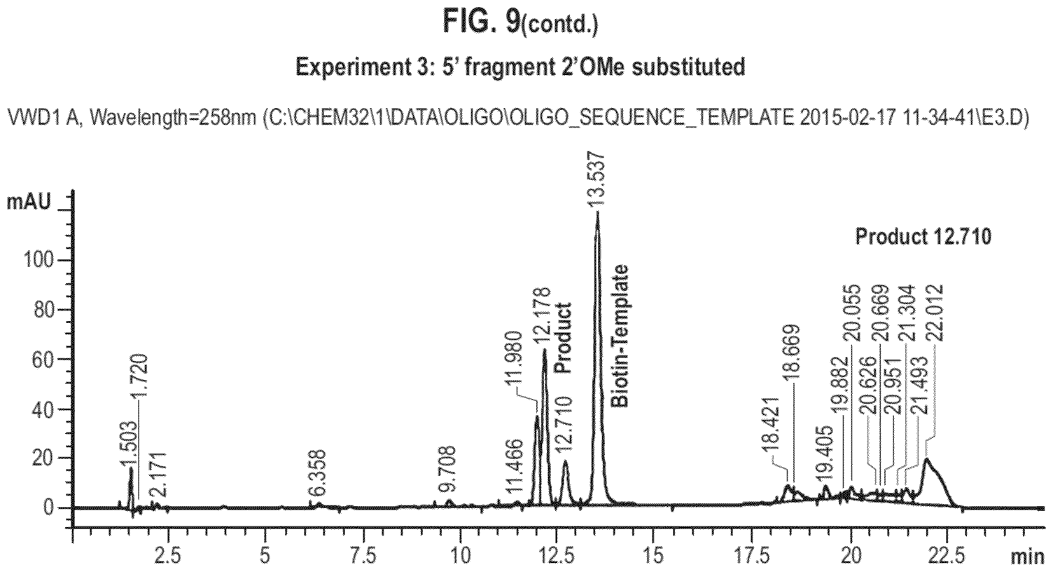

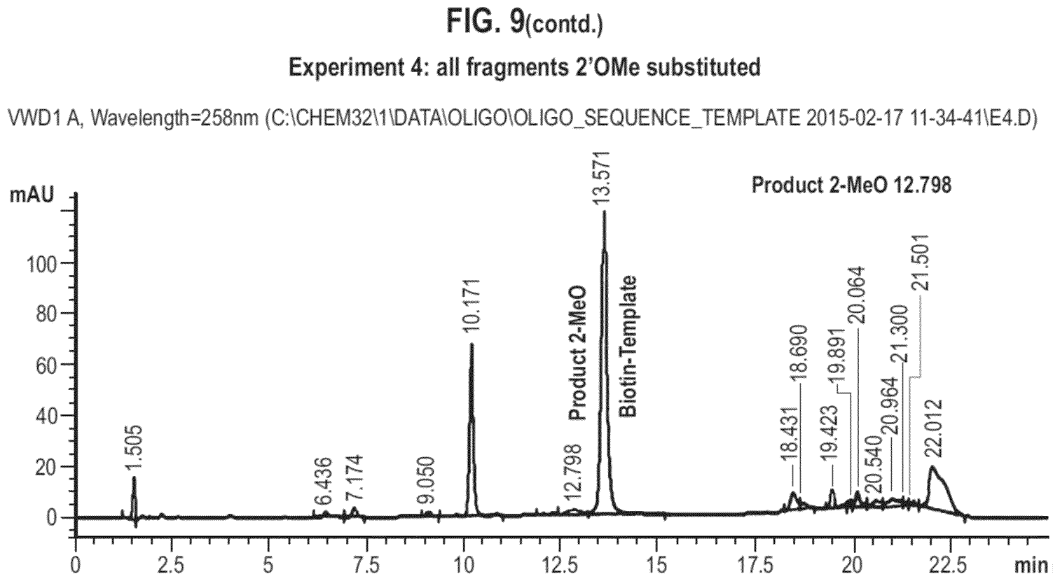

Ligation of unmodified oligonucleotide segments (experiment 5) proceeded as expected to produce full length product. A small amount of ligation was seen when the 5′ segment was 2′-OMe substituted (Experiment 3) as shown in FIG. 9, but no ligation was seen when the 3′ segment was 2′-OMe substituted (Experiment 2). No significant product was seen when all three segments were 2′-OMe substituted (Experiment 4) in accordance with 2.1.

### 2.3 Conclusion

Wild-type T4 DNA ligase is poor at ligating 2′-OMe substituted oligonucleotide segments, but is slightly less sensitive to modification of the 5′ oligonucleotide segment than the 3′ segment.

### Example 3: 2′-OMe Ribose Modified Oligonucleotide Segment Assembly and Ligation with Wild-Type and Mutant Ligases

### 3.1 Materials

Wild-type Enterobacteria phage ligase CC31 (SEQ ID NO:6), wild-type Shigella phage Shf125875 ligase (SEQ ID NO:8), and 10 mutant T4 ligases of SEQ ID NO: 10-19, each fused at the N-terminus to a CBD, were produced using standard cloning, expression and extraction methods. As disclosed in 1.4.1, in order to generate and express the CBD fusion proteins the N-terminal methionine (M) was replaced with glycine and serine (GS) in each case (e.g. SEQ ID NO:7 for Enterobacteria phage ligase CC31 and SEQ ID NO:9 for Shigella phage Shf125875 ligase).

The following oligonucleotides were synthesized by standard solid phase methods.

TABLE 4

| | | % HPLC | Amount |
|---|---|---|---|
| Name | Sequence | purity | (mg) |
| 5′-segment | 5′-(OMe)G(OMe)G(OMe)C(OMe)C(OMe)AA-3′ | 99.35 | 24.7 |
| centre segment | 5′-(p)ACC TCG GC-3′ | 96.9 | 58.1 |
| 3′ segment | 5′-(p)TTA CCT-3′ | 97.88 | 29.5 |
| Biotinylated | 5′-biotin TT TAG GTA AGC CGA GGT TTG | 96.9 | 130.7 |
| template | GCC-3′ (SEQ ID NO: 2) | | |

N.B. OMe indicates 2′ methoxy substitution on the ribose ring
(p) = phosphate
*note that the first 5 nucleotides are 2′-OMe modified (GGCCA), but the
final A is not

3.2 Oligonucleotide Assembly and Ligation Method with Ligase Bead Slurries

### 3.2.1 Bead Slurry Generation

Ligases fused to CBD were bound to PERLOZA beads as described in 1.4 to generate a bead slurry.

### 3.2.2 Oligonucleotide Assembly and Ligation Using Bead Slurry

Ligation reactions were prepared with the components below to a final volume of 50 μl in a 96 well plate:

| | | |
|---|---|---|
| 2 | μL | ~1 mg/mL 5′ (2′-OMe) segment |
| 2 | μL | ~1 mg/mL centre segment |
| 2 | μL | ~1 mg/mL 3′ segment |
| 2 | μL | ~1 mg/mL template |
| 5 | μL | NEB T4 DNA ligase buffer |
| 22 | μL | H₂O |
| 15 | μL | PERLOZA bound bead slurry |

The reaction was incubated for 15 minutes at room temperature prior to the addition of PERLOZA bead slurry to allow segments to anneal to the template. PERLOZA bead slurry was added and the reaction incubated at room temperature for 1 hour. After the hour incubation, the solution was transferred into an ACOPREP advance 350 filter plate (PN 8082) and the filter plate was placed on top of an ABGENE superplate (Thermo Scientific, #AB-2800) and centrifuged for 10 minutes at 4,000 rpm to remove the PERLOZA bead slurry. Solutions were then analysed by HPLC using the method described in Example 1 (section 1.2).

Each oligonucleotide assembly and ligation was repeated 6 times for each ligase.

### 3.3 Results and Conclusions

Wild-type Enterobacteria phage CC31 ligase (SEQ ID NO:6) and wild-type Shigella phage Shf125875 ligase (SEQ ID NO:8) are able to ligate a 2′ OMe substituted 5′ segment containing five 2′-OMe nucleobases and one deoxynucleobase to a segment containing only unmodified DNA. In addition, whilst wild-type T4 DNA ligase (SEQ ID NO:3 and 4) is poor at performing this reaction, as shown in Example 2 and reconfirmed here, a number of mutations at positions 368 and 371 confer the ability to ligate a 2′-OMe substituted 5′ segment containing five 2′ OMe nucleobases and one deoxynucleobase to a segment containing only unmodified DNA on the ligase (SEQ ID NO: 10-19).

### Example 4: 2′ MOE Ribose Modified and 5-Methyl Pyrimidine Modified Oligonucleotide Segment Assembly and Ligation with Mutant DNA Ligases

### 4.1 Materials

Modified oligonucleotide segments as set out in table 5 below were synthesised by standard solid phase-based methods.

TABLE 5

| | | | Mass | % |
|---|---|---|---|---|
| Segment | Sequence | MW | (mg) | purity |
| centre segment | 5′-(p)dCdCdTdCd | 2044.122 | 39 | 98.96 |
| | GdG-3′ | | | |
| MOE 3′-segment | 5′-(p)dCdTmTmAm | 2699.679 | 52 | 97.79 |
| | CmCmT-3′ | | | |
| MOE 5′-segment | 5′-mGmGmCmCmAdA | 2644.771 | 48 | 99.13 |
| | dA-3′ | | | |

(p) = phosphate, mX = MOE bases, dX = DNA bases
all 5-methyl pyrimidines

Mutant DNA ligases (SEQ ID NO:20-28) based upon wild-type Enterobacteria phage CC31 ligase, wild-type T4 ligase and wild-type Shigella phage Shf125875 ligase were each fused at the N-terminus to a cellulose binding domain (CBD), using standard cloning, expression and extraction methods. Ligases fused to CBD were bound to PERLOZA beads as described in 1.4 to generate a bead slurry. In order to release the ligases from the PERLOZA beads, 2 μl of TEV protease was added to the slurry and incubated overnight at 4° C. The cleaved protein, now lacking the cellulose binding domain, was collected by centrifugation for 10 min at 4000 rpm.

### 4.2 Method

The reaction was set up as follows:

| centre segment | 20 μM final | |
|---|---|---|
| MOE 3′ segment | 20 μM final | |
| MOE 5′ segment | 20 μM final | |
| Template | 20 μM final | |
| NEB T4 DNA ligase buffer | 5 | μl |
| mutant DNA ligase | 15 | μl |

| | |
|---|---|
| H₂O | To make final |
| | reaction 50 μl |

All components were mixed and vortexed prior to addition of DNA ligase. Reactions were incubated for 1 hour at 35° C. After 1-hour reactions were stopped by heating at 95° C. for 5 minutes in a PCR block.

Samples were analysed by both HPLC and LCMS to confirm product identity according to the HPLC protocol used in Example 1 (section 1.2). Controls of commercial NEB T4 DNA ligase and a negative control (H₂O instead of any ligase) were included.

### 4.3 Results and Conclusions

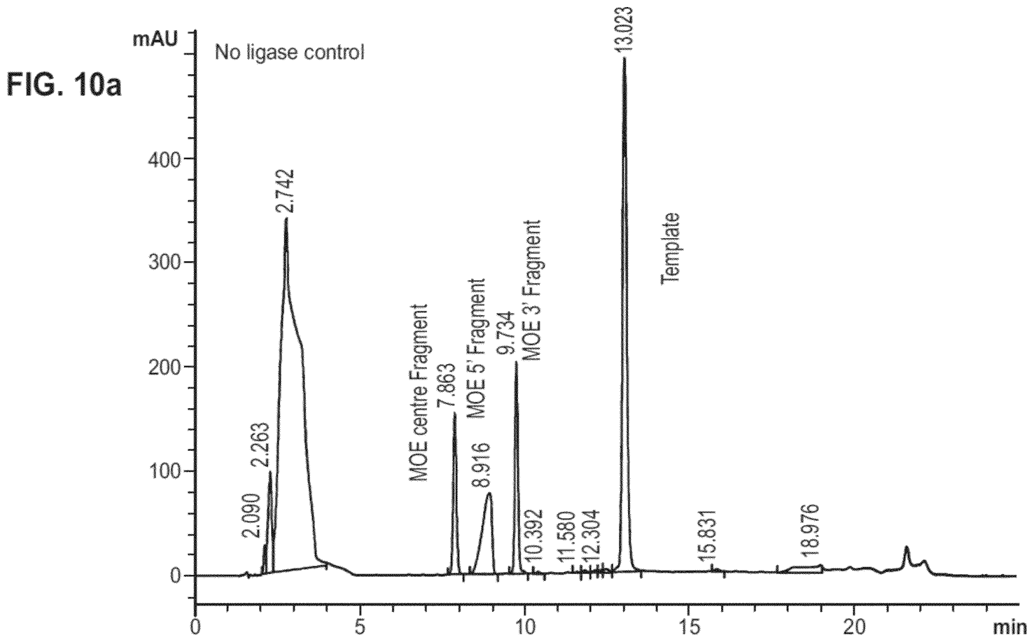

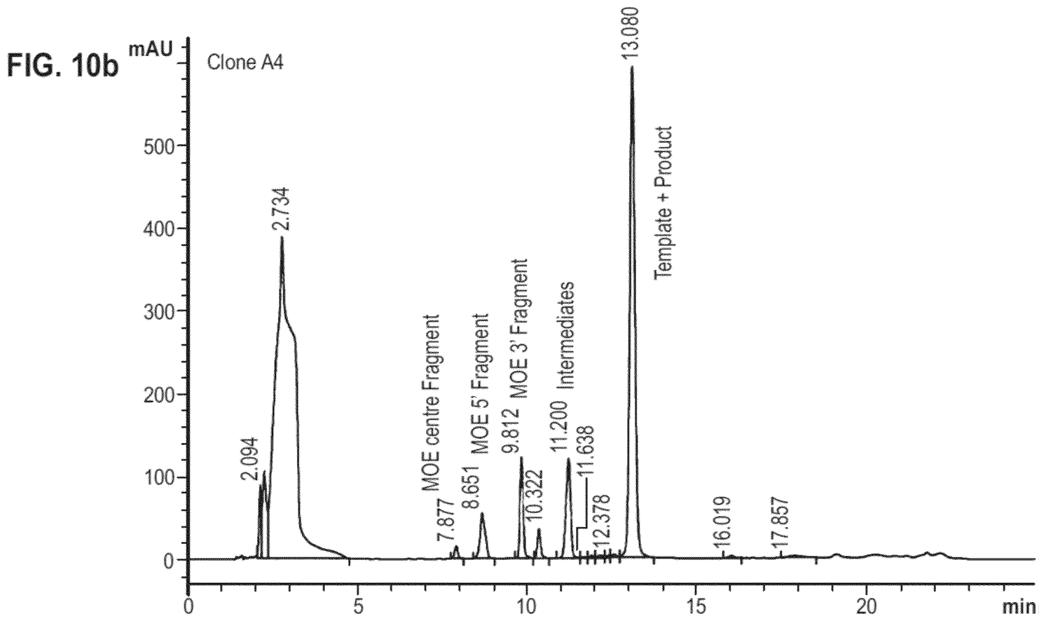

FIG. 10 shows the HPLC traces for the control reaction and the reaction catalysed by SEQ ID NO:23 (clone A4-mutant Enterobacteria phage CC31 ligase). The product and template co-elute using this HPLC method so product appears as an increase in the peak area of the product+template peak. In the mutant ligase trace (b) not only does the product+template peak increase but two new peaks appear at 10.3 and 11.2 minutes. These peaks correspond to the ligation of the centre segment with either the MOE 5′ segment or the MOE 3′ segment. Also, the input segment peaks are substantially smaller than the control in line with product and intermediate peaks increasing. The NEB commercial T4 ligase trace (a) showed a slight increase in the peak area for template+product, a small amount of intermediate ligation product along with a concomitant decrease in input oligonucleotide segments. The mutant ligase (SEQ ID NO:23), however, showed substantially greater product+template peak area and concomitant reduction in peak areas for input oligonucleotide segments. Thus, the mutant ligase (SEQ ID NO:23) is a much more effective ligase for the 2′ MOE substituted segments than commercial T4 DNA ligase. Similar improvements were shown for the other mutant ligases (SEQ ID NO:20, 21, 24-28).

### Example 5: Effect of Different Nucleotide Pairing at the Ligation Site

### 5.1 Materials

Mutant Enterobacteria phage CC31 ligase (SEQ ID NO:23) fused at the N-terminal to a cellulose binding domain (CBD) was produced using standard cloning, expression and extraction methods. Extracted CBD-mutant Enterobacteria phage CC31 ligase fusion protein was added to 25 ml of PERLOZA 100 (PERLOZA) cellulose beads and shaken at 20° C. for 1 hour. The PERLOZA beads were then collected and washed with 250 ml buffer (50 mM Tris pH 8.0, 200 mM NaCl, 0.1% Tween 20, 10% Glycerol) followed by 250 ml PBS and were finally re-suspended in 10 ml PBS (10 mM PO₄³⁻, 137 mM NaCl, 2.7 mM KCl pH 7.4). In order to analyse protein expression, 15 μl of the PERLOZA bead slurry was mixed with 5 μl of SDS loading buffer and incubated at 80° C. for 10 minutes before being run on a SDS PAGE gradient gel (4-20%) according to a standard protocol. For the release of the ligase from the beads, 70 μl of TEV protease was added and incubated overnight at 4° C. with shaking. Ligase was collected by washing the digested beads with 80 ml of PBS. The ligase was then concentrated down to 1.2 ml using an Amicon 30 Kd MCO filter.

The following biotinylated DNA template oligonucleotides (table 6) and DNA segment oligonucleotides (table 7) were synthesized by standard solid phase methods. Please note that the nucleotides in bold are the ones present at the ligation site (i.e. those nucleotides that were joined together in the ligation reaction-table 7; and those nucleotides that are complementary to those joined via the ligation reaction-table 6).

TABLE 6

| Template | Sequence (nucleotides complementary to junction | |
|---|---|---|
| number | nucleotides are in bold) | SEQ ID NO |
| 1 | 5′-biotin TTTGGTGCGAAGCAGACTGAGGC-3′ | 30 |
| 2 | 5′-biotin TTTGGTGCGAAGCAGAGTGAGGC-3′ | 31 |
| 3 | 5′-biotin TTTGGTGCGAAGCAGATTGAGGC-3′ | 32 |
| 4 | 5′-biotin TTTGGTGCGAAGCAGAATGAGGC-3′ | 33 |
| 5 | 5′-biotin TTTGGTGCGAAGCAGTCTGAGGC-3′ | 34 |
| 6 | 5′-biotin TTTGGTGCGAAGCAGTGTGAGGC-3′ | 35 |
| 7 | 5′-biotin TTTGGTGCGAAGCAGTTTGAGGC-3′ | 36 |
| 8 | 5′-biotin TTTGGTGCGAAGCAGTATGAGGC-3′ | 37 |
| 9 | 5′-biotin TTTGGTGCGAAGCAGCCTGAGGC-3′ | 38 |
| 10 | 5′-biotin TTTGGTGCGAAGCAGCGTGAGGC-3′ | 39 |
| 11 | 5′-biotin TTTGGTGCGAAGCAGCTTGAGGC-3′ | 40 |
| 12 | 5′-biotin TTTGGTGCGAAGCAGCATGAGGC-3′ | 41 |
| 13 | 5′-biotin TTTGGTGCGAAGCAGGCTGAGGC-3′ | 42 |
| 14 | 5′-biotin TTTGGTGCGAAGCAGGGTGAGGC-3′ | 43 |
| 15 | 5′-biotin TTTGGTGCGAAGCAGGTTGAGGC-3′ | 44 |
| 16 | 5′-biotin TTTGGTGCGAAGCAGGATGAGGC-3′ | 45 |

TABLE 7

| | | Sequence (junction |
|---|---|---|
| Segment | Identifier | nucleotides are in bold) |
| 5′ | A | 5′-GCCTCAG-3′ |
| 5′ | B | 5′-GCCTCAC-3′ |
| 5′ | C | 5′-GCCTCAA-3′ |
| 5′ | D | 5′-GCCTCAT-3′ |
| 3′ | E | 5′-(p)TCTGCT-3′ |
| 3′ | F | 5′-(p)ACTGCT-3′ |
| 3′ | G | 5′-(p)CCTGCT-3′ |
| 3′ | H | 5′-(p)GCTGCT-3′ |

(p) = phosphate

N.B. please note that, unlike previous examples, the ligation reactions in this example involve joining two segments together: a 5′-segment and a 3′-segment, i.e. there is no centre segment.

### 5.2 Method

Reactions were set up as follows:

TABLE 8

| Template | 5′-segment | 3′-segment |
|---|---|---|
| 1 | A | E |
| 2 | B | E |
| 3 | C | E |
| 4 | D | E |
| 5 | A | F |
| 6 | B | F |
| 7 | C | F |
| 8 | D | F |
| 9 | A | H |
| 10 | B | H |
| 11 | C | H |
| 12 | D | H |
| 13 | A | G |
| 14 | B | G |
| 15 | C | G |
| 16 | D | G |

For each 50 μL reaction:

| | | |
|---|---|---|
| 3′ segment (1 mM stock, 20 μM final) | 1 | μl |
| 5′ segment (1 mM stock, 20 μM final) | 1 | μl |
| Template (1 mM stock, 20 μM final) | 1 | μl |
| NEB DNA ligase buffer (for T4 ligase) | 5 | μl |
| Mutant CC31 DNA ligase (0.45 mM stock, 90 μM final) | 10 | μl |

| | |
|---|---|
| H₂O | up to 50 μl |

Each reaction mix was incubated at 35° C. both for 30 minutes and 1 hour. Each reaction was terminated by heating at 95° C. for 5 minutes. HPLC analysis was carried out.

### 5.3 Results and Conclusions

All of the reactions produced a product peak after 1 hour incubation in HPLC analysis. Accordingly, the ligation method works for all combinations of nucleotides at the junctions to be joined. Optimisation to improve product yield is possible, but was not necessary as the results were conclusive and it was clear that the reaction was working for all combinations of nucleotides at the junctions to be joined.

### Example 6: Effect of Different Modifications at the Ligation Site

### 6.1 Materials

Mutant Enterobacteria phage CC31 ligase (SEQ ID NO:23) was produced as described in 5.1 and Chlorella virus DNA ligase (SEQ ID NO:29, commercially available as SplintR ligase, NEB) was purchased.

The following biotinylated template oligonucleotide and segment oligonucleotides (table 9) were synthesized by standard solid phase methods.

TABLE 9

| | Sequence (junction |
|---|---|
| Name | modification in bold) |
| Template | 5′-biotin TTTAGGTAAGCCGAGGTT |
| | TGGCC-3′ |
| | (SEQ ID NO: 2) |
| 5′ segment (WT) | 5′-GGCCAAA-3′ |
| 5′ segment (Mo1) | 5′-GGCCAA(OMe)A-3′ |
| 5′ segment (Mo2) | 5′-GGCCAA(F)A-3′ |
| centre segment (WT) | 5′-(p)CCTCGG-3′ |
| centre segment (Mo4A) | 5′-(p)(OMe)CCTCGG-3′ |
| centre segment (Mo5A) | 5′-(p)(F)CCTCGG-3′ |
| centre segment (Mo7) | 5′(p)(Me)CCTCGG-3′ |
| 3′ segment (WT) | 5′-(p)CTTACCT-3′ |
| 3′ segment (Mo8) | 5′-(p)(Me)CTTACCT3′ |

OMe indicates 2′ methoxy substitution on the ribose ring
F indicates 2′ fluoro substitution on the ribose ring
All remaining sugar residues are deoxyribose residues
Me indicates 5-methyl cytosines

### 6.2 Method

Reactions were set up as follows:

TABLE 10

| | | Centre | |
|---|---|---|---|
| Reaction | 5′ segment | segment | 3′ segment |
| 1 | WT | WT | WT |
| 2 | WT | Mo4A | WT |
| 3 | WT | Mo5A | WT |
| 4 | WT | Mo7 | WT |
| 5 | Mo1 | WT | WT |
| 6 | Mo1 | Mo4A | WT |
| 7 | Mo1 | Mo5A | WT |
| 8 | Mo1 | Mo7 | WT |
| 9 | Mo2 | WT | WT |
| 10 | Mo2 | Mo4A | WT |
| 11 | Mo2 | Mo5A | WT |
| 12 | Mo2 | Mo7 | WT |
| 13 | WT | WT | Mo8 |
| 14 | WT | Mo4A | Mo8 |
| 15 | WT | Mo5A | Mo8 |
| 16 | WT | Mo7 | Mo8 |
| 17 | Mo1 | WT | Mo8 |
| 18 | Mo1 | Mo4A | Mo8 |
| 19 | Mo1 | Mo5A | Mo8 |
| 20 | Mo1 | Mo7 | Mo8 |
| 21 | Mo2 | WT | Mo8 |
| 22 | Mo2 | Mo4A | Mo8 |
| 23 | Mo2 | Mo5A | Mo8 |
| 24 | Mo2 | Mo7 | Mo8 |

For each 50 μL reaction:

| | | |
|---|---|---|
| 3′-segment (1 mM, stock, 20 μM final) | 1 | μl |
| centre segment (1 mM, stock, 20 μM final) | 1 | μl |
| 5′-segment (1 mM, stock, 20 μM final) | 1 | μl |
| Template (1 mM, stock, 20 μM final) | 1 | μl |

| | |
|---|---|
| H₂O | up to 50 μl |

For Mutant Enterobacteria phage CC31 ligase (SEQ ID NO:23)

| | | |
|---|---|---|
| DNA ligase buffer (50 mM Tris-HCl, 1 mM DTT) | 5 | μl |
| Mutant CC31 DNA ligase (0.45 mM) | 10 | μl |
| MnCl₂ (50 mM) | 5 | μl |
| ATP (10 mM) | 10 | μl |

Whereas for Chlorella virus DNA ligase (SEQ ID NO:29, commercially available as SplintR ligase, NEB)

| | |
|---|---|
| NEB DNA ligase buffer (for Chlorella) | 5 μl |
| Chlorella virus DNA ligase | 2 μl |

Each reaction mix was incubated at 20° C. 1 hour. Each reaction was terminated by heating at 95° C. for 10 minutes. HPLC analysis was carried out using the method of Example 1.

### 6.3 Results and Conclusions

All of the reactions produced a product peak in HPLC analysis. Accordingly, the ligation method works for all combinations of modifications tested at the junctions to be joined. Optimisation to improve product yield is possible, but was not necessary as the results were conclusive and it was clear that the reaction was working for all combinations of modifications tested at the junctions to be joined.

### Example 7: Ability to Use Different Numbers of Segments to Build Larger Oligonucleotides

### 7.1 Materials

Mutant Enterobacteria phage CC31 ligase (SEQ ID NO:23) was produced as described in 5.1.

The following biotinylated template DNA oligonucleotides and DNA segment oligonucleotides (table 11) were synthesized by standard solid phase methods.

TABLE 11

| Name | Sequence |
|---|---|
| Template | 5′-biotin TTTGGTGCGAAGCAGAAGGTA |
| | AGCCGAGGTTTGGCC-3′ |
| | (SEQ ID NO: 47) |
| 5′ segment (1) | 5′-GGCCAAA-3′ |
| centre segment (2) | 5′-(p)CCTCGG-3′ |
| centre segment/ | 5′-(p)TCTGCT-3′ |
| 3′segment (5) | |
| centre segment (3) | 5′-(p)CTTACCT-3′ |
| 3′ segment (4) | 5′-(p)TCGCACC-3′ |

(p) = phosphate,

### 7.2 Method

Reactions were set up as follows:

TABLE 12

| | Centre | | Total number |
|---|---|---|---|
| 5′ segment | segment(s) | 3′ segment | of segments |
| 1 | 2 and 3 | 5 | 4 |
| 1 | 2, 3 and 5 | 4 | 5 |

Reactions were run in phosphate buffered saline, pH=7.04 in a total volume of 100 μl and set up as follows:-
Template (20 μM final)
Each segment (20 μM final)
MgCl₂ (10 mM final)
ATP (100 μM final)
Mutant CC31 DNA ligase (25 μM final)

Each reaction was incubated at 28° C. overnight before being terminated by heating at 94° C. for 1 minute. Products were analysed by HPLC mass spec.

### 7.3 Results and Conclusions

The reaction using 4 segments produced a fully ligated product of 27 base pairs in length. The reaction using 5 segments produced a product of 33 base pairs in length. In both cases the observed mass of the product was in concordance with that expected for the desired sequence. In conclusion, it is clearly possible to assemble multiple segments to generate oligonucleotides of the desired length and sequence as defined by the appropriate complementary template sequence.

Example 8: Assembly and Ligation of 5-10-5 Segments to Form a Gapmer, Wherein the 5′ and the 3′ Segments Comprise (i) 2′-OMe Ribose Sugar Modifications, (ii) Phosphorothioate Linkages or (iii) 2′-OMe Ribose Sugar Modifications and Phosphorothioate Linkages; and Wherein the Central Segment is Unmodified DNA

### 8.1 Materials

Mutant Enterobacteria phage CC31 ligase (SEQ ID NO:23) was produced as described in 5.1 the following biotinylated template DNA oligonucleotide and segment oligonucleotides (table 13) were synthesized by standard solid phase methods.

TABLE 13

| Name | Sequence |
|---|---|
| Template | 5′-biotin TTTGGTGCGAAGCAGACTGAGGC-3′ |
| | (SEQ ID NO: 30) |
| 3′ segment (30Me) | 5′-(p)(OMe)G(OMe)C(OMe)C(OMe)T(OMe)C-3′ |
| 3′ segment (3PS + OMe) | 5′-(p)(OMe)G*(OMe)C*(OMe)C*(OMe)T*(OMe)C-3′ |
| 3′ segment (3PS) | 5′-(p)G*C*C*T*C-3′ |
| centre segment (D) | 5′-(p)AGTCTGCTTC-3′ |
| 5′ segment (50Me) | 5′-(OMe)G(OMe)C(OMe)A(OMe)C(OMe)C-3′ |
| 5′ segment (5PS+OMe) | 5′-(OMe)G*(OMe)C*(OMe)A*(OMe)C*(OMe)C-3′ |
| 5′ segment (5PS) | 5′-G*C*A*C*C-3′ |

OMe indicates 2′ methoxy substitution on the ribose ring
All remaining sugar residues are deoxyribose residues
*phosphorothioate

### 8.2 Method

Reactions were set up as follows:

TABLE 14

| | | Centre | |
|---|---|---|---|
| Reaction | 3′ segment | segment(s) | 5′ segment |
| 1 | 3PS | D | 5PS |
| 2 | 3OMe | D | 5OMe |
| 3 | 3PS + OMe | D | 5PS + OMe |

Each of reactions 1, 2 and 3 were set up in 100 μl final volume in phosphate buffered saline with the following components:-

| | |
|---|---|
| 3′ segment | 20 μM final |
| Centre segment | 20 μM final |
| 5′ segment | 20 μM final |
| Template | 20 μM final |
| MgCl₂ | 10 mM final |
| ATP | 50 μM final |
| Enzyme | 25 μM final |

Each reaction mix was incubated at 20° C. overnight. Each reaction was terminated by heating at 95° C. for 10 minutes. HPLC mass spec analysis was carried out.

### 8.3 Results and Conclusions

Product oligonucleotide corresponding to the successful ligation of all three fragments was produced in all three reactions.

Accordingly, mutant Enterobacteria phage CC31 ligase (SEQ ID NO:23) is able to ligate 3 segments together to form a ‘gapmer’ where the 5′ and 3′ ‘wings’ have a phosphorothioate backbone, whereas the central region has a phosphodiester backbone, and all the sugar residues in the gapmer are deoxyribose residues. Enterobacteria phage CC31 ligase (SEQ ID NO:23) is also able to ligate 3 segments together to form a ‘gapmer’ where the 5′ and 3′ ‘wings’ have 2′-methoxyribose (2′-OMe) residues, whereas the central region has deoxyribose residues, and all of the linkages are phosphodiester linkages. Finally, Enterobacteria phage CC31 ligase (SEQ ID NO:23) is able to ligate 3 segments together to form a ‘gapmer’ where the 5′ and 3″wings' have the combined modifications (a phosphorothioate backbone and 2′-methoxyribose residues), whereas the central region has deoxyribose residues and phosphodiester linkages.

### Example 9: Assembly and Ligation of Segments Comprising Locked Nucleic Acids (LNA)

### 9.1 Materials

Mutant Enterobacteria phage CC31 ligase (SEQ ID NO:23) was produced as described in 5.1. A mutant Staphylococcus aureus NAD dependent ligase (NAD-14) was produced as described in 13.1 The following biotinylated template DNA oligonucleotide and segment oligonucleotides (table 15) were synthesized by standard solid phase methods.

TABLE 15

| Name | Sequence |
|---|---|
| Template | 5′-biotin TTTGGTGCGAA |
| | GCAGACTGAGGC-3′ |
| | (SEQ ID NO: 30) |
| 5′-segment | 5′- GCCTCAG-3′ |
| LNA 5′-segment (oligo 1) | 5′-GCCTCA(LNA)G-3′ |
| Centre segment | 5′-(p)TCTGCT-3′ |
| LNA centre segment (oligo 2) | 5′-(p) (LNA)TCTGCT-3′ |

(p) = phosphate
LNA = locked nucleic acid

### 9.2 Method

Reactions were set up as follows:

Reaction volume 100 μl

| | |
|---|---|
| Template | 20 μM final |
| Enzyme | 25 μM final |
| All oligonucleotide segments | 20 μM final |

Reactions were set up varying enzyme (mutant Enterobacteria phage CC31 ligase (SEQ ID NO:23) or NAD-14), divalent cation (Mg²⁺ or Mn²⁺) and combinations of oligonucleotide segments as set out in Table 16.

TABLE 16

| | SEQ ID | SEQ ID | | |
|---|---|---|---|---|
| Enzyme | NO: 23 | NO: 23 | NAD-14 | NAD-14 |
| Divalent | 10 mM | 10 mM | 10 mM | 10 mM |
| cation | MgCl₂ | MnCl₂ | MgCl₂ | MnCl₂ |
| Cofactor | 100 μM | 100 μM | 100 μM | 100 μM |
| | ATP | ATP | NAD | NAD |
| Buffer | PBS, | PBS, | 50 mM | 50 mM |
| | pH = 7.04 | pH = 7.04 | KH₂PO₄, | KH₂PO₄, |
| | | | pH 7.5 | pH 7.5 |
| 5′ segment + | Product | Product | Product | Product |
| Centre | | | | |
| segment | | | | |
| Oligo 1 + | Product | Product | Product | Product |
| Centre | | | | |
| segment | | | | |
| 5′ segment + | Product | Product | Product | Product |
| Oligo 2 | | | | |
| Oligo 1 + | No | No | No | No |
| oligo 2 | product | product | product | product |

Each reaction mix was incubated at 28° C. overnight. Each reaction was terminated by heating at 94° C. for 1 minute. HPLC mass spec. analysis was carried out.

### 9.3 Results and Conclusions

Product oligonucleotide was produced in control reaction reactions (unmodified oligonucleotides only) and where a single locked nucleic acid was included in one segment at the ligation junction regardless of whether it was on the 3′ or 5′ side of the junction. When locked nucleic acids were included at both sides (oligo 1+oligo 2) no product was detected. The data was similar for both enzymes and regardless of whether Mg2+ or Mn2+were used.

Enzyme mutations and/or selection screens could be carried out to identify an enzyme capable of ligating segments with a locked nucleic acid at both the 3′ and 5′ side of the junction.

Example 10: Assembly and Ligation of Three Segments (7-6-7) to Form a Gapmer Wherein the 5′ and the 3′ Segments Comprise 2′ MOE Ribose Sugar Modifications and all Linkages are Phosphorothioate Linkages, Using a Variant of Enterobacteria Phage CC31 Ligase in the Presence of Mg²⁺ or Mn²⁺

### 10.1 Phosphorothioate Bond Formation

In order to determine whether a mutant Enterobacteria phage CC31 ligase (SEQ ID NO:23) was able to ligate modified oligonucleotide segments with a phosphorothioate backbone, 2′ MOE ribose sugar modifications and 5-methylated pyrimidine bases, reactions were performed using the oligonucleotide segments shown in table 15. Reactions were performed in the presence of Mg2+ and Mn2+ ions.

### 10.2 Materials

Oligonucleotides were chemically synthesised using standard methods as shown below:

TABLE 17

| Name | Sequence |
|---|---|
| 5′-segment 2′- | 5′-mG*mG*mC*mC*mA*dA*dA-3′ |
| MOE PS | |
| centre segment | 5′-(p)*dC*dC*dT*dC*dG*dG -3′ |
| PS | |
| 3′-segment 2′- | 5′-(p)*dC*dT*mU*mA*mC*mC*mU-3′ |
| MOE PS | |
| Biotinylated | 5′-biotin dT dT dT dA dG dG dT dA |
| template | dA dG dC dC dG dA dG dG dT dT dT |
| | dG dG dC dC-3′ |
| | (SEQ ID NO: 2) |

(p)* = 5′-phosphorothioate, * = phosphorothioate
linkage, mX = MOE bases, dX = DNA bases all segments
and product have 5-methyl pyrimidines (with the
exception of the template)
mT and m (Me)U are considered to be equivalent

N.B. the target 2′MOE PS molecule produced by ligation of the segments in table 17, when hybridised to the biotinylated template shown in table 17, is:

5′-mG*mG*mC*mC*mA*dA*dA*dC*dC*dT*dC*dG*dG*dC*dT*mU*mA*mC*mC*mU-3′ (SEQ ID NO:1)

Purified mutant Enterobacteria phage CC31 ligase (SEQ ID NO:23) was prepared as described in example 5.1. HPLC analysis was carried out.

10.3 Oligonucleotide Assembly and Ligation Method with Enterobacteria Phone CC31 Ligase Variant (SEO ID NO:23)

Reactions were prepared as follows:

### MgCl₂ Reaction

| | |
|---|---|
| 10 × T4 DNA ligase buffer (NEB)* | 5 μl |
| template | 20 μM final |
| | concentration |
| 5′ segment 2′ MOE PS | 20 μM final |
| | concentration |
| centre segment PS | 20 μM final |
| | concentration |
| 3′ segment 2′ MOE PS | 20 μM final |
| | concentration |
| ligase (24.3 μM) | 10 μl |
| water | made up to 50 μL |

*1 × buffer contains 50 mM Tris-HCl, 10 mM MgCl₂, 1 mM ATP, 10 mM DTT, pH 7.5

### MnCl₂ Reaction

| | |
|---|---|
| 10 × ligase buffer* | 5 μl |
| ATP (10 mM) | 5 μl |
| MnCl₂ (50 mM) | 5 μl |
| template | 20 μM final |
| | concentration |
| 5′ segment 2′ MOE PS | 20 μM final |
| | concentration |
| centre segment PS | 20 μM final |
| | concentration |
| 3′ segment 2′ MOE PS | 20 μM final |
| | concentration |
| ligase (24.3 μM) | 10 μl |
| water | made up to 50 μL |

*1 × buffer contains 50 mM Tris-HCl, 10 mM DTT, pH 7.5

The final reactions contained 20 μM of each segment and template, 5 mM MgCl₂ or 5 mM MnCl₂, 1 mM ATP, 50 mM Tris-HCl, 10 mM DTT, pH 7.5 and 4.9 μM ligase. Additional reactions were prepared containing no enzyme and served as a negative control. Reactions were incubated for 16 hours at 25° C. and then quenched by heating to 95° C. for 5 minutes. Precipitated proteins were cleared by centrifugation and samples were analysed by HPLC.

### 10.4 Results and Conclusion

Product, template and segment oligonucleotides were clearly resolved in the control chromatogram and no ligation was observed. Ligase reactions performed in the presence of 5 mM MgCl₂ led to the formation of an intermediate product formed from the ligation of the 5′ segment and centre segments, but no full-length product was detected. Ligase reactions performed in the presence of MnCl₂ produced both full length product and intermediate (5′ segment plus centre segment intermediate). Both ligase reactions showed that unligated oligonucleotide segments remained. However, optimisation of the protocol is possible in order to maximise product yield.

Example 11: Assembly and Ligation of Three Segments (7-6-7) to Form a Gapmer Wherein the 5′ and the 3′ Segments Comprise 2′ MOE Ribose Sugar Modifications and all Linkages are Phosphorothioate Linkages, Using Wild-Type Chlorella Virus DNA Ligase in the Presence of Native Mg²⁺

### 11.1 Materials

In order to determine whether Chlorella virus DNA ligase (SEQ ID NO:29, commercially available as SplintR ligase, NEB) was able to ligate modified oligonucleotide segments with a phosphorothioate backbone, 2′ MOE ribose sugar modifications and 5-methylated pyrimidine bases reactions were performed using the oligonucleotide segments shown in example 10.2 table 17. Reactions were performed at 25° C., 30° C. and 37° C. to investigate the effect of temperature on the enzyme activity.

11.2 Oligonucleotide Assembly and Ligation Method with Commercial Chlorella Virus DNA Ligase (SEQ ID NO:29)

Each Oligonucleotide segment and template were dissolved in nuclease free water as detailed below:

| | |
|---|---|
| Biotinylated template | 249.6 ng/μl |
| 5′ segment 2′ MOE PS | 182.0 ng/μl |
| Centre segment PS | 534.0 ng/μl |
| 3′ segment 2′ MOE PS | 531.0 ng/μl |

Reactions were prepared as follows:

| | | |
|---|---|---|
| 10× buffer (NEB)* | 6 | μl |
| template | 3.8 | μl |
| 5′ segment 2′ MOE PS | 18.1 | μl |
| centre segment PS | 4.8 | μl |
| 3′ segment 2′ MOE PS | 6.3 | μl |
| water | 15 | μl |
| SplintR ligase (25 U/μl) | 6 | μl |

*1× buffer contains 50 mM Tris-HCl, 10 mM MgCl₂, 1 mM ATP, 10 mM DTT, pH 7.5

The final reactions contained 20 μM of each segment and template, 50 mM Tris-HCl, 10 mM MgCl₂, 1 mM ATP, 10 mM DTT, pH 7.5 and 2.5 U/μl ligase. Reactions were incubated at 25° C., 30° C. and 37° C. Additional reactions were prepared containing no enzyme and served as a negative control. Following 16 hours incubation, reactions were quenched by heating to 95° C. for 10 minutes. Precipitated proteins were cleared by centrifugation and samples were analysed by HPLC.

### 11.3 Results and Conclusion

Product, template and segment oligonucleotides were clearly resolved in the control chromatogram and no ligation was observed. HPLC analysis of the ligase reactions showed that unligated oligonucleotide segments remained, but Chlorella virus DNA ligase was able to successfully ligate the segments. The activity of the ligase increased with increasing temperature. At 25° C. the Chlorella virus DNA ligase was able to successfully ligate the 5′ segment and centre segment but no full-length product was observed. At 30° C. and 37° C., full length product was detected in addition to the intermediate formed from 5′ segment and centre segment.

Example 12: Screening a Panel of 15 ATP and NAD Ligases for Activity Towards the Ligation of Three Segments (7-6-7) to Form a Gapmer Wherein the 5′ and the 3′ Segments Comprise 2′ MOE Ribose Sugar Modifications and all Linkages are Phosphorothioate Linkages

### 12.1 Materials

Wild-type ATP and NAD dependent ligases described in table 18 and 19 were each fused at the N-terminus to a CBD. Genes were synthesised, cloned into pET28a and expressed in E. coli BL21(DE3) using standard cloning, expression and extraction methods.

TABLE 18
ATP dependent Ligases

| Name | Origin | SEQ ID |
|---|---|---|
| M1I5D1_Pbcv | Paramecium bursaria Chlorella virus NE-JV-4 | SEQ ID NO: 48 |
| M1I998_Pbcv | Paramecium bursaria Chlorella virus NYs1 | SEQ ID NO: 49 |
| M1HX09_Pbcv | Paramecium bursaria Chlorella virus NE-JV-1 | SEQ ID NO: 50 |
| M1HULO_Atcv | Acanthocystis turfacea Chlorella virus Canal-1 | SEQ ID NO: 51 |
| M1HRK1_Atcv | Acanthocystis turfacea Chlorella virus Br0604L | SEQ ID NO: 52 |
| M1I273_Atcv | Acanthocystis turfacea Chlorella virus NE-JV-2 | SEQ ID NO: 53 |
| M1I600_Atcv | Acanthocystis turfacea Chlorella virus TN603.4.2 | SEQ ID NO: 54 |
| M1H4A4_Atcv | Acanthocystis turfacea Chlorella virus GM0701.1 | SEQ ID NO: 55 |
| F5B464_Sphage | Synechococcus phage S-CRM01 | SEQ ID NO: 56 |
| A0A0F9M1S3 | marine sediment metagenome - uncharacterized protein | SEQ ID NO: 57 |

TABLE 19
NAD dependent ligases

| Name | Origin | SEQ ID |
|---|---|---|
| MtNAD | Mycobacterium tuberculosis (strain ATCC 25618/H37Rv) | SEQ ID NO: 58 |
| EfNAD | Enterococcus faecalis (strain ATCC 700802/V583) | SEQ ID NO: 59 |
| HiNAD | Haemophilus influenzae (strain ATCC 51907/DSM 11121/ | SEQ ID NO: 60 |
| | KW20/Rd) | |
| SaNAD | Staphylococcus aureus | SEQ ID NO: 61 |
| SpNAD | Streptococcus pneumoniae (strain P1031) | SEQ ID NO: 62 |

CBD-Ligase fusions were bound to PERLOZA beads as described in 1.4 with the following modifications. CBD-ligase fusion proteins were grown from a single colony of BL21(DE3) cells (NEB) and grown in a 50 mL expression culture. The cells were harvested by centrifugation, resuspended in 5-10 mL Tris-HCl (50 mM, pH 7.5) and lysed by sonication. The lysate was cleared by centrifugation and 1 mL of PERLOZA 100 (PERLOZA) beads (50% slurry, pre-equilibrated with 50 mM Tris-HCl pH 7.5) was added to the supernatant which was shaken at 20° C. for 1 hour. The PERLOZA cellulose beads were then collected and washed with 30 ml buffer (50 mM Tris pH 8.0, 200 mM NaCl, 0.1% Tween 20, 10% Glycerol) followed by 10 ml Tris-HCl (50 mM, pH 7.5) and were finally re-suspended in 1 mL Tris-HCl (50 mM, pH 7.5). In order to analyse protein expression, 20 μl of the PERLOZA bead slurry was mixed with 20 μl of SDS loading buffer and incubated at 95° C. for 5 minutes before being run on a SDS PAGE gradient gel (4-20%) according to a standard protocol.

### 12.2 2′ MOE and Phosphorothioate Modified Oligonucleotide Assembly and Ligation Method

Modified oligonucleotide segments with a phosphorothioate backbone, 2′ MOE ribose sugar modifications and 5-methylated pyrimidine bases shown in example 10.2 table 17 were used. Each oligonucleotide segment and template was dissolved in nuclease free water as detailed below:

| | | |
|---|---|---|
| Biotinylated template | 1500 | ng/μl |
| 5′ segment 2′ MOE PS | 1008 | ng/μl |
| Centre segment PS | 725 | ng/μl |
| 3′ segment 2′ MOE PS | 1112 | ng/μl |

ATP Assay mix was prepared as follows:

| | | |
|---|---|---|
| template | 85.6 | μl |
| 5′ segment 2′ MOE PS | 43.5 | μl |
| centre segment PS | 46.8 | μl |
| 3′ segment 2′ MOE PS | 40.1 | μl |
| DTT 1M | 8 | μl |
| MgCL2 1M | 4 | μl |
| ATP (50 mM) | 16 | μl |
| Tris 0.5M | 80 | μl |
| water | 476 | μl |

NAD Assay mix was prepared as follows:

| | | |
|---|---|---|
| template | 85.6 | μl |
| 5′ segment 2′ MOE PS | 43.5 | μl |
| centre segment PS | 46.8 | μl |
| 3′ segment 2′ MOE PS | 40.1 | μl |
| DTT 1M | 8 | μl |
| MgCL2 1M | 4 | μl |
| NAD (50 mM) | 1.6 | μl |
| Tris 0.5M | 80 | μl |
| water | 490.4 | μl |

Each immobilized protein (40 μl, 50% PERLOZA bead slurry) was pipetted into a PCR tube. The beads were pelleted by centrifugation and the supernatant was removed by pipetting. Assay mix (40 μl) was added to each reaction (The final reactions contained 20 μM of each segment and template, 50 mM Tris-HCl, 10 mM MgCl₂, 1 mM ATP or 100 μM NAD, 10 mM DTT, PH 7.5, and 40 μl of ligase on PERLOZA beads). A reaction containing no protein served as a negative control. Reactions were incubated for 18 hours at 30° C. and then quenched by heating to 95° C. for 10 minutes. Precipitated proteins were cleared by centrifugation and samples were analysed by HPLC.

### 12.3 Results and Conclusion

The product, template and segment oligonucleotides were clearly resolved in the control chromatogram and no ligation was observed. HPLC analysis of the ligase reactions showed that all proteins catalyzed the successful ligation of the 5′ segment and centre segment to form an intermediate product, but only some ligases catalyzed the ligation of all three segments to yield the full-length product as described in table 20. The NAD dependent ligase from Staphylococcus aureus (SaNAD, SEQ ID NO:61) yielded the most full length product. Optimisation to improve product yield is possible and within the skilled person's skill set.

TABLE 20

| | | Conversion (%)* | |
|---|---|---|---|
| Gene name | SED ID NO | intermediate | product |
| M1I5D1_Pbcv | 48 | 5.0 | 0.8 |
| M1I998_Pbcv | 49 | 5.1 | 0.0 |
| M1HX09_Pbcv | 50 | 14.4 | 6.6 |
| M1HULO_Atcv | 51 | 9.5 | 2.0 |
| M1HRK1_Atcv | 52 | 2.8 | 9.4 |
| M1I273_Atcv | 53 | 12.0 | 6.4 |
| M1I600_Atcv | 54 | 8.8 | 5.1 |
| M1H4A4_Atcv | 55 | 2.1 | 0.0 |
| F5B464_Sphage | 56 | 2.1 | 0.0 |
| A0A0F9M1S3_ms_metagenome | 57 | 1.4 | 0.0 |
| MtNAD | 58 | 7.1 | 0.0 |
| EfNAD | 59 | 19.1 | 0.0 |
| HiNAD | 60 | 0.3 | 0.0 |
| SaNAD | 61 | 10.5 | 11.8 |
| SpNAD | 62 | 19.0 | 0.0 |

*Conversion was calculated from the HPLC peak area relative to the template which is not consumed in the reaction and serves as an internal standard. Conversion = product area/(template + product area)*100

### Example 13: Semi-Continuous Ligation Reaction

### 13.1 Materials

A mutant Staphylococcus aureus ligase (NAD-14) fused at the N-terminal to a CBD was produced using standard cloning, expression and extraction methods. CBD-NAD-14 mutant ligase was then bound to PERLOZA beads: 50 ml of protein lysate was added to 7.5 ml PERLOZA beads, incubated at room temperature for 1 hour and then the beads were collected in a glass column (BioRad Econo-Column 10 cm length, 2.5 cm diameter #7372512). The beads were washed with 200 ml Buffer Y (50 mM Tris8, 500 mM NaCl, 0.1% Tween 20, 10% Glycerol), then with 200 ml Buffer Z (50 mM Tris8, 200 mM NaCl, 0.1% Tween 20, 10% Glycerol) and 200 ml PBS. The estimated concentration of mutant NAD-14 ligase on the beads was 69 μM of ligase per ml of beads.

The following template DNA oligonucleotide and segment oligonucleotides (table 21) were synthesized by standard solid phase methods.

TABLE 21

| | | | % HPLC |
|---|---|---|---|
| Segment | Sequence | MW | purity |
| Template 3 | 5' TTTGGTGCGAAGCAGACTGA | | |
| | GGC-3' | | |
| | (SEQ ID NO: 30) | | |
| centre | 5'-(p)dTdCdTdGdCdT-3' | 1865.4 | 97.5 |
| segment | | | |
| MOE 3'- | 5'-(p)dTdCmGmCmAmCmC-3' | 2546.7 | 98.0 |
| segment | | | |
| MOE 5'- | 5'-mGmCmCmYmCdAdG-3' | 2492.7 | 98.3 |
| segment | | | |

(p) = phosphate, mX = MOE bases, dX = DNA bases
all 5-methyl pyrimidines
all linkages are phosphodiester linkages

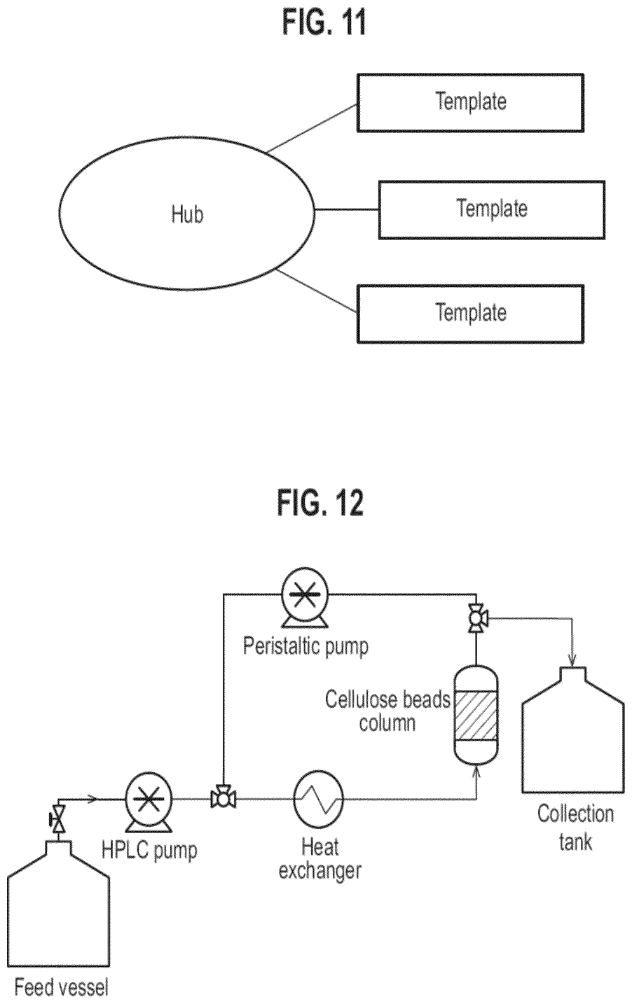

A “tri-template hub” (approximately 24 kDa) comprising a support material referred to as the “hub” and three template sequences, was produced (FIG. 11). Each copy of the template was covalently attached, at its own individual attachment point, to the “hub”. The “tri-template hub” molecule has a higher molecular weight than the target (product) oligonucleotide (100% complementary to the template sequence), thereby allowing it to be retained when the impurities and products are separated from the reaction mixture. It should be noted that in this particular case the template sequence was SEQ ID NO:30 and three copies were attached to the hub. In the following example, a tri-template hub is also produced, but with a different template sequence. Accordingly, as the template sequence varies between examples, so too does the tri-template hub.

The following reaction mix (total volume 5 ml) was prepared:

| | | | |
|---|---|---|---|
| 250 | μl | 1M KH₂PO₄, pH 7.5 | (50 mM final) |
| 108 | μl | 0.07011M centre segment | (1.5 mM final) |
| 137 | μl | 0.05481M 3′-segment | (1.5 mM final) |
| 168 | μl | 0.04461M 5′-segment | (1.5 mM final) |
| 750 | μl | 0.00387M Hub (Template) | (0.55 mM final) |
| 350 | μl | 50 mM NAD⁺ | (3.5 mM final) |
| 1000 | μl | 50 mM MgCl₂ | (10 mM final) |
| 2237 | μl | Nuclease free H₂O | |

### 13.2 Methods

A semi-continuous system was set up as shown in FIG. 12.

4 ml of PERLOZA beads and immobilised mutant NAD-14 ligase were packed into a Pharmacia XK16 column (B). A water bath and peristaltic pump (C) was used to keep the temperature of the column, using the column water compartment, at 30° C. The beads were equilibrated by running 120 ml (30×column volume) of buffer containing 50 mM KH₂PO₄ at pH7.5 for 120 minutes at 1 ml/min. An AKTA explorer pump A1 (A) was used to create the flow through the Pharmacia XK16 column.

Following column equilibration, the 5 ml reaction mix (mixed well by vortexing) was loaded onto the column, collected in the reservoir tube (D) and recirculated through the column using the AKTA explorer A1 pump. The reaction mix was recirculated through the system at a flow rate of 1 ml/min in continuous circulation mode for 16 hours. Samples were collected after 30 minutes, 60 minutes, 90 minutes, 4 hours, 5 hours, 6 hours, 7 hours, 14 hours and 16 hours for HPLC analysis.

### 13.3 Results and Conclusions

TABLE 22

| | | | Centre | | 5′ + centre | |
|---|---|---|---|---|---|---|
| | | 5′-segment | segment | 3′-segment | intermediate | Product |
| Sample | | (%) | (%) | (%) | (%) | (%) |
| 30 | min | 7.80 | 11.00 | 24.00 | 39.40 | 4.40 |
| 60 | min | 3.30 | 4.50 | 18.80 | 41.50 | 18.90 |
| 90 | min | 2.10 | 2.80 | 15.10 | 33.80 | 34.40 |
| 4 | hr | 1.80 | 2.40 | 9.60 | 19.20 | 56.80 |
| 5 | hr | 1.72 | 2.40 | 8.30 | 15.70 | 61.50 |
| 6 | hr | 1.69 | 2.50 | 8.60 | 15.90 | 69.40 |
| 7 | hr | 1.70 | 2.40 | 8.30 | 14.80 | 70.90 |
| 14 | hr | 2.00 | 2.70 | 1.60 | 5.40 | 88.10 |
| 16 | hr | 0.80 | 1.90 | 1.70 | 3.30 | 89.50 |

The percentage of each segment, intermediate and product is expressed as fractional peak area relative to the tri-template hub peak area.

In conclusion, the semi-continuous flow reaction worked and after 16 hours the reaction was almost complete.

Example 14: Separating Oligonucleotides of Different Sizes by Filtration: A) Separation of a 20-Mer Oligonucleotide (SEQ ID NO:1) and a Hub Comprising Three Non-Complementary 20-Mer Oligonucleotides (SEQ ID NO:30); (b) Separation of a 20-Mer Oligonucleotide (SEQ ID NO:1) from Segment 6-Mer and 8-Mer Oligonucleotides (See Table 1) and a Hub Comprising Three Complementary 20-Mer Oligonucleotides (SEQ ID NO:2)

### 14.1 Materials

All oligonucleotides used were synthesized by standard solid phase methods.

A tri-template hub, as described in 13.1 (FIG. 11), was used.

A variety of filters of varying molecular weight cut-offs and from different manufacturers were used as shown in tables 23 and 24.

### 14.2 Methods

### 14.2.1 Dead-End Filtration Set-Up and Protocol for Screening of Polymeric Membranes: (Protocol 1)

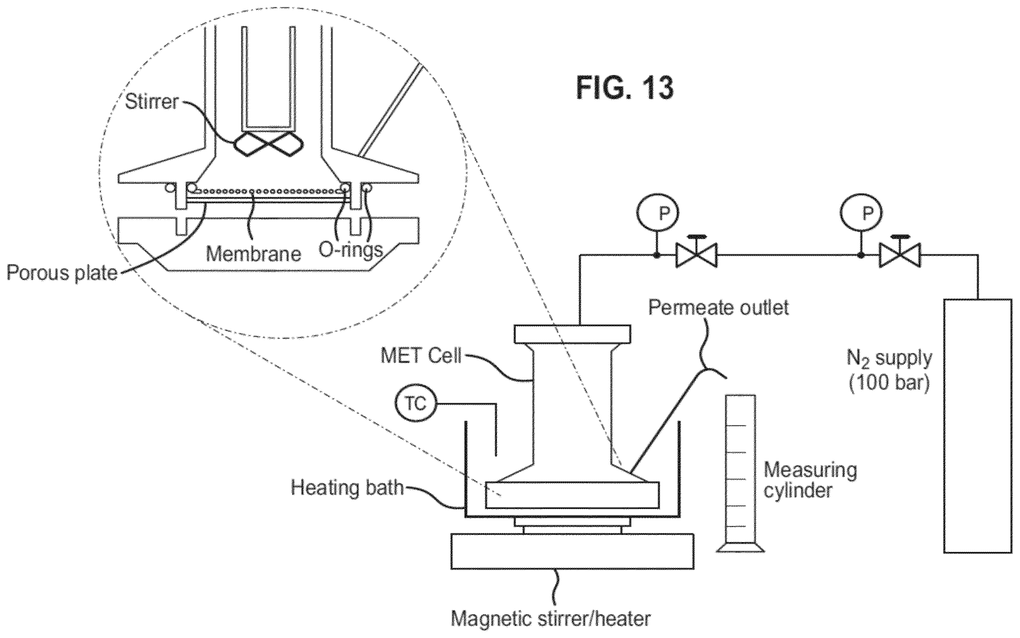

A dead-end filtration rig was set-up as shown in FIG. 13 comprising a MET dead-end filtration cell placed in a water bath sitting on a magnetic stirrer and hotplate. Pressure inside the cell was provided from a flow of nitrogen.

The coupon of membrane (14 cm²) to be tested was first cut to the appropriate size and placed in the cell. The membrane was first conditioned with HPLC grade water (200 ml) and then with PBS buffer (200 ml). The cell was then depressurised, the remaining PBS solution was removed and replaced by a solution containing oligonucleotides (40 ml of oligonucleotides in PBS at a 1 g/L concentration). The cell was placed on a hot stirrer plate and the solution was heated to the desired temperature while being stirred using magnetic agitation. Pressure was applied to the cell (aiming for approximately 3.0 bar; the actual pressure was recorded in each case). Stirring of the solution was either stopped or continued and permeate solution was collected (approximately 20 ml) and analysed by HPLC. Flux was recorded. The system was then depressurised to allow sampling and analysis by HPLC of the retentate solution. More PBS buffer (20 ml) was then added to the filtration cell and the previous procedure was repeated 3 times. The membrane was finally washed with PBS buffer.

All samples were analysed by HPLC without any dilution.

### 14.2.2 Cross-Flow Filtration Set-Up and Protocol for Screening of Polymeric Membranes: (Protocol 2)

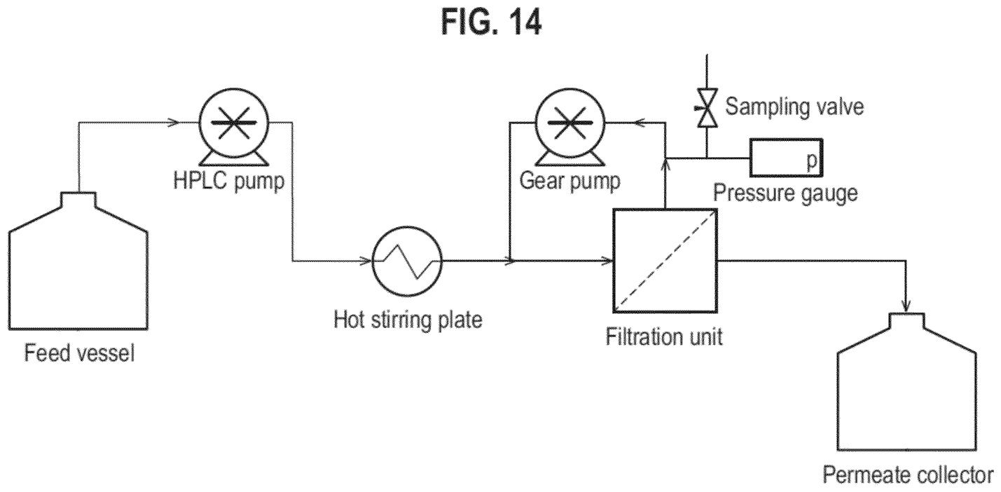

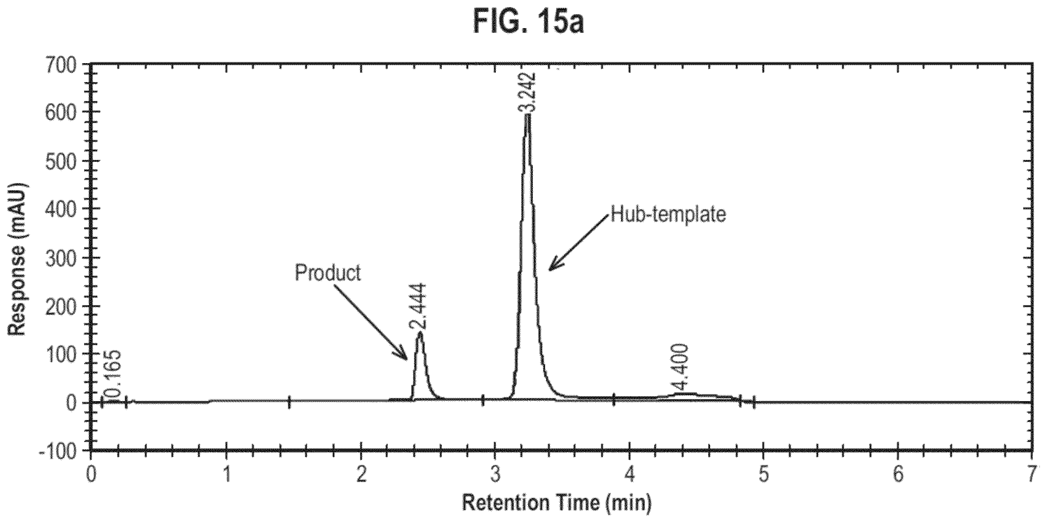

A cross-flow filtration rig was set-up as shown in FIG. 14. The feed vessel (1) consisting of a conical flask contained the oligonucleotide solution to be purified. The solution was pumped to a cross-flow filtration cell (4) using an HPLC pump (2) while temperature within the cell was maintained using a hot plate (3). The solution within the cell was recirculated using a gear pump (6). A pressure gauge (5) enabled the pressure to be read during the experiment. Samples of the retentate solution were taken from the sampling valve (7) while the permeate solution was sampled from the permeate collection vessel (8).

The coupon of membrane to be tested was first cut to the appropriate size and placed in the cell. The system was washed with a PBS solution (100 ml). Temperature of the solution was adjusted to the desired set point. A solution containing oligonucleotide products in PBS (7.5 ml at 1 g/L) was fed into the system. PBS solution was then pumped into the system using the HPLC pump at a flow rate matching the flow rate of the permeate solution (typically 3 ml/min). Pressure was recorded using the pressure gauge. The retentate solution was sampled for HPLC analysis every 5 diafiltration volumes. The permeate solution was sampled for HPLC analysis every diafiltration volume. The experiment was stopped after 20 diafiltration volumes.

In the case of the experiment using the Snyder membrane having a 5 kDa molecular weight cut off (lot number 120915R2) and SEQ ID NO:46 and SEQ ID NO:30 the above methodology was modified as follows. The coupon of membrane to be tested was first cut to the appropriate size and placed in the cell. The system was washed with a Potassium phosphate solution (100 ml, 50 mM, pH 7.5). Temperature of the solution was adjusted to the desired set point. A solution containing oligonucleotide products in potassium phosphate (approximately 1 g/L) was added to ethylenediaminetetraacetic acid (EDTA) (230 μL of a 500 mM solution). The solution was then fed into the system. Potassium phosphate buffer was then pumped into the system using the HPLC pump at a flow rate matching the flow rate of the permeate solution (typically 4 ml/min). Pressure was recorded using the pressure gauge. The retentate solution was sampled for HPLC analysis every 5 diafiltration volumes. The permeate solution was sampled for HPLC analysis every diafiltration volume. The experiment was stopped after 15 diafiltration volumes

### 14.3 Results

TABLE 23
results for the dead-end filtration experiments following protocol 1 (14.2.1)

| | | | | | | Rejection (%) | |
|---|---|---|---|---|---|---|---|
| | | | | | | | Tri- |
| Stirring | | MWCO | Lot | | Temp. | | template |
| (Yes or No) | Membrane | (kDa) | number | SEQ ID NO | (° C.) | Product | hub |
| Yes | NADIR | 10 | 226162 | 1 and 30 | 60 | 38 | 96 |
| Yes | NADIR | 10 | 226162 | 1 and 2 | 60 | 90 | 98 |
| | | | | | 80 | 40 | 43 |
| Yes | NADIR | 10 | 226162 | 1 and 2 | 60 | 83 | 92 |
| | | | | | 65 | 77 | 84 |
| | | | | | 70 | 38 | 41 |
| | | | | | 75 | 12 | 20 |
| Yes | NADIR | 5 | 226825 | 1 and 2 | 60 | 97 | 95 |
| | | | | | 65 | 99 | 93 |
| | | | | | 70 | 97 | 92 |
| | | | | | 75 | 90 | 95 |
| No | NADIR | 5 | 226825 | 1 and 2 | 75 | 66 | 96 |
| No | NADIR | 10 | 226162 | 1 and 2 | 75 | 80 | 80 |
| No | Snyder | 5 | 120915R2 | 1 and 2 | 75 | 97 | 98 |
| No | Osmonics | 5 | 622806PT | 1 and 2 | 75 | 99 | 98 |
| No | Osmonics | 10 | 622806PW | 1 and 2 | 75 | 86 | 95 |

MWCO = molecular weight cut-off

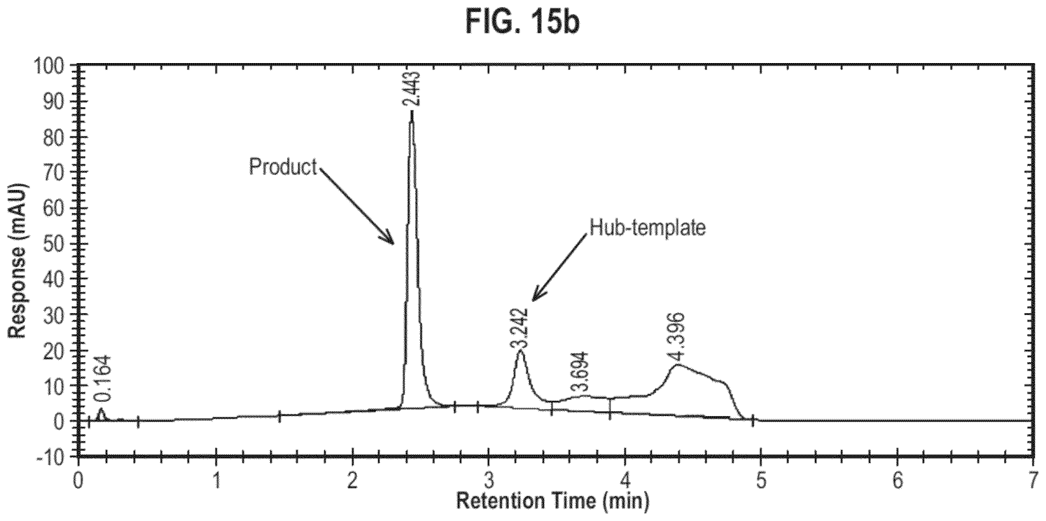

In the experiment using the 10 kDa MWCO NADIR membrane at 60° C., clear separation between the product sequence (SEQ ID NO:1) and the non-complementary tri-template hub (comprising SEQ ID NO:30) was demonstrated. FIG. 15 shows a) a chromatogram of the retentate solution, which remained in the filtration cell and contained mainly tri-template hub, after two diafiltration volumes; and b) a chromatogram of the permeate, solution enriched in the product, after two diafiltration volumes.

TABLE 24
results for the cross-flow filtration experiments (following protocol 2) (14.2.2)

| | | | | | | | Rejection (%) | | | | |
|---|---|---|---|---|---|---|---|---|---|---|---|
| | | | | | | | | | | | Tri- |
| | MWCO | Lot | | Temp. | | Diafiltration | | | | | template |
| Membrane | (kDa) | number | SEQ ID NO | (° C.) | Pressure | volume | Segment 1 | Segment 2 | Segment 3 | Product | hub |
| Osmonics | 10 | 622806PW | 1 and 2 | 75 | 3.1 | 0 | N/A | N/A | N/A | 100 | 88 |
| | | | | | | 5 | N/A | N/A | N/A | 64 | 88 |
| | | | | | | 10 | N/A | N/A | N/A | 40 | 82 |
| | | | | | | 15 | N/A | N/A | N/A | 12 | 74 |
| Snyder | 5 | 120915R2 | 1 and 2 | 75 | 3.1 | 0 | N/A | N/A | N/A | 100 | 100 |
| | | | | 82 | | 5 | N/A | N/A | N/A | 88 | 100 |
| | | | | 75 | | 10 | N/A | N/A | N/A | 98 | 100 |
| | | | | 75 | | 15 | N/A | N/A | N/A | 98 | 100 |
| | | | | 75 | | 20 | N/A | N/A | N/A | 96 | 100 |
| Snyder | 5 | 120915R2 | 1 and 2 | 85 | 3.1 | 0 | N/A | N/A | N/A | 99 | 99 |
| | | | | | | 5 | N/A | N/A | N/A | 63 | 99 |
| | | | | | | 10 | N/A | N/A | N/A | 79 | 99 |
| | | | | | | 15 | N/A | N/A | N/A | 100 | 98 |
| | | | | | | 20 | N/A | N/A | N/A | 100 | 99 |
| Snyder | 5 | 120915R2 | 1 and 2+ | 50 | 3.0 | 0 | 100 | 100 | 100 | 100 | 100 |
| | | | segments from | | | 5 | 39 | 39 | 40 | 100 | 100 |
| | | | table 1 | | | 10 | 82 | 86 | 86 | 100 | 100 |
| | | | | | | 15 | 77 | 81 | 79 | 100 | 100 |
| | | | | | | 20 | * | * | * | 100 | 100 |
| Snyder | 5 | 120915R2 | 1 and 2 | 80 | 3.1 | 0 | N/A | N/A | N/A | 96 | 100 |
| | | | | | | 5 | N/A | N/A | N/A | 22 | 96 |
| | | | | | | 10 | N/A | N/A | N/A | 10 | 100 |
| | | | | | | 20 | N/A | N/A | N/A | 38 | 100 |
| Snyder | 5 | 120915R2 | 46 and 30 | 80 | 3.4 | 0 | N/A | N/A | N/A | 98 | 100 |
| | | | | | | 5 | N/A | N/A | N/A | 83 | 100 |
| | | | | | | 10 | N/A | N/A | N/A | 68 | 100 |
| | | | | | | 15 | N/A | N/A | N/A | 72 | 100 |

MWCO = molecular weight cut-off
* concentration of solutes too low preventing meaningful analysis

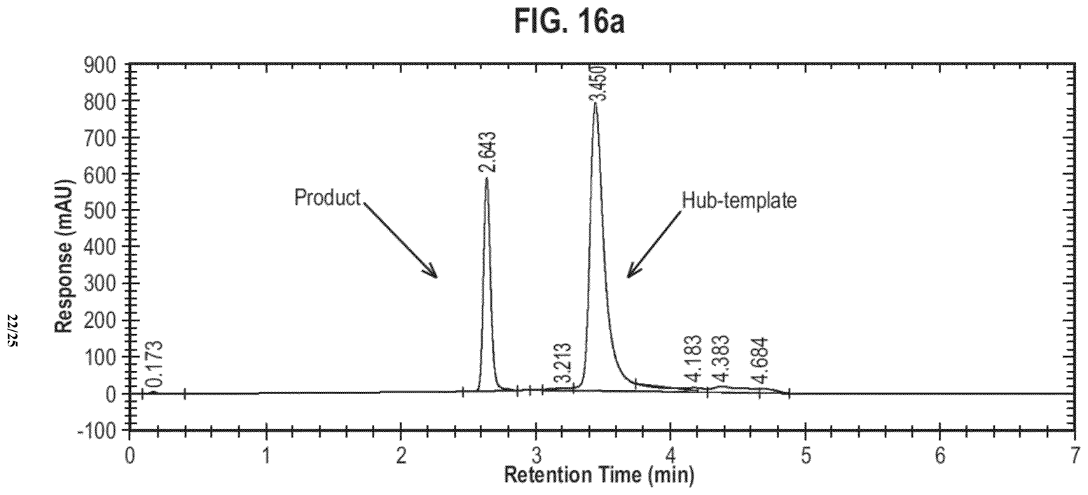

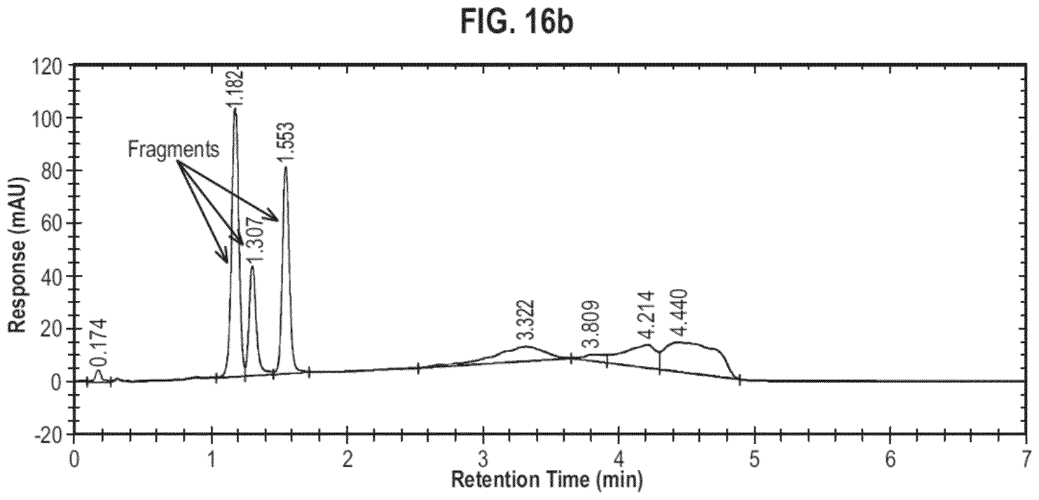

In the experiment using the 5 kDa MWCO Snyder membrane at 50° C. and 3.0 bar pressure, clear separation between the segment sequences (see table 1) and the complementary tri-template hub (comprising SEQ ID NO:2) and product (SEQ ID NO:1) was demonstrated. FIG. 16 shows a) a chromatogram of the retentate solution, which contained mainly tri-template hub and product, after 20 diafiltration volumes; and b) a chromatogram of the permeate, which contained mainly segment oligonucleotides, after 20 diafiltration volumes.

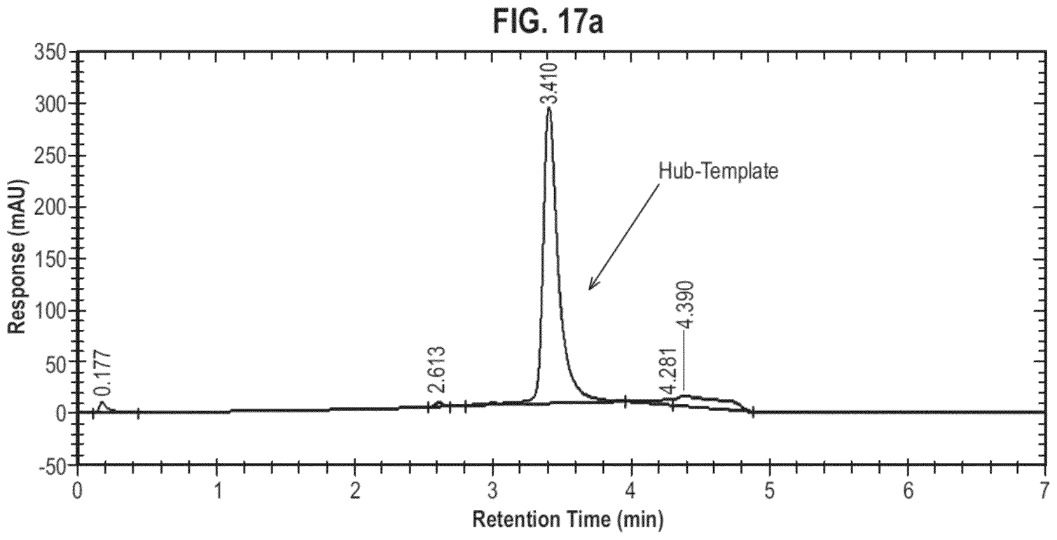

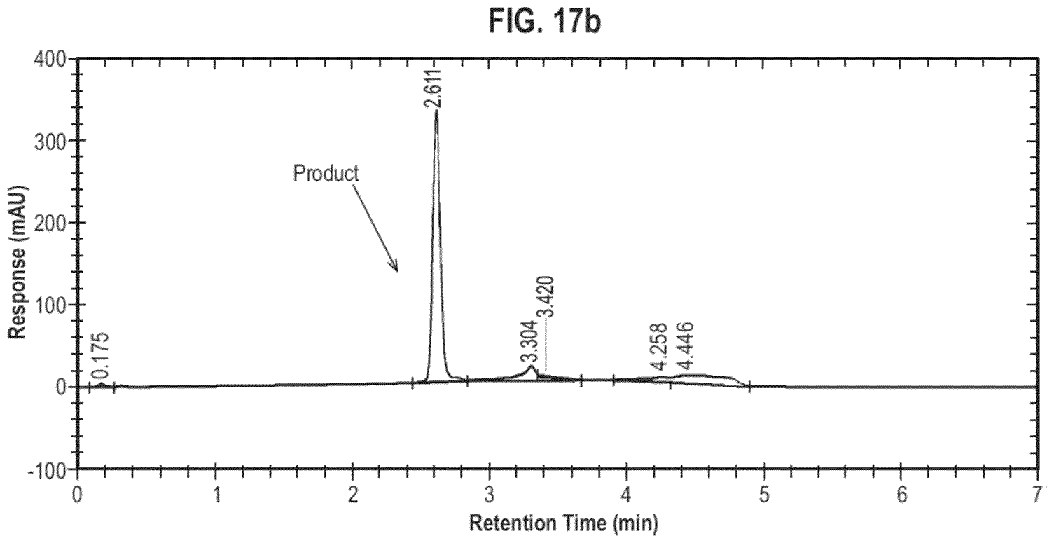

In the experiment using the 5 kDa MWCO Snyder membrane at 80° C. and 3.1 bar pressure, clear separation between the complementary tri-template hub (comprising SEQ ID NO:2) and product (SEQ ID NO:1) was demonstrated. FIG. 17 shows a) a chromatogram of the retentate solution, which contained tri-template hub only, after 20 diafiltration volumes; and b) a chromatogram of the permeate solution, which contained the product only, after 2 diafiltration volumes.

### 14.4 Conclusions

Oligonucleotides of different lengths and molecular weights can be separated using filtration. As shown above, the type of membrane and the conditions, such as temperature, affect the level of separation. For a given set of oligonucleotides of different lengths/molecular weights, suitable membranes and conditions can be selected to allow the required separation. For example, we have demonstrated that segment oligonucleotides (shortmers of 6 and 8 nucleotides in length), as outlined in table 1, can be separated from the product oligonucleotide (20-mer oligonucleotide having SEQ ID NO:1) and tri-template hub (comprising 3×20-mer of SEQ ID NO:2 attached to a solid support), and the product oligonucleotide and tri-template hub can, in turn, be separated from each other.

### Example 15: 3′ Extension by Single Base Ligation for Oligonucleotide Synthesis with 2′-OMe Base Modification and Phosphate Linkages

Wild-type ssLigases (SEQ ID NO:63 to 83) were each fused at the N-terminus to a histidine tag consisting of 6×His. Genes were synthesised, cloned into pET28a and protein encoding the gene produced in E. coli BL21(DE3) Star using standard cloning, expression and extraction methods. Proteins were purified using Ni-NTA using standard methods and used directly.

Reactions were set up as per table 25 below, with addition of ligase last. Reactions were incubated at 25° C. for 20 hours. Reactions were then quenched by heating to 95° C. for 15 min, centrifuged at 4000×g for 15 min and 10 μL of supernatant transferred to an HPLC vial. Reactions were then analysed by HPLC for the presence of SEQ ID NO:85.

TABLE 25

| | | Volume per |
|---|---|---|
| | Final concentration | reaction |
| | in reaction (mM) | (μL) |
| Primer: (2′-OMe) - SEQ ID NO: 84 | 0.125 | 2.5 |
| Monomer: Adenosine-3,5-bisphosphate (2′OMe pAp) | 1 | 2 |
| Adenosine triphosphate trisodium (ATP) | 0.25 | 0.5 |
| Magnesium Chloride (MgCl₂) | 10 | 0.2 |
| Ligase - SEQ ID NO: 63 to 83 | | 5 |
| Buffer (20 mM Tris, 30 mM NaCl at pH 8) | | 9.8 |
| Final reaction volume: | | 20 |

TABLE 26
Results from reactions:

| | SED | Conversion |
|---|---|---|
| | ID | (%) to |
| Gene name | NO | product* |
| Enterobacteria phage T4 RNA ligase 1 - ‘Rnl1’ | 63 | 23.9 |
| Enterobacteria phage T4 RNA ligase 2 - ‘Rnl2’ | 64 | 12.1 |
| Aeromonas virus Aeh1 ligase - ‘RNA3’ | 65 | 15.7 |
| Klebsiella phage JD18 ligase - ‘RNA6’ | 66 | 25.5 |
| Stenotrophomonas phage IME13 ligase - ‘RNA8’ | 67 | 29.9 |
| Escherichia phage JSE ligase - ‘RNA9’ | 68 | 16.7 |
| Acinetobacter phage Acj61 ligase - ‘RNA16’ | 69 | 18.0 |
| Vibrio phage VH7D ligase - ‘RNA20’ | 70 | 14.4 |
| Escherichia phage JS98 ligase - ‘RNA22’ | 71 | 23.9 |
| Escherichia phage vB_EcoM_112 ligase - | 72 | 24.7 |
| ‘RNA23’ | | |
| Aeromonas phage CC2 ligase - ‘RNA28’ | 73 | 19.3 |
| Enterobacteria phage RB69 ligase - ‘RNA29’ | 74 | 19.1 |
| Acinetobacter phage Ac42 ligase - ‘RNA31 | 75 | 24.2 |
| Pectobacterium phage CBB ligase - ‘RNA33’ | 76 | 0.0 |
| Aeromonas phage phiAS5 ligase - ‘RNA35 | 77 | 22.4 |
| Salmonella phage vB_SenMS16 ligase - ‘RNA36 | 78 | 22.0 |
| Vibrio phage KVP40 ligase - ‘RNA39’ | 79 | 20.2 |
| Campylobacter virus CP220 ligase - ‘RNA49’ | 80 | 10.4 |
| Pseudomonas phage phiPMW ligase - ‘RNA59’ | 81 | 2.2 |
| Pseudomonas phage vB_PaeM_C2-10_Ab1 | 82 | 3.4 |
| ligase - ‘RNA61’ | | |
| Butyrivibrio proteoclasticus ligase - ‘RNA82’ | 83 | 2.3 |

*Conversion (%) to product - % area of Primer N peak vs. Product N + 1(pi) peak at wavelength 258 nm

### Example 16: 3′ Extension by Single Base Ligation for Oligonucleotide Synthesis with 2′-OMe Base Modification and Phosphorothioate Linkages

Wild-type ssLigases (SEQ ID NO:63 to 83) were each fused at the N-terminus to a histidine tag consisting of 6×His. Genes were synthesised, cloned into pET28a and protein encoding the gene produced in E. coli BL21(DE3) Star using standard cloning, expression and extraction methods. Proteins were purified using Ni-NTA using standard methods and used directly.

Reactions were set up as per table 27 below, with addition of ligase last. Reactions were incubated at 25° C. for 20 hours. Reactions were then quenched by heating to 95° C. for 15 min, centrifuged at 4000×g for 15 min and 10 μL of supernatant transferred to an HPLC vial. Reactions were then analysed by HPLC for the presence of SEQ ID NO:85.

TABLE 27

| | | Volume |
|---|---|---|
| | Final concentration | per reaction |
| | in reaction (mM) | (L) |
| Primer: (2′-OMe) - SEQ ID NO: 84 | 0.125 | 2.5 |
| Monomer: Adenosine-3-phosphate-5-thiophosphate | 1 | 2 |
| (2′OMe psAp) | | |
| Adenosine triphosphate trisodium (ATP) | 0.25 | 0.5 |
| Magnesium Chloride (MgCl₂) | 10 | 0.2 |
| Ligase - SEQ ID NO: 63 to 83 | | 5 |
| Buffer (20 mM Tris, 30 mM NaCl at pH 8) | | 9.8 |
| Final reaction volume: | | 20 |

TABLE 28
Results from reactions:

| | SED | Conversion |
|---|---|---|
| | ID | (%) to |
| Gene name | NO | product* |
| Enterobacteria phage T4 RNA ligase 1 - ‘Rnl1’ | 63 | 0 |
| Enterobacteria phage T4 RNA ligase 2 - ‘Rnl2′ | 64 | 0 |
| Aeromonas virus Aeh1 ligase - ‘RNA3’ | 65 | 0 |
| Klebsiella phage JD18 ligase - ‘RNA6’ | 66 | 0.8 |
| Stenotrophomonas phage IME13 ligase - ‘RNA8’ | 67 | 2.8 |
| Escherichia phage JSE ligase - ‘RNA9’ | 68 | 1.5 |
| Acinetobacter phage Acj61 ligase - ‘RNA16’ | 69 | 0 |
| Vibrio phage VH7D ligase - ‘RNA20’ | 70 | 0 |
| Escherichia phage JS98 ligase - ‘RNA22′ | 71 | 0 |
| Escherichia phage vB_EcoM_112 ligase - | 72 | 0 |
| ‘RNA23’ | | |
| Aeromonas phage CC2 ligase - ‘RNA28’ | 73 | 0 |
| Enterobacteria phage RB69 ligase - ‘RNA29’ | 74 | 0 |
| Acinetobacter phage Ac42 ligase - ‘RNA31 | 75 | 1.2 |
| Pectobacterium phage CBB ligase - ‘RNA33’ | 76 | 0 |
| Aeromonas phage phiAS5 ligase - ‘RNA35 | 77 | 0 |
| Salmonella phage vB_SenMS16 ligase - ‘RNA36 | 78 | 0 |
| Vibrio phage KVP40 ligase - ‘RNA39’ | 79 | 0 |
| Campylobacter virus CP220 ligase - ‘RNA49’ | 80 | 0 |
| Pseudomonas phage phiPMW ligase - ‘RNA59’ | 81 | 0 |
| Pseudomonas phage vB_PaeM_C2-10_Ab1 | 82 | 0 |
| ligase - ‘RNA61’ | | |
| Butyrivibrio proteoclasticus ligase - ‘RNA82′ | 83 | 0 |

*Conversion (%) to product - % area of Primer N peak vs. Product N + 1(pi) peak at wavelength 258 nm

### Example 17: 3′ Extension by Single Base Ligation for Oligonucleotide Synthesis with 2′MOE Base Modification and Phosphorothioate Linkages

Wild-type ssLigases (SEQ ID NO:63 to 83) were each fused at the N-terminus to a histidine tag consisting of 6×His. Genes were synthesised, cloned into pET28a and protein encoding the gene produced in E. coli BL21(DE3) Star using standard cloning, expression and extraction methods. Proteins were purified using Ni-NTA using standard methods and used directly.

Reactions were set up as per table 29 below, with addition of ligase last. Reactions were incubated at 25° C. for 20 hours. Reactions were then quenched by heating to 95° C. for 15 min, centrifuged at 4000×g for 15 min and 10 μL of supernatant transferred to an HPLC vial. Reactions were then analysed by HPLC for the presence of SEQ ID NO:87.

TABLE 29

| | | Volume |
|---|---|---|
| | Final concentration | per reaction |
| | in reaction (mM) | (μl) |
| Primer: (2-′MOE) - SEQ ID NO: 86 | 0.125 | 2.5 |
| Monomer: Adenosine-3-phosphate-5-thiophosphate | 1 | 2 |
| (2′MOE psAp) | | |
| Adenosine triphosphate trisodium (ATP) | 0.25 | 0.5 |
| Magnesium Chloride (MgCl₂) | 10 | 0.2 |
| Ligase - SEQ ID NO: 63 to 83 | | 5 |
| Buffer (20 mM Tris, 30 mM NaCl at pH 8) | | 9.8 |
| Final reaction volume: | | 20 |

TABLE 30
Results from reactions:

| | SED | Conversion |
|---|---|---|
| | ID | (%) to |
| Gene name | NO | product* |
| Enterobacteria phage T4 RNA ligase 1 - ‘Rnl1’ | 63 | 0 |
| Enterobacteria phage T4 RNA ligase 2 - ‘Rnl2’ | 64 | 0 |
| Aeromonas virus Aeh1 ligase - ‘RNA3’ | 65 | 0 |
| Klebsiella phage JD18 ligase - ‘RNA6’ | 66 | 0 |
| Stenotrophomonas phage IME13 ligase - ‘RNA8’ | 67 | 0.4 |
| Escherichia phage JSE ligase - ‘RNA9’ | 68 | 0 |
| Acinetobacter phage Acj61 ligase - ‘RNA16’ | 69 | 0 |
| Vibrio phage VH7D ligase - ‘RNA20’ | 70 | 0 |
| Escherichia phage JS98 ligase - ‘RNA22’ | 71 | 0 |
| Escherichia phage vB_EcoM_112 ligase - | 72 | 0 |
| ‘RNA23’ | | |
| Aeromonas phage CC2 ligase - ‘RNA28’ | 73 | 0 |
| Enterobacteria phage RB69 ligase - ‘RNA29’ | 74 | 0 |
| Acinetobacter phage Ac42 ligase - ‘RNA31 | 75 | 0.2 |
| Pectobacterium phage CBB ligase - ‘RNA33’ | 76 | 0 |
| Aeromonas phage phiAS5 ligase - ‘RNA35 | 77 | 0 |
| Salmonella phage vB_SenMS16 ligase - ‘RNA36 | 78 | 0 |
| Vibrio phage KVP40 ligase - ‘RNA39’ | 79 | 0 |
| Campylobacter virus CP220 ligase - ‘RNA49’ | 80 | 0 |
| Pseudomonas phage phiPMW ligase - ‘RNA59’ | 81 | 0 |
| Pseudomonas phage vB_PaeM_C2-10_Ab1 ligase | 82 | 0.5 |
| ‘RNA61’ | | |
| Butyrivibrio proteoclasticus ligase - ‘RNA82’ | 83 | 0 |

*Conversion (%) to product - % area of Primer N peak vs. Product N + 1(pi) peak at wavelength 258 nm

### Example 18: 3′ Extension by Single Base Ligation for Oligonucleotide Synthesis with 2′H Base Modification and Phosphate Linkages

Wild-type ssLigases (SEQ ID NO:63 to 83) were each fused at the N-terminus to a histidine tag consisting of 6×His. Genes were synthesised, cloned into pET28a and protein encoding the gene produced in E. coli BL21(DE3) Star using standard cloning, expression and extraction methods. Proteins were purified using Ni-NTA using standard methods and used directly.

Reactions were set up as per table 31 below, with addition of ligase last. Reactions were incubated at 25° C. for 20 hours. Reactions were then quenched by heating to 95° C. for 15 min, centrifuged at 4000×g for 15 min and 10 μL of supernatant transferred to an HPLC vial. Reactions were then analysed by HPLC for the presence of SEQ ID NO:87.

TABLE 31

| | Final | Volume |
|---|---|---|
| | concentration | per |
| | in reaction | reaction |
| | (mM) | (μL) |
| Primer: (2′H) - SEQ ID NO: 86 | 0.125 | 2.5 |
| Monomer: Adenosine-3-phosphate-5- | 1 | 2 |
| thiophosphate (2′MOE psAp) | | |
| Adenosine triphosphate trisodium (ATP) | 0.25 | 0.5 |
| Magnesium Chloride (MgCl₂) | 10 | 0.2 |
| Ligase - SEQ ID NO: 63 to 83 | | 5 |
| Buffer (20 mM Tris, 30 mM NaCl at pH 8) | | 9.8 |
| Final reaction volume: | | 20 |

TABLE 32
Results from reactions:

| | SED | Conversion |
|---|---|---|
| | ID | (%) to |
| Gene name | NO | product* |
| Enterobacteria phage T4 RNA ligase 1 - ‘Rnl1’ | 63 | 6.3 |
| Enterobacteria phage T4 RNA ligase 2 - ‘Rnl2’ | 64 | 0 |
| Aeromonas virus Aeh1 ligase - ‘RNA3’ | 65 | 0 |
| Klebsiella phage JD18 ligase - ‘RNA6’ | 66 | 2.3 |
| Stenotrophomonas phage IME13 ligase - ‘RNA8’ | 67 | 5.1 |
| Escherichia phage JSE ligase - ‘RNA9’ | 68 | 1.1 |
| Acinetobacter phage Acj61 ligase - ‘RNA16’ | 69 | 0 |
| Vibrio phage VH7D ligase - ‘RNA20’ | 70 | 0 |
| Escherichia phage JS98 ligase - ‘RNA22’ | 71 | 0 |
| Escherichia phage vB_EcoM_112 ligase - | 72 | 0 |
| ‘RNA23’ | | |
| Aeromonas phage CC2 ligase - ‘RNA28’ | 73 | 2.3 |
| Enterobacteria phage RB69 ligase - ‘RNA29’ | 74 | 0 |
| Acinetobacter phage Ac42 ligase - ‘RNA31 | 75 | 0 |
| Pectobacterium phage CBB ligase - ‘RNA33’ | 76 | 2.2 |
| Aeromonas phage phiAS5 ligase - ‘RNA35 | 77 | 0 |
| Salmonella phage vB_SenMS16 ligase - ‘RNA36 | 78 | 0 |
| Vibrio phage KVP40 ligase - ‘RNA39’ | 79 | 0 |
| Campylobacter virus CP220 ligase - ‘RNA49’ | 80 | 0 |
| Pseudomonas phage phiPMW ligase - ‘RNA59’ | 81 | 0 |
| Pseudomonas phage vB_PaeM_C2-10_Ab1 | 82 | 1.3 |
| ligase - ‘RNA61’ | | |
| Butyrivibrio proteoclasticus ligase - ‘RNA82’ | 83 | 0 |

*Conversion (%) to product - % area of Primer N peak vs. Product N + 1(pi) peak at wavelength 258 nm

### Example 19: 3′ Extension by Single Base Ligation for Oligonucleotide Synthesis with 2′MOE Base Modification and Phosphate Linkages

Wild-type ssLigases (SEQ ID NO:63 to 83) were each fused at the N-terminus to 6×His. Genes were synthesised, cloned into pET28a and protein encoding the gene produced in E. coli BL21(DE3) Star using standard cloning, expression and extraction methods. Proteins were purified using Ni-NTA using standard methods and used directly.

Reactions were set up as per table 33, with addition of ligase last. Reactions were incubated at 25° C. for 20 hours. Reactions were then quenched by heating to 95° C. for 15 min, centrifuged at 4000×g for 15 min and 10 μL of supernatant transferred to an HPLC vial. Reactions were then analysed by HPLC for the presence of SEQ ID NO:87.

TABLE 33

| | Final | |
|---|---|---|
| | concen- | Volume |
| | tration in | per |
| | reaction | reaction |
| | (mM) | (μL) |
| Primer: (2′MOE) - SEQ ID NO: 86 | 0.125 | 2.5 |
| Monomer: Adenosine-3,5-bisphosphate | 1 | 2 |
| (2′MOE pAp) | | |
| Adenosine triphosphate trisodium (ATP) | 0.25 | 0.5 |
| Magnesium Chloride (MgCl₂) | 10 | 0.2 |
| Ligase - SEQ ID NO: 63 to 83 | | 5 |
| Buffer (20 mM Tris, 30 mM NaCl at pH 8) | | 9.8 |
| Final reaction volume: | | 20 |

TABLE 34
Results from reactions:

| | SEQ | Conversion |
|---|---|---|
| | ID | (%) to |
| Gene name | NO | product* |
| Enterobacteria phage T4 RNA ligase 1 - ‘Rnl1’ | 63 | 1.4 |
| Enterobacteria phage T4 RNA ligase 2 - ‘Rnl2’ | 64 | 0 |
| Aeromonas virus Aeh1 ligase - ‘RNA3’ | 65 | 0 |
| Klebsiella phage JD18 ligase - ‘RNA6’ | 66 | 3.9 |
| Stenotrophomonas phage IME13 ligase - ‘RNA8’ | 67 | 8.8 |
| Escherichia phage JSE ligase - ‘RNA9’ | 68 | 0 |
| Acinetobacter phage Acj61 ligase - ‘RNA16’ | 69 | 0 |
| Vibrio phage VH7D ligase - ‘RNA20’ | 70 | 0 |
| Escherichia phage JS98 ligase - ‘RNA22’ | 71 | 0 |
| Escherichia phage vB_EcoM_112 ligase - | 72 | 1.2 |
| ‘RNA23’ | | |
| Aeromonas phage CC2 ligase - ‘RNA28’ | 73 | 0 |
| Enterobacteria phage RB69 ligase - ‘RNA29’ | 74 | 1.1 |
| Acinetobacter phage Ac42 ligase - ‘RNA31 | 75 | 4.0 |
| Pectobacterium phage CBB ligase - ‘RNA33’ | 76 | 0 |
| Aeromonas phage phiAS5 ligase - ‘RNA35 | 77 | 0 |
| Salmonella phage vB_SenMS16 ligase - ‘RNA36 | 78 | 0 |
| Vibrio phage KVP40 ligase - ‘RNA39’ | 79 | 0 |
| Campylobacter virus CP220 ligase - ‘RNA49’ | 80 | 0 |
| Pseudomonas phage phiPMW ligase - ‘RNA59’ | 81 | 0 |
| Pseudomonas phage vB_PaeM_C2-10_Ab1 | 82 | 1.3 |
| ligase - ‘RNA61’ | | |
| Butyrivibrio proteoclasticus ligase - ‘RNA82’ | 83 | 0 |

*Conversion (%) to product - % area of Primer N peak vs. Product N + 1(pi) peak at wavelength 258 nm

### Example 20: Optimisation of 3′ Extension by Single Base Ligation for Oligonucleotide Synthesis with 2′OMe Base Modification and Phosphate Linkages

Wild-type ssLigase (SEQ ID NO:67) was fused at the N-terminus to 6×His. Gene was synthesised, cloned into pET28a and protein encoding the gene produced in E. coli BL21(DE3) Star using standard cloning, expression and extraction methods. Protein was purified using Ni-NTA using standard methods and used directly. Protein concentration was determined via microfluidic capillary electrophoresis.

Reactions were set up as per table 35, with addition of ligase last. Reactions were incubated at 25° C. for 18 hours. Reactions were then quenched by heating to 95° C. for 15 min, centrifuged at 4000×g for 15 min and 10 μL of supernatant transferred to an HPLC vial. Reactions were then analysed by HPLC for the presence of SEQ ID NO:85.

TABLE 35

| | Final | |
|---|---|---|
| | concen- | Volume |
| | tration in | per |
| | reaction | reaction |
| | (mM) | (μL) |
| Primer: (2′OMe) - SEQ ID NO: 84 | 0.125 | 2.5 |
| Monomer: Adenosine-3,5-bisphosphate | 1 | 2 |
| (2′OMe pAp) | | |
| Adenosine triphosphate trisodium (ATP) | 0.25 | 0.5 |
| Magnesium Chloride (MgCl₂) | 10 | 0.2 |
| Ligase - SEQ ID NO: 67 | 0.025 | 10 |
| Buffer (20 mM Tris, 30 mM NaCl at pH 8) | | 4.8 |
| Final reaction volume: | | 20 |

TABLE 36
Results from reaction:

| | SEQ | |
|---|---|---|
| | ID | Conversion |
| Gene name | NO | (%) to product* |
| Stenotrophomonas phage IME13 ligase - ‘RNA8’ | 67 | 66.9 |

*Conversion (%) to product - % area of Primer N peak vs. Product N + 1(pi) peak at wavelength 258 nm

### Example 21: Optimisation of 3′ Extension by Single Base Ligation for Oligonucleotide Synthesis with 2′MOE Base Modification and Phosphate Linkages

Wild-type ssLigase (SEQ ID NO:67) was fused at the N-terminus to 6×His. Gene was synthesised, cloned into pET28a and protein encoding the gene produced in E. coli BL21(DE3) Star using standard cloning, expression and extraction methods. Protein was purified using Ni-NTA using standard methods and used directly. Protein concentration was determined via microfluidic capillary electrophoresis.

Reactions were set up as per table 37, with addition of ligase last. Reactions were incubated at 25° C. for 12 hours. Reactions were then quenched by heating to 95° C. for 15 min, centrifuged at 4000×g for 15 min and 10 μL of supernatant transferred to an HPLC vial. Reactions were then analysed by HPLC for the presence of SEQ ID NO:87.

TABLE 37

| | Final | |
|---|---|---|
| | concen- | Volume |
| | tration in | per |
| | reaction | reaction |
| | (mM) | (μL) |
| Primer: (2′MOE) - SEQ ID NO: 86 | 0.125 | 2.5 |
| Monomer: Adenosine-3,5- | 1 | 2 |
| bisphosphate (2′MOE pAp) | | |
| Adenosine triphosphate trisodium (ATP) | 0.25 | 0.5 |
| Magnesium Chloride (MgCl₂) | 10 | 0.2 |
| Ligase - SEQ ID NO: 67 | 0.025 | 10 |
| Buffer (20 mM Tris, 30 mM NaCl at pH 8) | | 4.8 |
| Final reaction volume: | | 20 |

TABLE 38
Results from reaction:

| | SEQ | Conversion |
|---|---|---|
| | ID | (%) to |
| Gene name | NO | product* |
| Stenotrophomonas phage IME13 ligase - ‘RNA8’ | 67 | 26.0 |

*Conversion (%) to product - % area of SEQ ID NO: 86 peak vs. SEQ ID NO: 87 peak at wavelength 258 nm

### Example 22: Reactions Comparing WT to Mutants-3′ Extension by Single Base Ligation for Oligonucleotide Synthesis with 2′MOE Base Modification and Phosphate Linkages

Wild-type or Mutant ssLigases (SEQ ID NO:67 and 88 to 92) were each fused at the N-terminus to 6×His. Genes were synthesised, cloned into pET28a and protein encoding the gene produced in E. coli BL21(DE3) Star using standard cloning, expression and extraction methods. Proteins were purified using Ni-NTA using standard methods and used directly. Protein concentration was determined via microfluidic capillary electrophoresis.

Reactions were set up as per table 39, with addition of ligase last. Reactions were incubated at 25° C. for 24 hours. Reactions were then quenched by heating to 70° C. for 30 min, centrifuged at 4000×g for 15 min and 10 μL of supernatant transferred to an HPLC vial. Reactions were then analysed by HPLC for the presence of SEQ ID NO:87.

TABLE 39

| | Final | |
|---|---|---|
| | concen- | Volume |
| | tration in | per |
| | reaction | reaction |
| | (mM) | (μL) |
| Primer: (2′MOE) - SEQ ID NO: 86 | 0.1 | 3 |
| Monomer: Adenosine-3,5-bisphosphate | 1 | 3 |
| (2′MOE pAp) | | |
| Adenosine triphosphate trisodium (ATP) | 0.25 | 0.68 |
| Magnesium Chloride (MgCl₂) | 10 | 0.3 |
| Ligase - SEQ ID NO: 67 or 88 to 92 | 0.01 | 7.5 |
| Buffer (20 mM Tris at pH 8) | | 15.53 |
| Final reaction volume: | | 30 |

TABLE 40
Results from reactions:

| | SEQ | Conversion |
|---|---|---|
| | ID | (%) to |
| Gene name | NO | product* |
| Stenotrophomonas phage IME13 ligase - ‘RNA8’ | 67 | 18.34 |
| Mutant ligase (Stenotrophomonas phage IME13 | 88 | 74.18 |
| backbone) protein sequence | | |
| Mutant ligase (Stenotrophomonas phage IME13 | 89 | 72.34 |
| backbone) protein sequence | | |
| Mutant ligase (Stenotrophomonas phage IME13 | 90 | 75.75 |
| backbone) protein sequence | | |
| Mutant ligase (Stenotrophomonas phage IME13 | 91 | 73.99 |
| backbone) protein sequence | | |
| Mutant ligase (Stenotrophomonas phage IME13 | 92 | 55.05 |
| backbone) protein sequence | | |

*Conversion (%) to product - % area of SEQ ID NO: 86 vs. SEQ ID NO: 87 peak at wavelength 258 nm

### Example 23: Reactions Comparing WT to Mutants-3′ Extension by Single Base Ligation for Oligonucleotide Synthesis with 2′MOE Base Modification and Phosphorothioate Linkages

Wild-type or Mutant ssLigases (SEQ ID NO:67 and 88 to 92) were each fused at the N-terminus to 6×His. Genes were synthesised, cloned into pET28a and protein encoding the gene produced in E. coli BL21(DE3) Star using standard cloning, expression and extraction methods. Proteins were purified using Ni-NTA using standard methods and used directly. Protein concentration was determined via microfluidic capillary electrophoresis.

Reactions were set up as per table 41, with addition of ligase last. Reactions were incubated at 25° C. for 24 hours. Reactions were then quenched by heating to 70° C. for 30 min, centrifuged at 4000×g for 15 min and 10 μL of supernatant transferred to an HPLC vial. Reactions were then analysed by HPLC for the presence of SEQ ID NO:87.

TABLE 41

| | Final | |
|---|---|---|
| | concen- | Volume |
| | tration in | per |
| | reaction | reaction |
| | (mM) | (μL) |
| Primer: (2′MOE) - SEQ ID NO: 86 | 0.1 | 3 |
| Monomer: Adenosine-3-phosphate-5- | 1 | 3 |
| thiophosphate (2′OMe psAp) | | |
| Adenosine triphosphate trisodium (ATP) | 0.25 | 0.68 |
| Magnesium Chloride (MgCl₂) | 10 | 0.3 |
| Ligase - SEQ ID NO: 67 or 88 to 92 | 0.01 | 7.5 |
| Buffer (20 mM Tris at pH 8) | | 15.53 |
| Final reaction volume: | | 30 |

TABLE 42
Results from reactions:

| | SEQ | |
|---|---|---|
| | ID | Conversion (%) |
| Gene name | NO | to product* |
| Stenotrophomonas phage IME13 ligase - ‘RNA8’ | 67 | 0 |
| Mutant ligase (Stenotrophomonas phage IME13 | 88 | 19.81 |
| backbone) protein sequence | | |
| Mutant ligase (Stenotrophomonas phage IME13 | 89 | 6.05 |
| backbone) protein sequence | | |
| Mutant ligase (Stenotrophomonas phage IME13 | 90 | 12.56 |
| backbone) protein sequence | | |
| Mutant ligase (Stenotrophomonas phage IME13 | 91 | 12.44 |
| backbone) protein sequence | | |
| Mutant ligase (Stenotrophomonas phage IME13 | 92 | 0 |
| backbone) ligase protein sequence | | |

*Conversion (%) to product - % area of SEQ ID NO: 86 vs. SEQ ID NO: 87 peak at wavelength 258 nm

### Example 24: 3′ Extension by Single Base Ligation for Oligonucleotide Synthesis with 2′MOE Base Modification and Phosphorothioate Linkages-Adenosine

Mutant ssLigase (SEQ ID NO:88) was fused at the N-terminus to 6×His. Gene was synthesised, cloned into pET28a and protein encoding the gene produced in E. coli BL21(DE3) Star using standard cloning, expression and extraction methods. Proteins were purified using Ni-NTA using standard methods and used directly. Protein concentration was determined via microfluidic capillary electrophoresis.

Reactions were set up as per table 43, with addition of ligase last. Reactions were incubated at 25° C. for 24 hours. Reactions were then quenched by heating to 70° C. for 30 min, centrifuged at 4000×g for 15 min and 10 μL of supernatant transferred to an HPLC vial. Reactions were then analysed by HPLC and LCMS for the presence of SEQ ID NO:87.

TABLE 43

| | Final | |
|---|---|---|
| | concen- | Volume |
| | tration in | per |
| | reaction | reaction |
| | (mM) | (μL) |
| Primer: (2′MOE) - SEQ ID NO: 86 | 0.05 | 10 |
| Monomer: Adenosine-3-phosphate-5-thiophosphate | 0.5 | 10 |
| (2′OMe psAp) | | |
| Adenosine triphosphate trisodium (ATP) | 2 | 40 |
| Magnesium Chloride (MgCl₂) | 10 | 2 |
| Ligase - SEQ ID NO: 88 | 0.01 | 21.7 |
| Buffer (20 mM Tris at pH 8) | | 116.3 |
| Final reaction volume: | | 200 |

TABLE 44
Results from reaction:

| | SEQ | Conversion (%) |
|---|---|---|
| Gene name | ID NO | to product* |
| Mutant ligase (Stenotrophomonas phage IME13 | 88 | 94.39 |
| backbone) protein sequence | | |

*Conversion (%) to product - % area of SEQ ID NO: 86 peak vs. SEQ ID NO: 87 peak at wavelength 258 nm

### Example 25: 3′ Extension by Single Base Ligation for Oligonucleotide Synthesis with 2′MOE Base Modification and Phosphorothioate Linkages-5-Methylcytidine

Mutant ssLigase (SEQ ID NO:88) was fused at the N-terminus to 6×His. Gene was synthesised, cloned into pET28a and protein encoding the gene produced in E. coli BL21(DE3) Star using standard cloning, expression and extraction methods. Protein was purified using Ni-NTA using standard methods and used directly. Protein concentration was determined via microfluidic capillary electrophoresis.

Reactions were set up as per table 45, with addition of ligase last. Reactions were incubated at 25° C. for 24 hours. Reactions were then quenched by heating to 70° C. for 30 min, centrifuged at 4000×g for 15 min and 10 μL of supernatant transferred to an HPLC vial. Reactions were then analysed by HPLC and LCMS for the presence of SEQ ID NO:93.

TABLE 45

| | Final | |
|---|---|---|
| | concentration | |
| | in reaction | Volume per |
| | (mM) | reaction (μL) |
| Primer: (2′MOE) - SEQ ID NO: 86 | 0.05 | 10 |
| Monomer: 5-Methylcytidine-3- | 0.5 | 10 |
| phosphate-5-thiophosphate | | |
| (2′OMe psCp) | | |
| Adenosine triphosphate | 2 | 40 |
| trisodium (ATP) | | |
| Magnesium Chloride (MgCl₂) | 10 | 2 |
| Ligase - SEQ ID NO: 88 | 0.01 | 21.7 |
| Buffer (20 mM Tris at pH 8) | | 116.3 |
| Final reaction volume: | | 200 |

TABLE 46
Results from reaction:

| | | Conversion |
|---|---|---|
| Gene name | SEQ ID NO | (%) to product* |
| Mutant ligase (Stenotrophomonas phage | 88 | 69.27 |
| IME13 backbone) protein sequence | | |

*Conversion (%) to product - % area of SEQ ID NO: 86 peak vs. SEQ ID NO: 93 peak at wavelength 258 nm

### Example 26: 3′ Extension by Single Base Ligation for Oligonucleotide Synthesis with 2′MOE Base Modification and Phosphorothioate Linkages-Thymidine

Mutant ssLigase (SEQ ID NO:88) was fused at the N-terminus to 6×His. Gene was synthesised, cloned into pET28a and protein encoding the gene produced in E. coli BL21(DE3) Star using standard cloning, expression and extraction methods. Protein was purified using Ni-NTA using standard methods and used directly. Protein concentration was determined via microfluidic capillary electrophoresis.

Reactions were set up as per table 47, with addition of ligase last. Reactions were incubated at 25° C. for 24 hours. Reactions were then quenched by heating to 70° C. for 30 min, centrifuged at 4000×g for 15 min and 10 μL of supernatant transferred to an HPLC vial. Reactions were then analysed by HPLC and LCMS for the presence of SEQ ID NO:94.

TABLE 47

| | Final | |
|---|---|---|
| | concentration | |
| | in reaction | Volume per |
| | (mM) | reaction (μL) |
| Primer: (2′MOE) - SEQ ID NO: 86 | 0.05 | 10 |
| Monomer: Thymidine-3-phosphate- | 0.5 | 10 |
| 5-thiophosphate (2′OMe psCp) | | |
| Adenosine triphosphate | 2 | 40 |
| trisodium (ATP) | | |
| Magnesium Chloride (MgCl₂) | 10 | 2 |
| Ligase - SEQ ID NO: 88 | 0.01 | 21.7 |
| Buffer (20 mM Tris at pH 8) | | 116.3 |
| Final reaction volume: | | 200 |

TABLE 48
Results from reaction:

| | | Conversion (%) |
|---|---|---|
| Gene name | SEQ ID NO | to product* |
| Mutant ligase (Stenotrophomonas phage | 88 | 94.38 |
| IME13 backbone) protein sequence | | |

*Conversion (%) to product - % area of SEQ ID NO: 86 peak vs. SEQ ID NO: 94 peak at wavelength 258 nm

Example 27: 3′ Extension by Single Base Ligation for Oligonucleotide Synthesis with 2′MOE Base Modification and Phosphorothioate Linkages, Followed by 3′ Dephosphorylation and a Second Single Base Ligation and Second Dephosphorylation-Reaction Sequence

Mutant ssLigase (SEQ ID NO:88) and wild-type phosphatase (SEQ ID NO:95) were fused at the N-terminus to 6×His. Genes were synthesised, cloned into pET28a and protein encoding the gene produced in E. coli BL21(DE3) Star using standard cloning, expression and extraction methods. Proteins were purified using Ni-NTA using standard methods and used directly. Protein concentration was determined via microfluidic capillary electrophoresis.

First ligation reaction was set up as per table 49, with addition of ligase last. Reactions were incubated at 25° C. for 24 hours. Reactions were then quenched by heating to 70° C. for 30 min, centrifuged at 4000×g for 15 min and 10 μL of supernatant transferred to an HPLC vial. Reactions were then analysed by HPLC and LCMS for the presence of SEQ ID NO:87. The remainder of the reactions were purified by ion-exchange chromatography, lyophilised and used directly in first deprotection (i.e. dephosphorylation).

TABLE 49

| | Final | |
|---|---|---|
| | concentration | |
| | in reaction | Volume per |
| | (mM) | reaction (μL) |
| Primer: (2′MOE) - SEQ ID NO: 86 | 0.05 | 10 |
| Monomer: Adenosine-3-phosphate- | 0.5 | 10 |
| 5-thiophosphate (2′MOE psAp) | | |
| Adenosine triphosphate | 2 | 40 |
| trisodium (ATP) | | |
| Magnesium Chloride (MgCl₂) | 10 | 2 |
| Ligase - SEQ ID NO: 88 | 0.01 | 21.7 |
| Buffer (20 mM Tris at pH 8) | | 116.3 |
| Final reaction volume: | | 200 |

TABLE 50
Results from first ligation reaction:

| | | Conversion (%) |
|---|---|---|
| Gene name | SEQ ID NO | to product* |
| Mutant ligase (Stenotrophomonas phage | 88 | 94.99 |
| IME13 backbone) protein sequence | | |

*Conversion (%) to product - % area of SEQ ID NO: 86 peak vs. SEQ ID NO: 87 peak at wavelength 258 nm

First deprotection/dephosphorylation reaction was set up as per table 51, with addition of phosphatase last. Reactions were incubated at 37° C. for 24 hours. Reactions were then quenched by heating to 70° C. for 30 min, centrifuged at 4000×g for 15 min and 10 μL of supernatant transferred to an HPLC vial. Reactions were then analysed by HPLC and LCMS for the presence of SEQ ID NO:96. The remainder of the reactions were purified by ion-exchange chromatography, lyophilised and used directly in the second the ligation reaction.

TABLE 51

| | Final | |
|---|---|---|
| | concentration | Volume per |
| | in reaction (mM) | reaction (μL) |
| First ligation product: (2′MOE) - | 0.05 | 10 |
| SEQ ID NO: 84 | | |
| Phosphatase - SEQ ID NO 95 | 0.00036 | 20 |
| Buffer (20 mM Tris at pH 8) | | 170 |
| Final reaction volume: | | 200 |

TABLE 52
Results from first dephosphorylation reaction:

| | | Conversion |
|---|---|---|
| | | (%) to |
| Gene name | SEQ ID NO | product* |
| Wild type phosphatase | 95 | 96.44 |
| (Pandalus borealis) | | |
| protein sequence - ‘SAP’ | | |

*Conversion (%) to product - % area of SEQ ID NO: 85 peak vs. SEQ ID NO: 96 peak at wavelength 258 nm

Second ligation reaction was set up as per table 53, with addition of ligase last. Reactions were incubated at 25° C. for 24 hours. Reactions were then quenched by heating to 70° C. for 30 min, centrifuged at 4000×g for 15 min and 10 μL of supernatant transferred to an HPLC vial. Reactions were then analysed by HPLC and LCMS for the presence of SEQ ID NO:96. The remainder of the reactions were purified by ion-exchange chromatography, lyophilised and used directly in the second deprotection/dephosphorylation reaction.

TABLE 53

| | Final | |
|---|---|---|
| | concentration | |
| | in reaction | Volume per |
| | (mM) | reaction (μL) |
| First deprotection product: | 0.05 | 10 |
| (2′MOE) - SEQ ID NO: 96 | | |
| Monomer: Adenosine-3-phosphate- | 0.5 | 10 |
| 5-thiophosphate (2′MOE psAp) | | |
| Adenosine triphosphate trisodium (ATP) | 2 | 40 |
| Magnesium Chloride (MgCl₂) | 10 | 2 |
| Ligase - SEQ ID NO: 88 | 0.01 | 21.7 |
| Buffer (20 mM Tris at pH 8) | | 116.3 |
| Final reaction volume: | | 200 |

TABLE 54
Results from second ligation reaction:

| | | Conversion (%) |
|---|---|---|
| Gene name | SEQ ID NO | to product* |
| Mutant ligase (Stenotrophomonas phage | 88 | 91.15 |
| IME13 backbone) protein sequence | | |

*Conversion (%) to product - % area of SEQ ID NO: 97 peak vs. SEQ ID NO: 96 peak at wavelength 258 nm

Second deprotection/dephosphorylation reaction was set up as per table 55, with addition of phosphatase last. Reactions were incubated at 37° C. for 24 hours. Reactions were then quenched by heating to 70° C. for 30 min, centrifuged at 4000×g for 15 min and 10 μL of supernatant transferred to an HPLC vial. Reactions were then analysed by HPLC and LCMS for the presence of SEQ ID NO:98.

TABLE 55

| | Final | |
|---|---|---|
| | concentration | |
| | in reaction | Volume per |
| | (mM) | reaction (μL) |
| Second ligation product: | 0.05 | 10 |
| (2′MOE) - SEQ ID NO: 97 | | |
| Phosphatase - SEQ ID NO: 94 | 0.00036 | 20 |
| Buffer (20 mM Tris at pH 8) | | 170 |
| Final reaction volume: | | 200 |

TABLE 56
Results from second dephosphorylation reaction:

| | | Conversion |
|---|---|---|
| | | (%) to |
| Gene name | SEQ ID NO | product* |
| Wild type phosphatase | 95 | 92.63 |
| (Pandalus borealis) | | |
| protein sequence - ‘SAP’ | | |

*Conversion (%) to product - % area of SEQ ID NO: 97 peak vs. SEQ ID NO: 98 peak at wavelength 258 nm

### Example 28: Specific Chain Cleavage by Endonuclease for Release of Segment or Product Oligoneucleotide Sequence Having 2′MOE Base Modifications and Phosphorothioate Linkages

Wild-type endonuclease (SEQ ID NO:100) was fused at the N-terminus to CBD. Genes were synthesised, cloned into pET28a and protein encoding the gene produced in E. coli BL21(DE3) A1 using standard cloning, expression and extraction methods. Proteins were purified by binding to beaded cellulose followed by cleavage with TEV protease using standard methods and used directly.

Reaction was set up as per table 58, with addition of endonuclease last. Reaction was incubated at 65° C. for 16 hours. Reaction was then quenched by heating to 95° C. for 5 min, centrifuged at 4000×g for 15 min and 10 μL of supernatant transferred to an HPLC vial. Reaction was then analysed by HPLC and LCMS for the presence of SEQ ID NO: 101 and ACTIA (specific modifications set out in table 57).

TABLE 57

| SEQ ID NO | Sequence |
|---|---|
| 99 | ACT/ideoxyl/A*G*A*5mC(MOE)*5mC(MOE)*A(MOE)*5mC(MOE)*G(MOE) |
| 101 | /5phos/G*A*5mC(MOE)*5mC(MOE)*A(MOE)*5mC(MOE)*G(MOE) |
| N/A | ACTIA |

TABLE 58

| | Final | |
|---|---|---|
| | concentration | |
| | in reaction | Volume per |
| | (mM) | reaction (μL) |
| Oligonucleotide: (2′MOE, PS) - | 20 | 2 |
| SEQ ID NO: 99 | | |
| Endonuclease - SEQ ID NO: 100 | | 88 |
| Buffer (50 mM Sodium chloride, 10 mM | | 10 |
| Tris-HCl, 10 mM Magnesium Chloride, | | |
| 100 μg/mL BSA at pH 7.9) | | |
| Final reaction volume: | | 100 |

TABLE 59
Results from reaction:

| | | Conversion (%) |
|---|---|---|
| Gene name | SEQ ID NO | to product* |
| Wild type Archaeoglobus fulgidus | 100 | 47.69 |
| endonuclease V endonuclease | | |

*Conversion (%) to product - % area of SEQ ID NO: 100 peak vs. SEQ ID NO: 101 and ACTIA peak at wavelength 258 nm

### Overall Conclusions

The inventors have shown that it is possible to synthesize oligonucleotides enzymatically in solution, including oligonucleotides with a range of therapeutically relevant chemical modifications, starting from simple water and air stable nucleoside derivatives, firstly by using an RNA ligase enzyme to produce short oligonucleotide segments and secondly by assembling the short segments on a complementary template. The segments can then be ligated together to produce a product oligonucleotide which can be separated from both impurities and its complementary template in an efficient process that is scalable and suitable for large scale therapeutic oligonucleotide manufacture.

By synthesizing oligonucleotides in solution, the inventors have avoided the scale up constraints imposed by solid phase methods. In using the inherent properties of DNA to recognise complementary sequences specifically and bind complementary sequences with an affinity that reflects both the fidelity of the complementary sequence and the length of the complementary sequence, the inventors have been able to produce oligonucleotides of high purity without the need for chromatography, which both improves the efficiency of the production process and the scalability of the process. By recovering the template in an unchanged state during the separation process the inventors are able to reuse the template for further rounds of synthesis and so have avoided the economic consequences of having to make one equivalent of template for every equivalent of product oligonucleotide formed.

Finally, although wild type ligases are known to ligate normal DNA and RNA effectively, we have shown that modifications to DNA or RNA result in decreased ligation efficiency and multiple modifications to the DNA or RNA are additive in their effect on decreasing the efficiency of ligation which can, in some cases render the DNA or RNA ligase completely ineffective. We have shown that by appropriate mutation and evolution of DNA and RNA ligases, ligation efficiency can be restored and appropriately modified DNA and RNA ligases are effective catalysts for synthesizing oligonucleotides which contain multiple modifications.

### SEQUENCE LISTING

| SEQ ID NO | Sequence Identifier |
|---|---|
| 1 | Example 1 desired product oligonucleotide sequence (“target”) |
| 2 | Example 1-4, 6, 10 and 11 template oligonucleotide sequence |
| 3 | Wild-type T4 DNA ligase protein sequence |
| 4 | Wild-type T4 DNA ligase protein sequence (when fused to CBD) |
| 5 | Example 2 target sequence |
| 6 | Wild-type Enterobacteria phage CC31 DNA ligase protein sequence |
| 7 | Wild-type Enterobacteria phage CC31 DNA ligase protein sequence (when fused to |
| | CBD) |
| 8 | Wild-type Shigella phage Shf125875 DNA ligase protein sequence |
| 9 | Wild-type Shigella phage Shf125875 DNA ligase protein sequence (when fused to |
| | CBD) |
| 10 | Mutant ligase (T4 backbone) protein sequence |
| 11 | Mutant ligase (T4 backbone) protein sequence |
| 12 | Mutant ligase (T4 backbone) protein sequence |
| 13 | Mutant ligase (T4 backbone) protein sequence |
| 14 | Mutant ligase (T4 backbone) protein sequence |
| 15 | Mutant ligase (T4 backbone) protein sequence |
| 16 | Mutant ligase (T4 backbone) protein sequence |
| 17 | Mutant ligase (T4 backbone) protein sequence |
| 18 | Mutant ligase (T4 backbone) protein sequence |
| 19 | Mutant ligase (T4 backbone) protein sequence |
| 20 | Mutant ligase (T4 backbone) protein sequence |
| 21 | Mutant ligase (T4 backbone) protein sequence |
| 22 | Mutant ligase (T4 backbone) protein sequence |
| 23 | Mutant ligase (Enterobacteria phage CC31 backbone-clone A4) protein sequence |
| 24 | Mutant ligase (Enterobacteria phage CC31 backbone) protein sequence |
| 25 | Mutant ligase (Enterobacteria phage CC31 backbone) protein sequence |
| 26 | Mutant ligase (Enterobacteria phage CC31 backbone) protein sequence |
| 27 | Mutant ligase (Enterobacteria phage CC31 backbone) protein sequence |
| 28 | Mutant ligase (Shigella phage Shf125875 backbone) protein sequence |
| 29 | Wild-type Chlorella ligase protein sequence |
| 30 | Example 5, 8 and 9 template oligonucleotide sequence |
| 31 | Example 5 template oligonucleotide sequence |
| 32 | Example 5 template oligonucleotide sequence |
| 33 | Example 5 template oligonucleotide sequence |
| 34 | Example 5 template oligonucleotide sequence |
| 35 | Example 5 template oligonucleotide sequence |
| 36 | Example 5 template oligonucleotide sequence |
| 37 | Example 5 template oligonucleotide sequence |
| 38 | Example 5 template oligonucleotide sequence |
| 39 | Example 5 template oligonucleotide sequence |
| 40 | Example 5 template oligonucleotide sequence |
| 41 | Example 5 template oligonucleotide sequence |
| 42 | Example 5 template oligonucleotide sequence |
| 43 | Example 5 template oligonucleotide sequence |
| 44 | Example 5 template oligonucleotide sequence |
| 45 | Example 5 template oligonucleotide sequence |
| 46 | Example 14 “20 mer” oligonucleotide sequence |
| 47 | Example 7 template oligonucleotide sequence |
| 48 | Paramecium bursaria Chlorella virus NE-JV-4 ligase |
| 49 | Paramecium bursaria Chlorella virus NYs1 ligase |
| 50 | Paramecium bursaria Chlorella virus NE-JV-1 ligase |
| 51 | Acanthocystis turfacea Chlorella virus Canal-1 ligase |
| 52 | Acanthocystis turfacea Chlorella virus Br0604L ligase |
| 53 | Acanthocystis turfacea Chlorella virus NE-JV-2 ligase |
| 54 | Acanthocystis turfacea Chlorella virus TN603.4.2 ligase |
| 55 | Acanthocystis turfacea Chlorella virus GM0701.1 ligase |
| 56 | Synechococcus phage S-CRM01 ligase |
| 57 | marine sediment metagenome ligase |
| 58 | Mycobacterium tuberculosis (strain ATCC 25618/H37Rv) ligase |
| 59 | Enterococcus faecalis (strain ATCC 700802/V583) ligase |
| 60 | Haemophilus influenzae (strain ATCC 51907/DSM 11121/KW20/Rd) ligase |
| 61 | Staphylococcus aureus ligase |
| 62 | Streptococcus pneumoniae (strain P1031) ligase |
| 63 | Wild-type Enterobacteria phage T4 RNA ligase 1 protein sequence-‘Rnl1’ |
| 64 | Wild-type Enterobacteria phage T4 RNA ligase 2 protein sequence-‘Rnl2’ |
| 65 | Wild-type Aeromonas virus Aeh1 ligase protein sequence-‘RNA3’ |
| 66 | Wild-type Klebsiella phage JD18 ligase protein sequence-‘RNA6’ |
| 67 | Wild-type Stenotrophomonas phage IME13 ligase protein sequence-‘RNA8’ |
| 68 | Wild-type Escherichia phage JSE ligase protein sequence-‘RNA9’ |
| 69 | Wild-type Acinetobacter phage Acj61 ligase protein sequence-‘RNA16’ |
| 70 | Wild-type Vibrio phage VH7D ligase protein sequence-‘RNA20’ |
| 71 | Wild-type Escherichia phage JS98 ligase protein sequence-‘RNA22’ |
| 72 | Wild-type Escherichia phage vB_EcoM_112 ligase protein sequence-‘RNA23’ |
| 73 | Wild-type Aeromonas phage CC2 ligase protein sequence-‘RNA28’ |
| 74 | Wild-type Enterobacteria phage RB69 ligase protein sequence-‘RNA29’ |
| 75 | Wild-type Acinetobacter phage Ac42 ligase protein sequence-‘RNA31 |
| 76 | Wild-type Pectobacterium phage CBB ligase protein sequence-‘RNA33’ |
| 77 | Wild-type Aeromonas phage phiAS5 ligase protein sequence-‘RNA35 |
| 78 | Wild-type Salmonella phage vB_SenMS16 ligase protein sequence-‘RNA36 |
| 79 | Wild-type Vibrio phage KVP40 ligase protein sequence-‘RNA39’ |
| 80 | Wild-type Campylobacter virus CP220 ligase protein sequence-‘RNA49’ |
| 81 | Wild-type Pseudomonas phage phiPMW ligase protein sequence-‘RNA59’ |
| 82 | Wild-type Pseudomonas phage vB_PaeM_C2-10_Ab1 ligase protein sequence-‘RNA61’ |
| 83 | Wild-type Butyrivibrio proteoclasticus ligase protein sequence-‘RNA82’ |
| 84 | Example 15 and 16 primer oligonucleotide sequence |
| 85 | Example 15 and 16 product oligonucleotide sequence |
| 86 | Example 17 primer oligonucleotide sequence |
| 87 | Example 17 product oligonucleotide sequence |
| 88 | Mutant ligase (Stenotrophomonas phage IME13 backbone) protein sequence |
| 89 | Mutant ligase (Stenotrophomonas phage IME13 backbone) protein sequence |
| 90 | Mutant ligase (Stenotrophomonas phage IME13 backbone) protein sequence |
| 91 | Mutant ligase (Stenotrophomonas phage IME13 backbone) protein sequence |
| 92 | Mutant ligase (Stenotrophomonas phage IME13 backbone) protein sequence |
| 93 | Example 26 product oligonucleotide sequence |
| 94 | Example 26 product oligonucleotide sequence |
| 95 | Wild type phosphatase (Pandalus borealis) protein sequence-‘SAP’ |
| 96 | Example 27 product oligonucleotide sequence |
| 97 | Example 27 product oligonucleotide sequence |
| 98 | Example 27 product oligonucleotide sequence |
| 99 | Example 28 product oligonucleotide sequence |
| 100 | Wild type Archaeoglobus fulgidus endonuclease V endonuclease |
| 101 | Example 28 product oligonucleotide sequence |

SEQ ID NO: 1
GGCCAAACCTCGGCTTACCT
SEQ ID NO: 2
TTTAGGTAAGCCGAGGTTTGGCC
SEQ ID NO: 3
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFQSLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEVIDVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 4
GSILKILNEIASIGSTKQKQAILEKNKDNELLKRVYRLTYSRGLQYYIKKWPKPGIATQSFGMLTLTDMLDFIEFTL
ATRKLTGNAAIEELTGYITDGKKDDVEVLRRVMMRDLECGASVSIANKVWPGLIPEQPQMLASSYDEKGINKNIK
FPAFAQLKADGARCFAEVRGDELDDVRLLSRAGNEYLGLDLLKEELIKMTAEARQIHPEGVLIDGELVYHEQVKK
EPEGLDFLFDAYPENSKAKEFAEVAESRTASNGIANKSLKGTISEKEAQCMKFQVWDYVPLVEIYSLPAFRLKYDV
RDSKLEQMTSGYDKVILIENQVVNNLDEAKVIYKKYIDQGLEGIILKNIDGLWENARSKNLYKFKEVIDVDLKIVGI
YPHRKDPTKAGGFILESECGKIKVNAGSGLKDKAGVKSHELDRTRIMENQNYYIGKILECECNGWLKSDGRTDY
VKLFLPIAIRLREDKTKANTFEDVFGDFHEVTGL
SEQ ID NO: 5
GGC CAA ACC UCG GCU UAC CU
SEQ ID NO: 6
MILDIINEIASIGSTKEKAIRRHKDNELLKRVFRMTYDGLKQYYIKKWDTRPKGDIHLTLEDMLYLLEEKLAKRV
VTGNAAKEKLEIALSQTSDADAEVVKKVLLRDLRCGASRSIANKVWKNLIPEQPQMLASSYDEKGIEKNIKFPAFA
QLKADGARAFAEVRGDELDDVKILSRAGNEYLGLDLLKQQLIEMTKEARERHPGGVMIDGELVYHASTLPAGPLD
DIFGDLPELSKAKEFKEESRTMSNGLANKSLKGTISAKEAAGMKFQVWDYVPLDVVYSEGKQSGFAYDVRFRALE
LMVQGYSQMILIENHIVHNLDEAKVIYRKYVDEGLEGIILKNIGAFWENTRSKNLYKFKEVITIDLRIVDIYEHSKQ
PGKAGGFYLESECGLIKVKAGSGLKDKPGKDAHELDRTRIWENKNDYIGGVLESECNGWLAAEGRTDYVKLFLPI
AIKMRRDKDVANTFADIWGDFHEVTGL
SEQ ID NO: 7
GSILDIINEIASIGSTKEKEAIIRRHKDNELLKRVFRMTYDGKLQYYIKKWDTRPKGDIHLTLEDMLYLLEEKLAKR
VVTGNAAKEKLEIALSQTSDADAEVVKKVLLRDLRCGASRSIANKVWKNLIPEQPQMLASSYDEKGIEKNIKFPAF
AQLKADGARAFAEVRGDELDDVKILSRAGNEYLGLDLLKQQLIEMTKEARERHPGGVMIDGELVYHASTLPAGPL
DDIFGDLPELSKAKEFKEESRTMSNGLANKSLKGTISAKEAAGMKFQVWDYVPLDVVYSEGKQSGFAYDVRFRA
LELMVQGYSQMILIENHIVHNLDEAKVIYRKYVDEGLEGIILKNIGAFWENTRSKNLYKFKEVITIDLRIVDIYEHS
KQPGKAGGFYLESECGLIKVKAGSGLKDKPGKDAHELDRTRIWENKNDYIGGVLESECNGWLAAEGRTDYVKLF
LPIAIKMRRDKDVANTFADIWGDFHEVTGL
SEQ ID NO: 8
MILDILNQIAAIGSTKTKQEILKKNKDNKLLERVYRLTYARGIQYYIKKWPGPGERSQAYGLLELDDMLDFIEFTLA
TRKLTGNAAIKELMGYIADGKPDDVEVLRRVMMRDLEVGASVSIANKVWPGLIQLQPQMLASAYDEKLITKNIK
WPAFAQLKADGARCFAEVRDDGVQFFSRAGNEYHGLTLLADELMEMTKEARERHPNGVLIDGELVYHSFDIKKA
VSSGNDLSFLFGDNEESEEVQVADRSTSNGLANKSLQGTISPKEAEGMVLQAWDYVPLDEVYSDGKIKGQKYDV
RFAALENMAEGFKRIEPIENQLVHNLDEAKVVYKKYVDQGLEGIILKNRDSYWENKRSKNLIKFKEVIDIALEVVG
YYEHSKDPNKLGGVELVSRCRRITTDCGSGFKDTTHKTVDGVKVLIPLDERHDLDRERLMAEAREGKLIGRIADC
ECNGWVHSKGREGTVGIFLPIIKGFRFDKTEADSFEDVFGPWSQTGL
SEQ ID NO: 9
GSILDILNQIAAIGSTKTKQEILKKNKDNKLLERVYRLTYARGIQYYIKKWPGPGERSQAYGLLELDDMLDFIEFTL
ATRKLTGNAAIKELMGYIADGKPDDVEVLRRVMMRDLEVGASVSIANKVWPGLIQLQPQMLASAYDEKLITKNIK
WPAFAQLKADGARCFAEVRDDGVQFFSRAGNEYHGLTLLADELMEMTKEARERHPNGVLIDGELVYHSFDIKKA
VSSGNDLSFLFGDNEESEEVQVADRSTSNGLANKSLQGTISPKEAEGMVLQAWDYVPLDEVYSDGKIKGQKYDV
RFAALENMAEGFKRIEPIENQLVHNLDEAKVVYKKYVDQGLEGIILKNRDSYWENKRSKNLIKFKEVIDIALEVVG
YYEHSKDPNKLGGVELVSRCRRITTDCGSGFKDTTHKTVDGVKVLIPLDERHDLDRERLMAEAREGKLIGRIADC
ECNGWVHSKGREGTVGIFLPIIKGFRFDKTEADSFEDVFGPWSQTGL
SEQ ID NO: 10
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKRVIDVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 11
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKGVIDVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 12
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKKVIDVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 13
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEVILVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 14
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEVIKVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 15
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEVIQVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 16
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEVIVVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 17
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEVIRVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 18
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEAIDVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 19
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKEKIDVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 20
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKRVIVVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 21
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKKVIEVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 22
MILKILNEIASIGSTKQKQAILEKNKDNELLKRVYRLTYSRGLQYYIKKWPKPGIATQSFGMLTLTDMLDFIEFTLA
TRKLTGNAAIEELTGYITDGKKDDVEVLRRVMMRDLECGASVSIANKVWPGLIPEQPQMLASSYDEKGINKNIKF
PAFAQLKADGARCFAEVRGDELDDVRLLSRAGNEYLGLDLLKEELIKMTAEARQIHPEGVLIDGELVYHEQVKKE
PEGLDFLFDAYPENSKAKEFAEVAESRTASNGIANKSLKGTISEKEAQCMKFQVWDYVPLVEIYSLPAFRLKYDVR
FSKLEQMTSGYDKVILIENQVVNNLDEAKVIYKKYIDQGLEGIILKNIDGLWENARSKNLYKFKKVIHVDLKIVGIY
PHRKDPTKAGGFILESECGKIKVNAGSGLKDKAGVKSHELDRTRIMENQNYYIGKILECECNGWLKSDGRTDYV
KLFLPIAIRLREDKTKANTFEDVFGDFHEVTGL
SEQ ID NO: 23
MILDIINEIASIGSTKEKEAIIRRHKDNELLKRVFRMTYDGKLQYYIKKWDTRPKGDIHLTLEDMLYLLEEKLAKRV
VTGNAAKEKLEIALSQTSDADAEVVKKVLLRDLRCGASRSIANKVWKNLIPEQPQMLASSYDEKGIEKNIKFPAFA
QLKADGARAFAEVRGDELDDVKILSRAGNEYLGLDLLKQQLIEMTKEARERHPGGVMIDGELVYHASTLPAGPLD
DIFGDLPELSKAKEFKEESRTMSNGLANKSLKGTISAKEAAGMKFQVWDYVPLDVVYSEGKQSGFAYDVRFRALE
LMVQGYSQMILIENHIVHNLDEAKVIYRKYVDEGLEGIILKNIGAFWENTRSKNLYKFKRVIVIDLRIVDIYEHSKQ
PGKAGGFYLESECGLIKVKAGSGLKDKPGKDAHELDRTRIWENKNDYIGGVLESECNGWLAAEGRTDYVKLFLPI
AIKMRRDKDVANTFADIWGDFHEVTGL
SEQ ID NO: 24
MILDIINEIASIGSTKEKEAIIRRHKDNELLKRVFRMTYDGKLQYYIKKWDTRPKGDIHLTLEDMLYLLEEKLAKRV
VTGNAAKEKLEIALSQTSDADAEVVKKVLLRDLRCGASRSIANKVWKNLIPEQPQMLASSYDEKGIEKNIKFPAFA
QLKADGARAFAEVRGDELDDVKILSRAGNEYLGLDLLKQQLIEMTKEARERHPGGVMIDGELVYHASTLPAGPLD
DIFGDLPELSKAKEFKEESRTMSNGLANKSLKGTISAKEAAGMKFQVWDYVPLDVVYSEGKQSGFAYDVRFRALE
LMVQGYSQMILIENHIVHNLDEAKVIYRKYVDEGLEGIILKNIGAFWENTRSKNLYKFKKVIKIDLRIVDIYEHSKQ
PGKAGGFYLESECGLIKVKAGSGLKDKPGKDAHELDRTRIWENKNDYIGGVLESECNGWLAAEGRTDYVKLFLPI
AIKMRRDKDVANTFADIWGDFHEVTGL
SEQ ID NO: 25
MILDIINEIASIGSTKEKEAIIRRHKDNELLKRVFRMTYDGKLQYYIKKWDTRPKGDIHLTLEDMLYLLEEKLAKRV
VTGNAAKEKLEIALSQTSDADAEVVKKVLLRDLRCGASRSIANKVWKNLIPEQPQMLASSYDEKGIEKNIKFPAFA
QLKADGARAFAEVRGDELDDVKILSRAGNEYLGLDLLKQQLIEMTKEARERHPGGVMIDGELVYHASTLPAGPLD
DIFGDLPELSKAKEFKEESRTMSNGLANKSLKGTISAKEAAGMKFQVWDYVPLDVVYSEGKQSGFAYDVRFRALE
LMVQGYSQMILIENHIVHNLDEAKVIYRKYVDEGLEGIILKNIGAFWENTRSKNLYKFKGVIFIDLRIVDIYEHSKQ
PGKAGGFYLESECGLIKVKAGSGLKDKPGKDAHELDRTRIWENKNDYIGGVLESECNGWLAAEGRTDYVKLFLPI
AIKMRRDKDVANTFADIWGDFHEVTGL
SEQ ID NO: 26
MILDIINEIASIGSTKEKEAIIRRHKDNELLKRVFRMTYDGKLQYYIKKWDTRPKGDIHLTLEDMLYLLEEKLAKRV
VTGNAAKEKLEIALSQTSDADAEVVKKVLLRDLRCGASRSIANKVWKNLIPEQPQMLASSYDEKGIEKNIKFPAFA
QLKADGARAFAEVRGDELDDVKILSRAGNEYLGLDLLKQQLIEMTKEARERHPGGVMIDGELVYHASTLPAGPLD
DIFGDLPELSKAKEFKEESRTMSNGLANKSLKGTISAKEAAGMKFQVWDYVPLDVVYSEGKQSGFAYDVRFRALE
LMVQGYSQMILIENHIVHNLDEAKVIYRKYVDEGLEGIILKNIGAFWENTRSKNLYKFKGVILIDLRIVDIYEHSKQ
PGKAGGFYLESECGLIKVKAGSGLKDKPGKDAHELDRTRIWENKNDYIGGVLESECNGWLAAEGRTDYVKLFLPI
AIKMRRDKDVANTFADIWGDFHEVTGL
SEQ ID NO: 27
MILDIINEIASIGSTKEKEAIIRRHKDNELLKRVFRMTYDGKLQYYIKKWDTRPKGDIHLTLEDMLYLLEEKLAKRV
VTGNAAKEKLEIALSQTSDADAEVVKKVLLRDLRCGASRSIANKVWKNLIPEQPQMLASSYDEKGIEKNIKFPAFA
QLKADGARAFAEVRGDELDDVKILSRAGNEYLGLDLLKQQLIEMTKEARERHPGGVMIDGELVYHASTLPAGPLD
DIFGDLPELSKAKEFKEESRTMSNGLANKSLKGTISAKEAAGMKFQVWDYVPLDVVYSEGKQSGFAYDVRFRALE
LMVQGYSQMILIENHIVHNLDEAKVIYRKYVDEGLEGIILKNIGAFWENTRSKNLYKFKRVIFIDLRIVDIYEHSKQ
PGKAGGFYLESECGLIKVKAGSGLKDKPGKDAHELDRTRIWENKNDYIGGVLESECNGWLAAEGRTDYVKLFLPI
AIKMRRDKDVANTFADIWGDFHEVTGL
SEQ ID NO: 28
MILDILNQIAAIGSTKTKQEILKKNKDNKLLERVYRLTYARGIQYYIKKWPGPGERSQAYGLLELDDMLDFIEFTLA
TRKLTGNAAIKELMGYIADGKPDDVEVLRRVMMRDLEVGASVSIANKVWPGLIQLQPQMLASAYDEKLITKNIK
WPAFAQLKADGARCFAEVRDDGVQFFSRAGNEYHGLTLLADELMEMTKEARERHPNGVLIDGELVYHSFDIKKA
VSSGNDLSFLFGDNEESEEVQVADRSTSNGLANKSLQGTISPKEAEGMVLQAWDYVPLDEVYSDGKIKGQKYDV
RFAALENMAEGFKRIEPIENQLVHNLDEAKVVYKKYVDQGLEGIILKNRDSYWENKRSKNLIKFKRVIVIALEVVG
YYEHSKDPNKLGGVELVSRCRRITTDCGSGFKDTTHKTVDGVKVLIPLDERHDLDRERLMAEAREGKLIGRIADC
ECNGWVHSKGREGTVGIFLPIIKGFRFDKTEADSFEDVFGPWSQTGL
SEQ ID NO: 29
MAITKPLLAATLENIEDVQFPCLATPKIDGIRSVKQTQMLSRTFKPIRNSVMNRLLTELLPEGSDGEISIEGATFQD
TTSAVMTGHKMYNAKFSYYWFDYVTDDPLKKYIDRVEDMKNYITVHPHILEHAQVKIIPLIPVEINNITELLQYER
DVLSKGFEGVMIRKPDGKYKFGRSTLKEGILLKMKQFKDAEATIISMTALFKNTNTKTKDNFGYSKRSTHKSGKV
EEDVMGSIEVDYDGVVFSIGTGFDADQRRDFWQNKESYIGKMVKFKYFEMGSKDCPRFPVFIGIRHEEDR
SEQ ID NO: 30
TTTGGTGCGAAGCAGACTGAGGC
SEQ ID NO: 31
TTTGGTGCGAAGCAGAGTGAGGC
SEQ ID NO: 32
TTTGGTGCGAAGCAGATTGAGGC
SEQ ID NO: 33
TTTGGTGCGAAGCAGAATGAGGC
SEQ ID NO: 34
TTTGGTGCGAAGCAGTCTGAGGC
SEQ ID NO: 35
TTTGGTGCGAAGCAGTGTGAGGC
SEQ ID NO: 36
TTTGGTGCGAAGCAGTTTGAGGC
SEQ ID NO: 37
TTTGGTGCGAAGCAGTATGAGGC
SEQ ID NO: 38
TTTGGTGCGAAGCAGCCTGAGGC
SEQ ID NO: 39
TTTGGTGCGAAGCAGCGTGAGGC
SEQ ID NO: 40
TTTGGTGCGAAGCAGCTTGAGGC
SEQ ID NO: 41
TTTGGTGCGAAGCAGCATGAGGC
SEQ ID NO: 42
TTTGGTGCGAAGCAGGCTGAGGC
SEQ ID NO: 43
TTTGGTGCGAAGCAGGGTGAGGC
SEQ ID NO: 44
TTTGGTGCGAAGCAGGTTGAGGC
SEQ ID NO: 45
TTTGGTGCGAAGCAGGATGAGGC
SEQ ID NO: 46
GCCUCAGTCTGCTTCGCACC
SEQ ID NO: 47
TTTGGTGCGAAGCAGAAGGTAAGCCGAGGTTTGGCC
SEQ ID NO: 48
MAITKPLLAATLENIEDVQFPCLATPKIDGIRSVKQTQMLSRTFKPIRNSVMNRLLTELLPEGSDGEISIEGATFQD
TTSAVMTGHKMYNAKFSYYWFDYVTDDPLKKYSDRVEDMKNYITAHPHILDHEQVKIIPLIPVEINNITELLQYE
RDVLSKGFEGVMIRKPDGKYKFGRSTLKEGILLKMKQFKDAEATIISMTALFKNTNTKTKDNFGYSKRSTHKNGK
VEEDVMGSIEVDYDGVVFSIGTGFDADQRRDFWQNKESYIGKMVKFKYFEMGSKDCPRFPVFIGIRHEEDH
SEQ ID NO: 49
MTIAKPLLAATLENLDDVKFPCLVTPKIDGIRSLKQQHMLSRTFKPIRNSVMNKLLSELLPEGADGEICIEDSTFQA
TTSAVMTGHKVYDEKFSYYWFDYVVDDPLKSYTDRVNDMKKYVDDHPHILEHEQVKIIPLIPVEINNIDELSQYE
RDVLAKGFEGVMIRRPDGKYKFGRSTLKEGILLKMKQFKDAEATIISMSPRLKNTNAKSKDNLGYSKRSTHKSGK
VEEETMGSIEVDYDGVVFSIGTGFDDEQRKHFWENKDSYIGKLLKFKYFEMGSKDAPRFPVFIGIRHEEDC
SEQ ID NO: 50
MTAIQKPLLAASFKKLTVADVKYPVFATPKLDGIRALKIDGAFVSRTFKPIRNRAIADALQDLLPNGSDGEILSGST
FQDASSAVMTAKAGIGANTIFYWFDYVKDDPNKPYLDRMTDMENYLKERPEILNDDRIKIVPLIPKKIETKDELD
TFEKICLDQGFEGVMIRSGAGKYKFGRSTEKEGILIKIKQFEDDEAVVIGFTPMQTNTNDKSMNELGDMKRSSHK
DGKVNLDTLGALEVDWNGITFSIGTGFDHALRDKLWSERDKLIGKIVKFKYFAQGVKTAPRFPVFIGFRDPDDM
SEQ ID NO: 51
MAIQKPLLAASLKKMSVGDLTFPVFATPKLDGIRALKVGGTIVSRTFKPVRNSAISEVLASILPDGSDGEILSGKTF
QESTSTVMTADAGLGSGTMFFWFDYVKDDPNKGYLDRIADMKSFTDRHPEILKDKRVTIVPLFPKKIDTTEELHE
FEKWCLDQGFEGVMVRNAGGKYKFGRSTEKEQILVKIKQFEDDEAVVIGVSALQTNTNDKKLNQLGEMRRTSH
QDGKVELEMLGALDVDWNGIRFSIGTGFDRDTRVDLWKRREGVIGKIVKFKYFSQGIKTAPRFPVFLGFRDKDDM
SEQ ID NO: 52
MAIQKPLLAASLKKLSVDDLTFPVYATPKLDGIRALKIDGTLVSRTFKPIRNTTISKVLTSLLPDGSDGEILSGKTFQ
DSTSTVMSADAGIGSGTTFFWFDYVKDDPNKGYLDRIADIKKFIDCRPEILKDSRVIIVPLFPKKIDTAEELNVFEK
WCLDQGFEGVMVRNAGGKYKFGRSTEKEQILVKIKQFEDDEAVVIGVSALQTNTNDKKVNELGEMRRTSHQDG
KVDLDMLGALDVDWNGIRFGIGTGFDKDTREDLWKRRDSIIGKIVKFKYFSQGVKTAPRFPVFLGFRDKNDM
SEQ ID NO: 53
MAIQKPLLAASLKKLSVDDLTFPVYATPKLDGIRALKIDGTIVSRTFKPIRNTTISNVLMSLLPDGSDGEILSGKTF
QDSTSTVMSADAGIGSGTTFFWFDYVKDDPDKGYLDRIADMKKFVDSHPEILKDRRVTIVPLIPKKIDTVEELNV
FEQWCLDQGFEGVMVRNAGGKYKFGRSTEKEQILVKIKQFEDDEAVVIGVSALQTNVNDKKMNELGDMRRTSH
KDGKIDLEMLGALDVEWNGIRFGIGTGFDKDTREDLWKKRDSIIGKVVKFKYFSQGIKTAPRFPVFLGFRDENDM
SEQ ID NO: 54
MAIQKPLLAASLKKMSVDNLTFPVYATPKLDGIRALKIDGTLVSRTFKPIRNTTISKVLASLLPDGSDGEILSGKTF
QDSTSTVMTTDAGIGSDTTFFWFDYVKDDPDKGYLDRIADMKTFVDQHPEILKDSCVTIVPLFPKKIDTPEELHV
FEKWCLDQGFEGVMVRTAGGKYKFGRSTEKEQILVKIKQFEDDEAVVIGVSALQTNTNDKKLNQLGEMRRTSH
QDGKVDLDMLGALDVDWNGIRFSIGTGFDKDTREDLWKQRDSIVGKVVKFKYFSQGIKTAPRFPVFLGFRDENDM
SEQ ID NO: 55
MAIQKPLLAASLKKMSVDDLTFPVYTTPKLDGIRALKIDGTLVSRTFKPVRNSAISEVLASLLPDGSDGEILSGKTF
QDSTSTVMTTDAGIGSDTTFFWFDYVKDDPNKGYLDRIADMKTFIDQHPEMLKDNHVTIVPLIPKKIDTVEELNI
FEKWCLDQGFEGVMVRNAGGKYKFGRSTEKEQILVKIKQFEDDEAVVIGVSALQTNTNDKKLNQLGEMRRTSH
QDGKIDLEMLGALDVDWNGIRFSIGTGFDRDTRVDLWKRRDGIVGRTIKFKYFGQGIKTAPRFPVFLGFRDKDDM
SEQ ID NO: 56
MLAGNFDPKKAKFPYCATPKIDGIRFLMVNGRALSRTFKPIRNEYIQKLLSKHLPDGIDGELTCGDTFQSSTSAIM
RIAGEPDFKAWIFDYVDPDSTSILPFIERFDQISDIIYNGPIPFKHQVLGQSILYNIDDLNRYEEACLNEGYEGVML
RDPYGTYKFGRSSTNEGILLKVKRFEDAEATVIRIDEKMSNQNIAEKDNFGRTKRSSCLDGMVPMETTGALFVRN
SDGLEFSIGSGLNDEMRDEIWKNKSSYIGKLVKYKYFPQGVKDLPRHPVFLGFRDPDDM
SEQ ID NO: 57
MDAHELMKLNEYAERQNQKQKKQITKPMLAASLKDITQLDYSKGYLATQKLDGIRALMIDGKLVSRTFKPIRNN
HIREMLEDVLPDGADGEIVCPGAFQATSSGVMSANGEPEFIYYMFDYVKDDITKEYWRRTQDMVQWLINQGPT
RTPGLSKLKLLVPTLIKNYDHLKTYETECIDKGFEGVILRTPDSPYKCGRSTAKQEWLLKLKRFADDEAVVIGFTEK
MHNDNEATKDKFGHTVRSSHKENKRPAGTLGSLIVRDIKTEIEFEIGTGFDDELRQKIWDARPEWDGLCVKYKH
FAISGVKEKPRFPSFIGVRDVEDM
SEQ ID NO: 58
MSSPDADQTAPEVLRQWQALAEEVREHQFRYYVRDAPIISDAEFDELLRRLEALEEQHPELRTPDSPTQLVGGA
GFATDFEPVDHLERMLSLDNAFTADELAAWAGRIHAEVGDAAHYLCELKIDGVALSLVYREGRLTRASTRGDGR
TGEDVTLNARTIADVPERLTPGDDYPVPEVLEVRGEVFFRLDDFQALNASLVEEGKAPFANPRNSAAGSLRQKDP
AVTARRRLRMICHGLGHVEGFRPATLHQAYLALRAWGLPVSEHTTLATDLAGVRERIDYWGEHRHEVDHEIDG
VVVKVDEVALQRRLGSTSRAPRWAIAYKYPPEEAQTKLLDIRVNVGRTGRITPFAFMTPVKVAGSTVGQATLHN
ASEIKRKGVLIGDTVVIRKAGDVIPEVLGPVVELRDGSEREFIMPTTCPECGSPLAPEKEGDADIRCPNARGCPGQ
LRERVFHVASRNGLDIEVLGYEAGVALLQAKVIADEGELFALTERDLLRTDLFRTKAGELSANGKRLLVNLDKAKA
APLWRVLVALSIRHVGPTAARALATEFGSLDAIAAASTDQLAAVEGVGPTIAAAVTEWFAVDWHREIVDKWRAA
GVRMVDERDESVPRTLAGLTIVVTGSLTGFSRDDAKEAIVARGGKAAGSVSKKTNYVVAGDSPGSKYDKAVELG
VPILDEDGFRRLLADGPASRT
SEQ ID NO: 59
MEQQPLTLTAATTRAQELRKQLNQYSHEYYVKDQPSVEDYVYDRLYKELVDIETEFPDLITPDSPTQRVGGKVLS
GFEKAPHDIPMYSLNDGFSKEDIFAFDERVRKAIGKPVAYCCELKIDGLAISLRYENGVFVRGATRGDGTVGENIT
ENLRTVRSVPMRLTEPISVEVRGECYMPKQSFVALNEEREENGQDIFANPRNAAAGSLRQLDTKIVAKRNLNTFL
YTVADFGPMKAKTQFEALEELSAIGFRTNPERQLCQSIDEVWAYIEEYHEKRSTLPYEIDGIVIKVNEFALQDELG
FTVKAPRWAIAYKFPPEEAETVVEDIEWTIGRTGVVTPTAVMAPVRVAGTTVSRASLHNADFIQMKDIRLNDHVI
IYKAGDIIPEVAQVLVEKRAADSQPYEMPTHCPICHSELVHLDEEVALRCINPKCPAQIKEGLNHFVSRNAMNIDG
LGPRVLAQMYDKGLVKDVADLYFLTEEQLMTLDKIKEKSANNIYTAIQGSKENSVERLIFGLGIRHVGAKAAKILA
EHFGDLPTLSRATAEEIVALDSIGETIADSVVTYFENEEVHELMAELEKAQVNLTYKGLRTEQLAEVESPFKDKTV
VLTGKLAQYTREEAKEKIENLGGKVTGSVSKKTDIVVAGEDAGSKLTKAESLGVTVWNEQEMVDALDASHF
SEQ ID NO: 60
MTNIQTQLDNLRKTLRQYEYEYHVLDNPSVPDSEYDRLFHQLKALELEHPEFLTSDSPTQRVGAKPLSGFSQIRH
EIPMLSLDNAFSDAEFNAFVKRIEDRLILLPKPLTFCCEPKLDGLAVSILYVNGELTQAATRGDGTTGEDITANIRT
IRNVPLQLLTDNPPARLEVRGEVFMPHAGFERLNKYALEHNEKTFANPRNAAAGSLRQLDPNITSKRPLVLNAYGI
GIAEGVDLPTTHYARLQWLKSIGIPVNPEIRLCNGADEVLGFYRDIQNKRSSLGYDIDGTVLKINDIALQNELGFI
SKAPRWAIAYKFPAQEELTLLNDVEFQVGRTGAITPVAKLEPVFVAGVTVSNATLHNGDEIERLNIAIGDTVVIRR
AGDVIPQIIGVLHERRPDNAKPIIFPTNCPVCDSQIIRIEGEAVARCTGGLFCAAQRKEALKHFVSRKAMDIDGVG
GKLIEQLVDRELIHTPADLFKLDLTTLTRLERMGAKSAENALNSLENAKSTTLARFIFALGIREVGEATALNLANHF
KTLDALKDANLEELQQVPDVGEVVANRIFIFWREAHNVAVVEDLIAQGVHWETVEVKEASENLFKDKTVVLTGT
LTQMGRNEAKALLQQLGAKVSGSVSSKTDFVIAGDAAGSKLAKAQELNITVLTEEEFLAQITR
SEQ ID NO: 61
MADLSSRVNELHDLLNQYSYEYYVEDNPSVPDSEYDKLLHELIKIEEEHPEYKTVDSPTVRVGGEAQASFNKVNH
DTPMLSLGNAFNEDDLRKFDQRIREQIGNVEYMCELKIDGLAVSLKYVDGYFVQGLTRGDGTTGEDITENLKTIH
AIPLKMKEPLNVEVRGEAYMPRRSFLRLNEEKEKNDEQLFANPRNAAAGSLRQLDSKLTAKRKLSVFIYSVNDFT
DFNARSQSEALDELDKLGFTTNKNRARVNNIDGVLEYIEKWTSQRESLPYDIDGIVIKVNDLDQQDEMGFTQKS
PRWAIAYKFPAEEVVTKLLDIELSIGRTGVVTPTAILEPVKVAGTTVSRASLHNEDLIHDRDIRIGDSVVVKKAGDI
IPEVVRSIPERRPEDAVTYHMPTHCPSCGHELVRIEGEVALRCINPKCQAQLVEGLIHFVSRQAMNIDGLGTKIIQ
QLYQSELIKDVADIFYLTEEDLLPLDRMGQKKVDNLLAAIQQAKDNSLENLLFGLGIRHLGVKASQVLAEKYETID
RLLTVTEAELVEIHDIGDKVAQSVVTYLENEDIRALIQKLKDKHVNMIYKGIKTSDIEGHPEFSGKTIVLTGKLHQ
MTRNEASKWLASQGAKVTSSVTKNTDVVIAGEDAGSKLTKAQSLGIEIWTEQQFVDKQNELNS
SEQ ID NO: 62
MNKRMNELVALLNRYATEYYTSDNPSVSDSEYDRLYRELVELETAYPEQVLADSPTHRVGGKVLDGFEKYSHQY
PLYSLQDAFSREELDAFDARVRKEVAHPTYICELKIDGLSISLTYEKGILVAGVTRGDGSIGENITENLKRVKDIPL
TLPEELDITVRGECYMPRASFDQVNQARQENGEPEFANPRNAAAGTLRQLDTAVVAKRNLATFLYQEASPSTRD
SQEKGLKYLEQLGFVVNPKRILAENIDEIWNFIQEVGQERENLPYDIDGVVIKVNDLASQEELGFTVKAPKWAVA
YKFPAEEKEAQLLSVDWTVGRTGVVTPTANLTPVQLAGTTVSRATLHNVDYIAEKDIRKDDTVIVYKAGDIIPAV
LRVVESKRVSEEKLDIPTNCPSCNSDLLHFEDEVALRCINPRCPAQIMEGLIHFASRDAMNITGLGPSIVEKLFAAN
LVKDVADIYRLQEEDFLLLEGVKEKSAAKLYQAIQASKENSAEKLLFGLGIRHVGSKASQLLLQYFHSIENLYQADS
EEVASIESLGGVIAKSLQTYFATEGSEILLRELKETGVNLDYKGQTVVADAALSGLTVVLTGKLERLKRSEAKSKLE
SLGAKVTGSVSKKTDLVVVGADAGSKLQKAQELGIQVRDEAWLESL
SEQ ID NO: 63
MQELFNNLMELCKDSQRKFFYSDDVSASGRTYRIFSYNYASYSDWLLPDALECRGIMFEMDGEKPVRIASRPME
KFFNLNENPFTMNIDLNDVDYILTKEDGSLVSTYLDGDEILFKSKGSIKSEQALMANGILMNINHHRLRDRLKELA
EDGFTANFEFVAPTNRIVLAYQEMKIILLNVRENETGEYISYDDIYKDATLRPYLVERYEIDSPKWIEEAKNAENIE
GYVAVMKDGSHFKIKSDWYVSLHSTKSSLDNPEKLFKTIIDGASDDLKAMYADDEYSYRKIEAFETTYLKYLDRA
LFLVLDCHNKHCGKDRKTYAMEAQGVAKGAGMDHLFGIIMSLYQGYDSQEKVMCEIEQNFLKNYKKFIPEGY
SEQ ID NO: 64
MFKKYSSLENHYNSKFIEKLYSLGLTGGEWVAREKIHGTNFSLIIERDKVTCAKRTGPILPAEDFFGYEIILKNYAD
SIKAVQDIMETSAVVSYQVFGEFAGPGIQKNVDYCDKDFYVFDIIVTTESGDVTYVDDYMMESFCNTFKFKMAPL
LGRGKFEELIKLPNDLDSVVQDYNFTVDHAGLVDANKCVWNAEAKGEVFTAEGYVLKPCYPSWLRNGNRVAIKC
KNSKFSEKKKSDKPIKAKVELSEADNKLVGILACYVTLNRVNNVISKIGEIGPKDFGKVMGLTVQDILEETSREGIT
LTQADNPSLIKKELVKMVQDVLRPAWIELVS
SEQ ID NO: 65
MQSIKELYQNLINLCVDDNTKFYFAETVTSLGTKVRIFDYHVAGYNDWIRPDAMASRGIMFEMNGEIPVRIMSR
PMDKFFNYSEVIGWEKLETAGNMKMPDLNKIAYVIDKRDGSLISTYLDISGEIKNLLLKSKASIRSNQANDASVW
LYQEDHKDLLEFCTAYAENGFTVNMEWTAPHNQIVLCYNEHQLRILNIRHNETGEYVDFGELQKDPTFVKYAAD
FFEVPGDGKAWIDEVYQMTGIEGFVVVMEDYQMFKLKTDWYVALHHTKDSINNSERLIYACAENCTDDLRQMF
RDDENSLQKIEIFDNHFRDVVMDAMKKLTEAYEKYRGMERRDYAINMNNDFKNERHWFNIAMQMFAQRPDFS
MTDEIVAVIKKYPKTFIPKGY
SEQ ID NO: 66
MLELYKNLMNLCESSEVAKFFYKDFTGPMDGKFRVFSYHYASYSEWLKPDALECRGIMFEMDGDTPIRIASRPM
EKFFNLNENPMTMGIDISDVEYIMDKADGSLVSSYVDDGYLYLKSKTSLYSDQARQASALLNSEEYSSLHQVILEL
ALDGYTVNMEFVSPNNRVVLAYQEPQLFVLNVRNNTTGEYIKYDDLYANAKIRPYLINAYGISDPTTWVEGVRAL
EGVEGYIAVLNTGQRFKVKTEWYSALHHTKDSITSNERLFASVVSANSDDLRSLFAGDEYAIKKISAFEQAYLDYL
GKSLELCQSFYDEYRGRARKDYAIAAQKATVNQRHLFGVIMNMYEGTVDVDKLLKDLERVFLKYWAGYVPKEYE
KEIEISEE
SEQ ID NO: 67
MSRTIELFNNLMSVVEKSEKGNFYFKDVITSMGTKARIFSYFIASYTDWLQDDALECRGIMFELNDKNEPVRIMA
RPMQKFFNLKENPMTIGLDLTKMIGLMEKADGSLISSYHDQGYVYLKSKAAIFSDQANKAMALLNSPAYEKLRDA
IVRAGSDFTFNMEYVGPSNRVVLPYEEEELIVLNVRHNETGQYVEFSTLLDDPLIRHRMIGVYPCPDWSKVTPEE
WEAATRAETDIEGVIGIMPDGQLFKLKTDWYSSLHRTKDSINNNKALFQSIKERASDDLRGMFSDDNAALAKIE
AFESAYIDTVAKYHKICAEVFLRFARFLIVEVFAIEAQARMKDCRYLFSIVMQQYGRDWDGELAVEKIEEHIIKEYA
TYVPMAYR
SEQ ID NO: 68
MEHKLCQQKKTTKKVLALFKNLMALCDGSDTFYYKDEITAMQTRMRIFSYLYLSKPEMWNKPDALECRGIMFEI
DDKDRPVRIASRPMEKFFNLGENDRARFKPEDVEMVMDKMDGSLVSTYIDNGYVQLKSKAALYSSQAERANGLL
YSEKYADLRAKIADIGSDYTFNFEYTAPSNRIVVEYSEPSLTLLNVRHNVTGEYVPHQMLFADAILRPHLVNALQV
DSARFSNILDEVRTAEGIEGIVARTKDGQMFKVKSEWYIGVHNIKNTSMFPNNLIYYVVESETDDLRAAYEGEPE
ALERIELFEEAFRSILRNAFTTATEFYNAHAGEDRKTYASNATIESRKHGDAQSYIFMCLMIAFDGLDYDRILASM
KTYYLRNYKKLIPADKIDW
SEQ ID NO: 69
MKELFDNLMALQDPNDISKFFYKDVVTQPGTKCRIFSYNYAAYSDWLLPGALESRGIMFELDADNQPVRVMARP
MEKFFNLEENPFTMDLDLSQLEYAMTKADGSLISTYVDQGYLYTKSKGSISSSQAIESKQLLLDINYKPLAERALE
LAKDGFTCNFEYVAPNNRIVLNYAKKDLILLNVRHNETGEYVPMAELQKDPVLRNYLIDVYPPREDIDTNEMIKEI
REMVDIEGFVFQHASGLKFKLKTEWYSNLHRVKDTLNNSEALFMVVVAGGSDDMKSLFTDDLSRTKIESFETAFL
DYLKKTSNFVFDLQRQLIGSDRKTYAIECQTILRNTDQLELFGVMMELYKGADQEQTIKNINVVFMKNYKKYVPA
GFETLKNEY
SEQ ID NO: 70
MNVQELYKNLMSLADDAEGKFFFADHLSPLGEKFRVFSYHIASYSDWLLPGALEARGIMFQLDDNDEMIRIVSRP
MEKFFNLNENPFTMELDLTTTVQLMDKADGSLISTYLSGENFALKSKTSIFSEQAVAANRYIKKPENRDLWEFCD
DCTQAGLTVNMEWCAPNNRIVLEYPEAKLVILNIRDNETGDYVSFDDIPQSALMRVKQWLVDEYDPATAHEPDF
VEKLRDTKGIEGMILRLANGQSVKIKTQWYVDLHSQKDSVNVPKKLVTTILNGNHDDLYALFADDKPTIERIREF
DSHVTKTLTNSFNAVRQFYARNRHLARKDYAIAGQKVLKPWEFGVAMIAYQKQTVEGVYESLVTAYLKRPELAIP
EKYLNGV
SEQ ID NO: 71
MIELYDNLMTLVKNSTKSKFFFKDFQSALGVNYRIFSYNYASYSDWLEDGALECRGIMFEMDENGPVRIAARPM
QKFFNLDENPLTIGLDLSQENIDLVMAKEDGSLISTFMDRQYLSVKSKGSIHSSMVHDSLRFLRLPENEAFAARLE
EITKAGYTCNLEYVSPTNRIVLAYQETNLILLNVRNNETGEYIPYAELFKDGALRKHLVKSYELSEGDFVDNIRKQE
GIEGFIFVLKDGTFFKLKTAWYSALHHTKDSINNNERLFEVVVAGGTDDLRGLFSTDSFAIEKINAFERIHLDYLE
QSLALLEAAYSQLKGRDRKDYAVTGQLILKDFPGLFSILMQAYVDGINYDTVMDQINSVFLKNHKAQIPEKYLKEI
VVE
SEQ ID NO: 72
MVLYSKHKRGYTMQELFNNLMELCKDSQRKFFYSDDVSASGRTYRIFSYNYASYSDWLLPDALECRGIMFEMDG
EKPVRIASRPMEKFFNLNENPFTMNIDLNDVDYILTKEDGSLVSTYLDGDEILFKSKGSIKSEQALMANGILMNIN
HHQLRDRLKELAEDGFTANFEFVAPTNRIVLAYQEMKIILLNVRENETGEYISYDDIYKDAILRPYLVERYEIDSPK
WVEEAKNAENIEGYVAVMKDGSHFKIKSDWYVSLHSTKSSLDNPEKLFKTIIDGASDDLKAMYADDEYSYRKIEA
FETTYLKYLDRALFLVLDCHNKHCGKDRKTYAMEAQGVAKGAGMDHLFGIIMSLYQGYDSQEKVMCEIEQNFLK
NYKKFIPEGY
SEQ ID NO: 73
MKELYNNLLKLTEEHGDCFFFRDHWSSIGNHFRVFSYHIAGLTQWMLPDALECRGIMFELINGEPYRIASRPMEK
FFNLAERQAWNLTNSGVVGLDKLELDYSNIDRYEDKADGSLMSSYHFIDPEDDNRINYMLKSKTSINSDQANDA
NRWLVNHTDLLDFIIDCEEAGYTVNLEWCSPKNQIVIMYPEESLKILNVRHRDTGEYYSNNSLIRSPVFRKYAVD
QFMFEEGTDVNTAISNMYNETGIEGYILVMRDGSRVKIKTTSYVARHKLKDSITNNKDLVIAVAQGVSDDLRQLF
LDDSLSLTKIQEFEDHVVSVAGSTYTKIREAHKACAGMERREYAISMQNTFKQDRMFFNIVMKMFQAPDLEVMP
EIMSVIIKYPDEFVPTKWK
SEQ ID NO: 74
MEKLYYNLLSLCKSSSDRKFFYSDDVSPIGKKYRIFSYNFASYSDWLLPDALECRGIMFEMDGETPLRIASRPMEK
FFNLNENPFTLSIDLNDVKYLMTKEDGSLVSTYLDGNMVRFKSKGSIKSDQAASATSILLDINHKDLADRLLELCN
DGFTANFEYVAPSNKIVLTYPEKRLILLNIRDNNTGKYIEYDDIYLDPVFRKYLVDRFEAPEGDWVPGVKSSTNIE
GYVAVMKDGSHFKLKTDWYVALHTTRDSISSPEKLFLAIMNGASDDLKAMYADDEFSFKKVELFEKAYLDFLDRS
FYICLDAYDKHKGKDRKTYAIEAQAICKGAQSPWLFGIIMNLYQGGSKEQMMTALESVFIKNHKNFIPEGY
SEQ ID NO: 75
MNELYNNLMTLIEPGKMSRFFLRDAVTPFGTRVRMFGYNYASYTDWLLPDALEARGIMFEMDENDQPIRVMAR
PMEKFFNLGENPFTIDLDLSTIEYFMDKSDGSLISSYVDNDTLFMKSKMSIGSVQAVAARQVIQDYVHRDLHDRV
LELAKDGFTCNFEYVAPDNRVVILYPERALVLLNVRNNETGEYVHIEELKRDPVLRRYLVNNYVIDPENFDQDTFV
NDIYQMVDIEGYVFRLMTGQHVKIKTEWYKMLHYAKDTINNNEALFAITVAAQLDDVRSLYSDDYALGKINKFE
EVFLGFLDTRLPILLNLHKELNGSSRKDYAIKSQTYFKQANELYLFGIFMQMFEGVPPREQLVEKLSEAFMKNYKL
FVPPEYDKVVEYDN
SEQ ID NO: 76
MRTKQIFDDLMNLTAKNDAFMWKDFVSPAGGLFRIFSYRLASYSDFLEPNALECRGSMFKVDDEGNFVGIASRT
PMKFFNAYENPFTMYDKDTLSSEIAVVMDKLDGSIISTFMDVDFVVRTKSHASLHSDHAYNSTAMLIADKELYNE
VHYAESMGYTVNMEYTSPEYRIVLPYQEDNLTVLNLRHRETGELLIGERLKEFSKILYERSVFAKHGEIDATFPMK
ETLKESIDAVRGMADIEGYVLILKDGRMCKIKTDWYCALHFTKDSINVDSRLYDAIITGASDDLKQMFSTDLYAM
KKIEKMEQLIFSCYNKLVHDVESFYEENKHLERKEYALKVQSTLPNELGMPGLAFSLYASKPVDYKGQMLKYMKD
VLVNFEV
SEQ ID NO: 77
MQSIKELYQNLVNLCVDDNTKFFFSETVTSLGTKVRIFDYHVAGYNDWIRPDAMACRGIMFEMNGEIPVRIMSR
PMDKFFNYSEVIGWEKLETHGNMKMPDLSKIKFVIDKRDGSLISTFMDVDNLLLKSKGSIRSNQANDASVWLYQ
DDQADLLEFCRAYAKEGFTVNMEWTAPHNQIVLCYTDHQLRILNIRHNETGEYVDFAELQKDSVFRKYAADFYE
VPQDGAEWIKNVYGMTGIEGYVVVMEDYQMFKLKTDWYVALHHTKDSINDSKRLISACAENATDDLRQMFRD
DPNSLAKIEVFDAKFRDVVSSAMQALTNAYSKYKALERREYAISMTNEFKNERHWFNIAMQMFSTRPDFSLADEI
VAVIKKYPEKFVPQGY
SEQ ID NO: 78
MKELFDNLMNLCNDTDESRFFYRDDISPSGLKYRIFSYNYASYSDWLLPDALECRGIMFEMIDGVPVRIASRPME
KFFNLNETPFTMNLDLSNAVHMMKKEDGSLVSSYLDGNILRFKSKSSLKSEQAYLSSAMLTSITHEALLWRLLELA
RDGFTANFEYVSPENRIVLAYQKKDLILLNIRENDTGAYVPYNEIAKDPVLRQYLVESYEIPEGDFVSDIKAMEGIE
GYVFVMDNGLRFKLKTDWYTALHHTKDSITKNDRLFEVIVNNASDDLKGLFSNDAYSLKKINKFEEVYLDYLRRS
LSFISTSYQKLRGLDRKTYAGEAKRLADAERLPFLFTILMLMFNDSMDYDTTIKKVNELFMKNYKTFIPKEYE
SEQ ID NO: 79
MTTQELYNHLMTLTEDAEGKFFFADHISPLGEKLRVFSYHIASYSDWLLPGALEARGIMFQLDEQDKMVRIVSRP
MEKFFNLNENPFTMDLDLTTTVQLMDKADGSLISTYLTGENFALKSKTSIFSEQAVAANRYIKLPENRDLWEFCD
DLTQAGCTVNMEWCAPNNRIVLEYPEAKLVILNIRDNETGDYVSFDDIPLPALMRVKKWLVDEYDPETAHVDDF
VEKLRATKGIEGMILRLANGQSVKIKTQWYVDLHSQKDSVNVPKKLVTTILNNNHDDLYALFADDKPTIDRIREF
DSHVSKTVSASFHAVSQFYVKNRHMSRKDYAIAGQKALKPWEFGVAMIAYQKKTVEGVYEALVGAYLKRPELLIP
EKYLNEA
SEQ ID NO: 80
MNYLELKNMAENLKDSNYRSTKLNNFKFYTYIFSDYKNFKENNTFFIRGLMIDSKSILNKDTLAPGISIPMPKFFNI
NENEDWLLPDSTNLEDFTIVTKYDGSLMIPYEYDGIKFRTKMSIDNDQTKLANKYIKNNPDILDLIKNNPDTQYF
FELISPLNRIVVDYNKTELKLIAELDLRTLEFKIHETNEFNFKDLNIKTLKDLKDYINTISNYEGVILQHKVTKKVYKL
KTQEYLDLHNTMTNLDLKVIYKMILEETIDDVLPKLSPEAVAYIDSVSNSVKVKLNEILDSIDSNYIKTKDLETPAL
YIKDLNIDPIAKDCLFKLCKNKLNLDDVLNQVKKSMLKYNKLRDIKVFLKWIH
SEQ ID NO: 81
MKVIEFLKNAPSITDGLASLHLELGIKAKIYEDEGLIVLNYSQIDSPKTHPIVQECRGLIIDNDLTVVARPFDRFFNY
GEALNVMPEIDWENASIFEKVDGSLIKIYFHKGRWEVATRGTAFAESECMGHGITFKELVFNALKVHDDDGFQY
LMNNAYLFRDTTYLFELTCVENRVVRHYHGYNLHFLAARDNVSGNYSEECRDWLRSPDCILYGIVKHPKRYALG
SADEALQAAKELKNLDEGFIVYQNAVPIAKIKSPAYVAVHHIRGEGLNPKRIMELVLSGEHDEYLSYFPEDRPIIQP
YVDSLLDMLNIIAVTYPRLNQATTPKAFAAAIKHAGIDKQKASVYFMARRDNKDPVQVFHGMKTTFKMDMLRK
WMMV
SEQ ID NO: 82
MPNCRIEIRRSRMEGNLNIAMYKDLIANKLVTVKHFNGMSIIKYARKVFYENLWNEHPLLLEARGHVFDQHSGD
CIVRPFEKVFNLGENGAGSFLHPKFRVRLIEKVNGFMFSVTKHNGSLIFSTTGSLTSDYVALGQKYVSNNPDDYIA
GFTYNFEICSPDDPHIVEEEEGAYLIGIRDIFTGGQLSEYILDSHALGVTDHSSVKILRPEHIECSWEHAKGLLSSC
EKEGYMVQTGLGTVKCKSTHYLGKKFIMRMGSKKVNSMYQDPAGFKQTLDEEFYPLVDFLVNEVEEVRFTEMT
DAQRRLLIETYFDMARI
SEQ ID NO: 83
MKSRILEFIKNNPDTWEEKLNEKFIRTNHNGDLVCFKYATEADFSDPLVCEARGIIIDVAQLVVVCWPFDKFFNV
QEKYAADIDWNSARVLEKIDGSMIKLFWYKGAWRFATSSTCDAKDAAIPGYNELTYADIIARAENVNEIPFEELN
KDYTYIFELVSPLSQIVVRYEMTELFFLTARNNLTGEELDTELLQFRRPRSFALKSMNECLDAALALNKGDEIEDE
GFVVVDEKHNRVKIKSPAYVAMHRLSTNKVFTVKRMAEFFCNGEDLSKLAKDFPANAHIIKYYDWQFAEMKHKA
EDMMLYSRRLYEEYDHDRKAVAMTIKDSPYAWAGFRAIGNDKDITDIMAVLAPANVEKLIVEYPEISN
SEQ ID NO: 84
ACUCCUCGGU
SEQ ID NO: 85
ACUCCUCGGUA
SEQ ID NO: 86
ACTCATCGAT
SEQ ID NO: 87
ACTCATCGATA
SEQ ID NO: 88
MSRTIELFNNLMSVVEKSEKGNFYFKDVITSMGTKARIFSHKIASYTDWLQDDALECRGIMFELNDKNEPVRIMA
RPMQKFFNLKENPMTIGLDLTKMIGLMEKADGSLISSYHDQGYVYLKSKTSIFSDQANKAMALLNSPAYEKLRDA
IVRAGSDFTFNMEYVGPSNRVVLPYEEEELIVLNVRHNETGQYVEFSTLLDDPLIRHRMIGVYPCPDWSKVTPEE
WEAATRAETDIEGVIGIMPDGQLFKLKTDWYSSLGRTKFSINNNKALHQSIKERASVALRGMFSDDNAALAKIEA
FESAYIDTVAKYHKICAEVFLRFARFLIVEVFAIEAQARMKDCRYLFSIVMQQYGRDWDGELAVEKIEEHIIKEYAT
YVPMAYR
SEQ ID NO: 89
MSRTIELFNNLMSVVEKSEKGNFYFKDVITSMGTKARIFSHQIASYTDWLQDDALECRGIMFELNDKNEPVRIMA
RPMQKFFNLKENPMTIGLDLTKMIGLMEKADGSLISSYHDQGYVYLKSKTSIFSDQANKAMALLNSPAYEKLRDA
IVRAGSDFTFNMEYVGPSNRVVLPYEEEELIVLNVRHNETGQYVEFSTLLDDPLIRHRMIGVYPCPDWSKVTPEE
WEAATRAETDIEGVIGIMPDGQLFKLKTDWYSSLGRTKFSINNNKALHQSIKERASGALRGMFSDDNAALAKIEA
FESAYIDTVAKYHKICAEVFLRFARFLIVEVFAIEAQARMKDCRYLFSIVMQQYGRDWDGELAVEKIEEHIIKEYAT
YVPMAYR
SEQ ID NO: 90
MSRTIELFNNLMSVVEKSEKGNFYFKDVITSMGTKARIFSYFIASYTDWLQDDALECRGIMFELNDKNEPVRIMA
RPMQKFFNLKENPMTIGLDLTKMIGLMEKADGSLISSYHDQGYVYLKSKTSIFSDQANKAMALLNSPAYEKLRDA
IVRAGSDFTFNMEYVGPSNRVVLPYEEEELIVLNVRHNETGQYVEFSTLLDDPLIRHRMIGVYPCPDWSKVTPEE
WEAATRAETDIEGVIGIMPDGQLFKLKTDWYSSLGRTKYSINNNKALNQSIKERASVALRGMFSDDNAALAKIEA
FESAYIDTVAKYHKICAEVFLRFARFLIVEVFAIEAQARMKDCRYLFSIVMQQYGRDWDGELAVEKIEEHIIKEYAT
YVPMAYR
SEQ ID NO: 91
MSRTIELFNNLMSVVEKSEKGNFYFKDVITSMGTKARIFSHNIASYTDWLQDDALECRGIMFELNDKNEPVRIMA
RPMQKFFNLKENPMTIGLDLTKMIGLMEKADGSLISSYHDQGYVYLKSKTTIFSDQANKAMALLNSPAYEKLRDA
IVRAGSDFTFNMEYVGPSNRVVLPYEEEELIVLNVRHNETGQYVEFSTLLDDPLIRHRMIGVYPCPDWSKVTPEE
WEAATRAETDIEGVIGIMPDGQLFKLKTDWYSSLGRTKYSINNNKALHQSIKERASVDLRGMFSDDNAALAKIE
AFESAYIDTVAKYHKICAEVFLRFARFLIVEVFAIEAQARMKDCRYLFSIVMQQYGRDWDGELAVEKIEEHIIKEYA
TYVPMAYR
SEQ ID NO: 92
MSRTIELFNNLMSVVEKSEKGNFYFKDVITSMGTKARIFSHKIASYTDWLQDDALECRGIMFELNDKNEPVRIMA
RPMQKFFNLKENPMTIGLDLTKMIGLMEKADGSLISSYHDQGYVYLKSKAAIFSDQANKAMALLNSPAYEKLRDA
IVRAGSDFTFNMEYVGPSNRVVLPYEEEELIVLNVRHNETGQYVEFSTLLDDPLIRHRMIGVYPCPDWSKVTPEE
WEAATRAETDIEGVIGIMPDGQLFKLKTDWYSSLHRTKDSINNNKALFQSIKERASDDLRGMFSDDNAALAKIE
AFESAYIDTVAKYHKICAEVFLRFARFLIVEVFAIEAQARMKDCRYLFSIVMQQYGRDWDGELAVEKIEEHIIKEYA
TYVPMAYR
SEQ ID NO: 93
ACTCATCGATC
SEQ ID NO: 94
ACTCATCGATT
SEQ ID NO: 95
MEEDKAYWNKDAQDALDKQLGIKLREKQAKNVIFFLGDGMSLSTVTAARIYKGGLTGKFEREKISWEEFDFAAL
SKTYNTDKQVTDSAASATAYLTGVKTNQGVIGLDANTVRTNCSYQLDESLFTYSIAHWFQEAGRSTGVVTSTRV
THATPAGTYAHVADRDWENDSDVVHDREDPEICDDIAEQLVFREPGKNFKVIMGGGRRGFFPEEALDIEDGIPG
EREDGKHLITDWLDDKASQGATASYVWNRDDLLAVDIRNTDYLMGLFSYTHLDTVLTRDAEMDPTLPEMTKVA
IEMLTKDENGFFLLVEGGRIDHMHHANQIRQSLAETLDMEEAVSMALSMTDPEETIILVTADHGHTLTITGYADR
NTDILDFAGISDLDDRRYTILDYGSGPGYHITEDGKRYEPTEEDLKDINFRYASAAPKHSATHDGTDVGIWVNGP
FAHLFTGVYEENYIPHALAYAACVGTGRTFCD
SEQ ID NO: 96
ACTCATCGATA
SEQ ID NO: 97
ACTCATCGATAA
SEQ ID NO: 98
ACTCATCGATAA
SEQ ID NO: 99
ACTIAGACCACG
SEQ ID NO: 100
MLQMNLEELRRIQEEMSRSVVLEDLIPLEELEYVVGVDQAFISDEVVSCAVKLTFPELEVVDKAVRVEKVTFPYIPT
FLMFREGEPAVNAVKGLVDDRAAIMVDGSGIAHPRRCGLATYIALKLRKPTVGITKKRLFGEMVEVEDGLWRLLD
GSETIGYALKSCRRCKPIFISPGSYISPDSALELTRKCLKGYKLPEPIRIADKLTKEVKRELTPTSKLK
SEQ ID NO: 101
GACCACG

1. A process for producing a single-stranded oligonucleotide product having at least one modified nucleotide residue, comprising:
a) providing a pool of oligonucleotides (I) that comprises segments of the product sequence, wherein at least one segment of the product sequence contains at least one modified nucleotide residue, and wherein each segment has been produced by enzymatic synthesis or solid phase synthesis;
b) providing a template oligonucleotide (II) complimentary to the sequence of the single-stranded oligonucleotide product, said template having a property that allows it to be separated from the product and recycled for use in future reactions;
c) contacting (I) and (II) in conditions to allow annealing of the segment oligonucleotides to the template oligonucleotide;
d) joining the segment oligonucleotides by using a ligase to form the product;
e) changing the conditions to denature the annealed template and any impurity oligonucleotide strands, and separating the impurities;
f) changing the conditions to denature the annealed template and product oligonucleotide strands, and separating the single-stranded oligonucleotide product; and
g) recycling the template oligonucleotide for use in future reactions.

2. A process according to claim 1, wherein one or more or all segment oligonucleotides is produced by enzymatic synthesis.

3. A process according to claim 1 or 2, wherein one or more segment oligonucleotides is produced by using a single-stranded ligase (ssLigase), optionally an RNA ligase.

4. A process according to claim 3, wherein the process for producing a segment oligonucleotide using a ssLigase comprises:
(i) adding a nucleotide, a dinucleotide, a trinucleotide or a tetranucleotide; which has either a phosphate, thiophosphate or dithiophosphate moiety at the 3′ end; and either a phosphate, thiophosphate or dithiophosphate moiety at the 5′ end; to the 3′-OH of a single-stranded oligonucleotide primer by using a ssLigase;
(ii) removing the 3′-phosphate, 3′-thiophosphate or dithiophosphate by using a phosphatase;
(iii) repeating steps (i) and (ii) to extend the oligonucleotide to produce the segment sequence; and
(iv) releasing the segment oligonucleotide from the oligonucleotide primer by using a sequence specific endonuclease.

5. A process according to claim 4, wherein a 3′,5′ nucleotide bisphosphate, having one or more of either of the phosphate oxygens substituted by sulphur, is added to the 3′-OH of a single-stranded oligonucleotide primer by using a ssLigase in step (i).

6. A process according to claim 4 or 5, wherein a 3′,5′ bisthiophosphate, a 3′-phosphate-5′-thiophosphate, a 3′-thiophosphate-5′-phosphate, a 3′,5′ bisdithiophosphate, a 3′-phosphate-5′-dithiophosphate or a 3′-dithiophosphate-5′-phosphate is added to the 3′-OH of a single-stranded oligonucleotide primer by using a ssLigase in step (i).

7. A process according to any preceding claim, wherein one or more or all segment oligonucleotides is produced using a transferase.

8. A process according to claim 7, wherein the process for producing a segment oligonucleotide using a transferase comprises:
(i) adding a nucleotide-5′-triphosphate, alpha-thiotriphosphate or alphadithiotriphosphate, which has a protecting group on its 3′-OH, to the 3′-OH of a single-stranded oligonucleotide primer, by using a transferase;
(ii) deprotecting the 3′-position to regenerate the 3′-OH;
(iii) repeating steps (i) and (ii) to extend the oligonucleotide to produce the segment sequence;
(iv) releasing the segment oligonucleotide from the oligonucleotide primer by using a sequence specific endonuclease.

9. A process according to any preceding claim, wherein each segment is produced by enzymatic synthesis, optionally wherein each segment is produced by using a ssLigase.

10. A process according to any preceding claim, wherein the process is semi-continuous or continuous.

11. A process according to any preceding claim, wherein the product is produced at gram or kilogram scale and/or the process is carried out in a reactor or at least 1 L.

12. A process according to any preceding claim, whereby the denaturing results from a temperature increase, a change in the pH, or a change in the salt concentration in a buffering solution.

13. A process according to claim 12, wherein the process includes two steps of increasing the temperature: i) to denature template:impurity duplexes and ii) to denature template:product duplexes.

14. A process according to any preceding claim, wherein the segments are 3 to 15 nucleotides long.

15. A process according to any preceding claim, wherein the length of the product is 10 to 200 nucleotides long, optionally 20 to 30 nucleotides long, optionally 20 to 25 nucleotides long.

16. A process according to claim 155, wherein said product is 20 nucleotides long and comprises three segment oligonucleotides comprising:
(i) a 5′ segment that is 7 nucleotides long, a central segment that is 6 nucleotides long and a 3′ segment that is 7 nucleotides long;
(ii) a 5′ segment that is 6 nucleotides long, a central segment that is 8 nucleotides long and a 3′ segment that is 6 nucleotides long;
(iii) a 5′ segment that is 5 nucleotides long, a central segment that is 10 nucleotides long and a 3′ segment that is 5 nucleotides long;
(iv) a 5′ segment that is 4 nucleotides long, a central segment that is 12 nucleotides long and a 3′ segment that is 4 nucleotides long; or
(v) a 5′ segment that is 3 nucleotides long, a central segment that is 14 nucleotides long and a 3′ segment that is 3 nucleotides long.

17. A process according to any preceding claim, wherein the template is attached to a support material.

18. A process according to claim 17, wherein the support material is a soluble support material, optionally wherein the support material is selected from the group consisting of polyethylene glycol, a soluble organic polymer, DNA, a protein, a dendrimer, a polysaccharide, an oligosaccharide, and a carbohydrate.

19. A process according to claim 17, wherein the support material is an insoluble support material, optionally wherein the insoluble support material is selected from the group consisting of: a glass bead, a polymeric bead, a fibrous support, a membrane, a streptavidin coated bead, and cellulose, or is part of the reaction vessel itself, e.g. a reaction wall.

20. A process according to any one of claims 17 to 19, wherein multiple, repeated copies of the template are attached in a continuous manner via a single attachment point to the support material.

21. A process according to any one of claims 17 to 19, wherein the template is attached to the support material at multiple attachment points.

22. A process according to any preceding claim, wherein the modification is at the 2′ position of the sugar moiety, optionally selected from the group consisting of 2′-F, 2′-OMe, 2′-MOE, and 2′-amino, or wherein the oligonucleotide comprises a PMO, a LNA, a PNA, a BNA, or a SPIEGELMER.

23. A process according to any preceding claim, wherein the modification is in the nucleobase, optionally wherein the modification is selected from the group consisting of a 5-methyl pyrimidine, a 7-deazaguanosine and an abasic nucleotide.

24. A process according to any preceding claim, wherein the modification is in the backbone, optionally wherein the modification is selected from the group consisting of phosphorothioate, phosphoramidate and phosphorodiamidate.

25. A process according to any preceding claim, wherein the resulting single-stranded oligonucleotide product is at least 90% pure, optionally wherein the single-stranded oligonucleotide product is at least 95% pure, optionally wherein the single-stranded oligonucleotide product is at least 98% pure.

26. A process according to any preceding claim, wherein the process produces a gapmer.

27. A process for producing a double-stranded oligonucleotide product, wherein 2 complimentary single-stranded oligonucleotides produced by the process of any preceding claim are mixed under conditions to allow annealing.

28. A process as claimed in any preceding claim, wherein the process produces a therapeutic oligonucleotide.
